(19) United States
(12) Patent Application Publication
(10) Pub. No.: US 2022/0154219 A1
(43) Pub. Date: May 19, 2022

(54) GENE DELIVERY PARTICLES TO INDUCE TUMOR-DERIVED ANTIGEN PRESENTING CELLS

(71) Applicant: The Johns Hopkins University, Baltimore, MD (US)

(72) Inventors: Jordan J. Green, Baltimore, MD (US); Stephany Yi Tzeng, Baltimore, MD (US); David Wilson, Baltimore, MD (US); Randall A. Meyer, Baltimore, MD (US)

(21) Appl. No.: 17/441,188

(22) PCT Filed: Mar. 23, 2020
(86) PCT No.: PCT/US2020/024220

§ 371 Date: Sep. 20, 2021

Related U.S. Application Data
(60) Provisional application No. 62/822,385, filed on Mar. 22, 2019.

(51) Int. Cl. C12N 15/88; A61K 39/39

(52) U.S. Cl. CPC C12N 15/88; A61K 2039/5152; A61K 39/39

(57) ABSTRACT

Synthetic, biodegradable nanoparticles (NPs) encapsulating at least one of a signal 1 protein, a signal 2 protein, and/or a signal 3 protein are disclosed, which, when transfected into one or more a cancer cells, reprogram the one or more cancer cells into “tumor-derived APCs” in vivo to activate T-cells and natural killer (NK) cells for systemic tumor rejection. The NPs can be used for treating cancers, in particular metastatic cancers.

### FEDERALLY SPONSORED RESEARCH OR DEVELOPMENT

This invention was made with government support under grant number EB022148 awarded by the National Institutes of Health. The government has certain rights in the invention.

### BACKGROUND

Immunotherapy has shown clinical success in treating cancer that is unresponsive to conventional treatment. Redman et al., 2016; Nanda et al., 2016. This approach requires immune cells to be activated to recognize tumor antigens, after which they can seek out and destroy cancer cells. Mellman et al., 2011; Smyth et al., 2001. For T-cell immunotherapy, antigen-presenting cells (APCs) normally activate CD8⁺ cytotoxic T-cells by presenting a signal 1, consisting of a major histocompatibility complex (MHC) I molecule with an antigen peptide, a co-stimulatory signal 2 that directs the action of the T-cells upon recognition of the tumor antigen, see Ben-Akiva et al., 2017, and a secreted signal 3 for recruitment and differentiation of immune cells (see, for example, FIG. 1). Curtsinger et al., 1999. Natural killer (NK) cells, by contrast, are activated in an antigen-independent manner, depending on the balance of activating and inhibiting signals they receive in the form of signal 2 and signal 3.

Attempts to engineer live APCs for enhanced T-cell stimulation, see Banchereau and Palucka, 2005; Kantoff et al., 2010; and Anguille et al., 2014, are hampered by high costs and risks of ex vivo manipulation of primary immune cells. In vivo APC manipulation is limited by the technical difficulties of targeting these cells. See Anguille et al., 2014; Tacken et al, 2007. Another method, the fabrication of artificial APCs (aAPCs), see Wang et al., 2017; Eggermont et al., 2014, must tackle the complexities of using synthetic particles to mimic cells. The only FDA-approved aAPC therapy still requires costly ex vivo manipulation of patient cells. Kantoff et al., 2010. Furthermore, current aAPC approaches require knowledge of the correct tumor antigen(s) needed to stimulate an immune response against a patient's tumor a priori during particle fabrication, yet this patient-specific knowledge is unavailable and/or difficult to obtain. Accordingly, at present, T-cell immunotherapy is not amenable to treating patients with a broad range of cancers and histocompatibilities.

### SUMMARY

In some aspects, the presently disclosed subject matter provides a composition comprising at least one of a first genetic element that encodes a signal 2 protein and a second genetic element that encodes a signal 3 protein encapsulated in a nanoparticle comprising a cationic biomaterial or biomaterial blend. In certain aspects, the composition further comprises a third genetic element that encodes a signal 1 protein.

In particular aspects, the signal 2 protein is a cell surface bound protein that regulates immune cells. In more particular aspects, the signal 2 protein is selected from the group consisting of 4-1BBL, CD80, CD86, and OX40L.

In other aspects, the signal 3 protein is a secreted protein that regulates immune cells. In particular aspects, the signal 3 protein comprises a cytokine. In more particular aspects, the cytokine comprises an interleukin. In yet more particular aspects, the signal 3 protein is selected from the group consisting of IL-2, IL-12, IL-6, IL-7, IL-15, IL-18, IL-21, IFN-α, and IFN-β.

In certain aspects, the signal 1 protein is major histocompatibility complex (WIC) I or WIC II.

In some aspects, the cationic biomaterial comprises one or more cationic polymers. In certain aspects, the one or more cationic polymers comprises one or more cationic biodegradable polymers. In more certain aspects, the one or more cationic degradable polymers comprises one or more poly(beta-amino ester)s (PBAEs).

In particular aspects, the one or more PBAEs comprises a compound of formula (I)

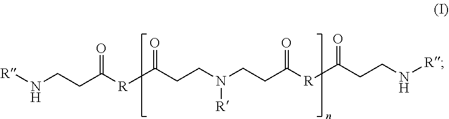

wherein: n is an integer from 1 to 10,000; each R is independently selected from the

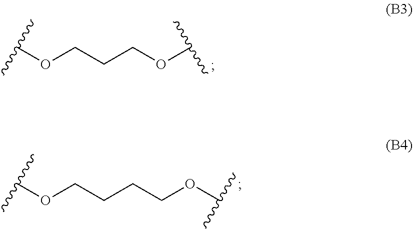 [SMILES: *OCCCO* ; *OCCCCO*]

group consisting of:

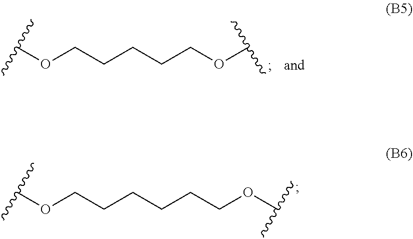 [SMILES: *OCCCCCO* ; *OCCCCCCO*]

each R′ is independently selected from the group consisting of:

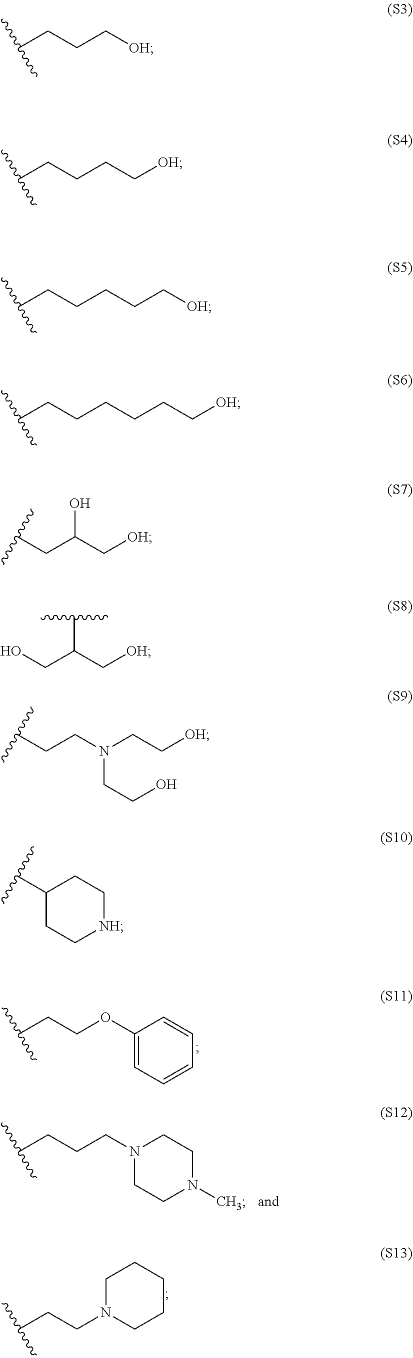

each R″ is independently selected from the group consisting of:

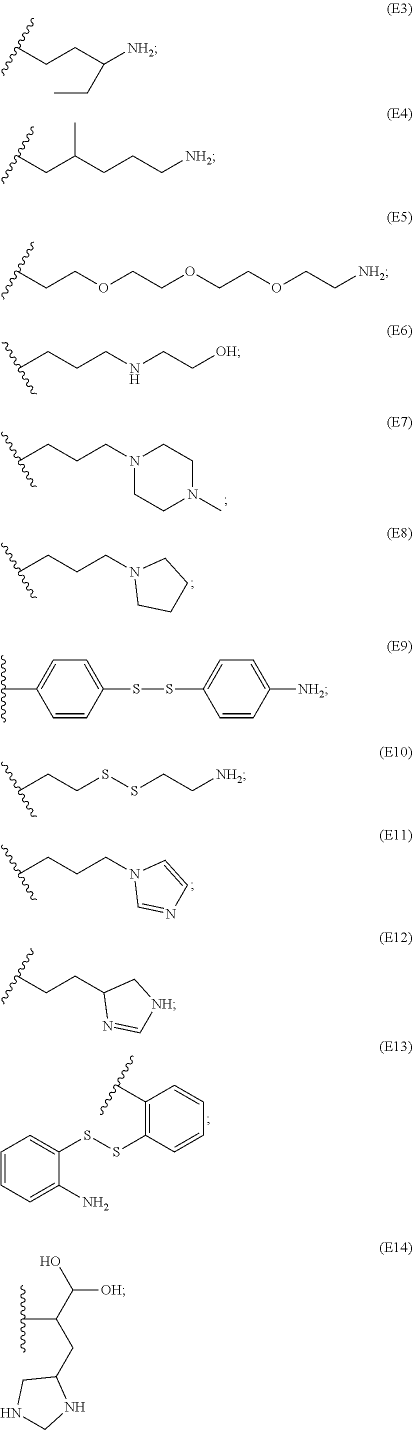

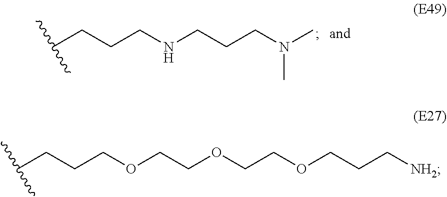 [SMILES: *CCCNCCCN(C)C ; *CCCOCCOCCOCCCN]

and pharmaceutically acceptable salts thereof.

In more particular aspects, the one or more PBAEs is selected from the group consisting of:

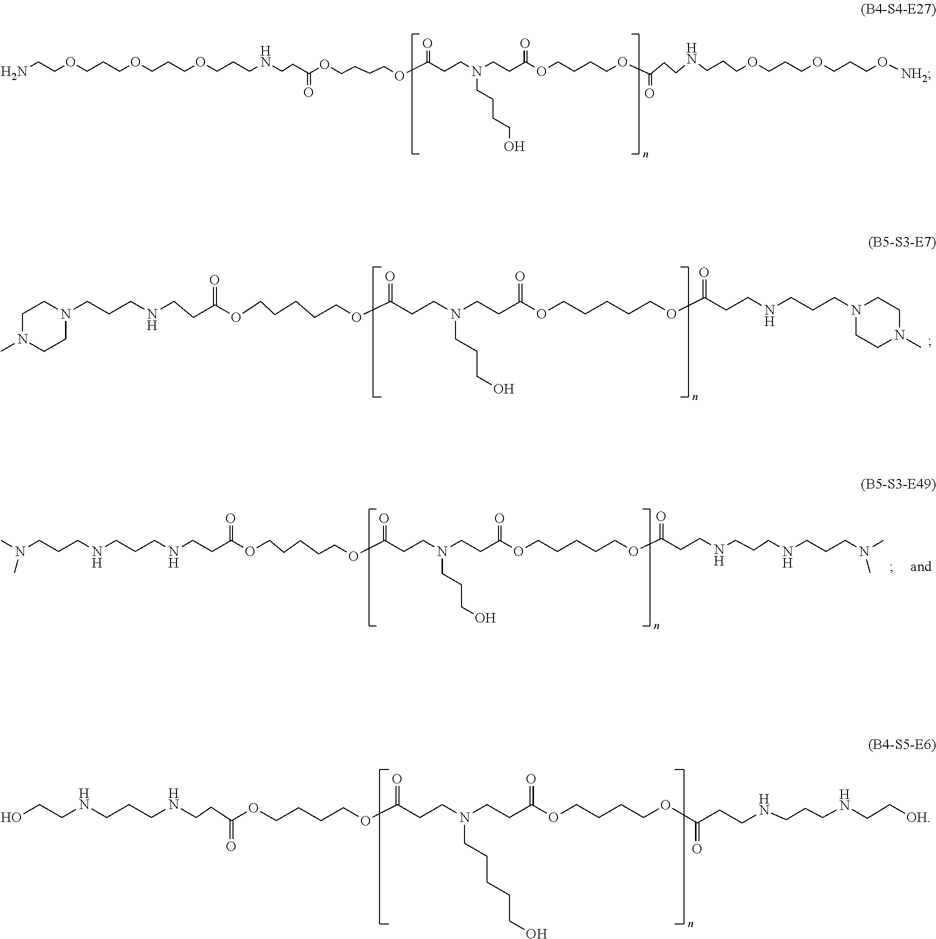

In certain aspects of the compound of formula (I), n is selected from the group consisting of an integer from 1 to 1,000; an integer from 1 to 100; an integer from 1 to 30; an integer from 5 to 20; an integer from 10 to 15; and an integer from 1 to 10. In other aspects, the nanoparticle has a size ranging from about 20 nm to about 50 nm; from about 50 nm to about 200 nm; or from about 200 to about 500 nm.

In other aspects, the presently disclosed subject matter provides a method for reprogramming one or more cancer cells into one or more tumor-derived antigen-presenting cells (tAPCs), wherein the one or more tAPCs mimic a natural antigen-presenting cell (APC) and direct an immune response against themselves and other cancer cells, the method comprising transfecting the one or more cancer cells with the presently disclosed nanoparticle composition.

In certain aspects, the transfection of the one or more cancer cells promotes an immune cell activation against one or more antigens expressed on the one or more cancer cells. In particular aspects, the one or more tAPCs activate an antigen-specific T-cell response against WIC I+ tumor cells. In other aspects, the one or more tAPCs provide an activating signal to one or more natural killer (NK) cells to induce anti-tumor cytotoxicity therein. In particular aspects, the one or more tAPCs activate an antigen-independent NK cell response against MHC I−/low tumor cells. In certain aspects, the presently disclosed method further induces a systemic immune response resulting in cell death of distant metastases.

In other aspects, the presently disclosed subject matter provides a method for treating cancer, the method comprising administering to a subject in need of treatment thereof the presently disclosed nanoparticle composition.

In yet other aspects, the presently disclosed subject matter provides a pharmaceutical formulation of the presently disclosed nanoparticle composition in a pharmaceutically acceptable carrier.

In other aspects, the presently disclosed subject comprises a kit comprising the presently disclosed nanoparticle composition.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

The patent or application file contains at least one drawing executed in color. Copies of this patent or patent application publication with color drawings will be provided by the Office upon request and payment of the necessary fee.

Having thus described the presently disclosed subject matter in general terms, reference will now be made to the accompanying Figures, which are not necessarily drawn to scale, and wherein:

FIG. 1 is a scheme depicting a representative presently disclosed method for transfecting cancer cells with a signal 2 co-stimulatory molecule and a signal 3 immunostimulatory cytokine, reprogramming them into “tumor-derived APCs” (tAPCs) that mimic classical APCs and direct an immune response against themselves and other cancer cells. Even in the absence of MHC expression (signal 1) by tumors, the presently disclosed method can provide activating signals to NK cells to induce anti-tumor cytotoxicity;

FIG. 2 shows representative poly(beta-amino esters) (PBAEs) suitable for use with the presently disclosed formulations and methods. The presently disclosed PBAEs comprise at least one backbone monomer (designated herein as “B”), at least one side-chain monomer (designated herein as “S”), and at least one end-cap monomer (designated herein as “E”). These three classes of monomers can be synthesized into a library of different PBAEs with different combinations of “B,” “S,” and “E” via combinatorial chemistry;

FIG. 3A, FIG. 3B, FIG. 3C, FIG. 3D, and FIG. 3E are murine malignant melanoma cells (FIG. 3A, FIG. 3B, and FIG. 3C) and murine malignant glioma cells (FIG. 3D and FIG. 3E), which, when co-transfected with signal 2 and signal 3, robustly express both transgenes. The amount of expression of each transgene can be tailored by changing the ratio of plasmids co-delivered to the cells;

FIG. 4 shows B16-F10 melanoma cells that were transfected with either a control plasmid (red fluorescent protein), signal 2 (4-1BBL), or one of two different signal 3 cytokines (IL-2 or IL-12). The transfected B16-F10 melanoma cells were then co-cultured with either primary NK or CD8⁺ T cells isolated from wild-type C57BL/6 mice. Interferon-γ (IFN-γ) secreted by activated lymphocytes was detected in the media after co-culture with B16-F10 cells transfected with both signal 2 and signal 3. Importantly, signal 2 on its own was not sufficient to cause significant activation. Further, the combination of both signal 2 and signal 3 appears to be synergistic;

FIG. 5 demonstrates that the presently disclosed “tumor-derived APC” (tAPC) reprogramming strategy is effective in vitro in multiple different cancer models, including melanoma (B16-F10), glioma (GL261), and triple-negative breast cancer (4T1). Cancer cells were transfected with signal 2 (e.g., 4-1BBL), signal 3 (e.g., IL-12), or a combination of both. The transfected cells were then co-cultured with either primary NK or CD8⁺ T cells isolated from the spleens of wild-type syngeneic mice (C57BL/6 for GL261 and B16-F10 or Balb/c for 4T1). Interferon-γ (IFN-γ) secreted by activated lymphocytes was detected in the media after co-culture with tAPCs, demonstrating that the presently disclosed strategy may be viable for multiple different tumor types;

FIG. 6 shows C57BL/6 mice that were inoculated with B16-F10 malignant melanoma tumors. Intratumoral injection of NPs encoding signal 2 (4-1BBL), signal 3 (IL-12), or both, along with intraperitoneal injection of anti-PD-1 antibody, resulted in slowed tumor growth compared to injection of control NPs. While signal 2 alone did not cause statistically significantly slower tumor growth compared to the control, it did lead to one long-term survivor by 60 days. Signal 3 alone and particularly in combination with signal 2 leads to significantly slowed tumor growth and longer survival, with two long-term survivals in the signal 2+3 group at 60 days; (D) Some mice treated with signals 2 and 3 completely cleared their tumor and displayed vitiligo-like patterns of white fur starting at the injection site and spreading over time to more distant patches of fur at 33 days (left) and 77 days (right);

FIG. 7 shows Balb/c mice that were inoculated with 4T1 breast cancer tumors. Intratumoral injection of NPs encoding signal 2 (4-1BBL), signal 3 (IL-12), or both, along with intraperitoneal injection of anti-PD-1 antibody, does not result in tumor clearance at early time points, indicating that a different combination of signal 2 and signal 3 and/or additional combinations may be required for efficacy in the 4T1 model. Notably, while the 4T1 in vitro co-culture studies (FIG. 5) yielded detectable results indicating T- and NK-cell activation with 4-1BBL and IL-12, the results were weaker in this cell line than in the B16-F10 model. This observation suggests that further optimization of the delivered immune-stimulatory signals may be required depending on the tumor type and that specific biomaterials types and nanoparticle formulation conditions are needed to have in vivo efficacy among different cancer types;

FIG. 8A shows B16-F10 cells transfected by presently disclosed PBAEs;

FIG. 8B demonstrates that surface 4-1BBL and secreted IL-2 can be co-expressed from co-transfected cells at varying ratios;

FIG. 8C shows that co-transfected cells cause higher IFN-γ expression in co-cultured CD8+ T-cells in vitro;

FIG. 8D shows that intratumoral injection of only signal 2 DNA (e.g., 4-1BBL) in an in vivo flank B16-F10 tumor resulted in slowed tumor growth compared to the control;

FIG. 9 shows the in vitro transfection of B16-F10 melanoma cells including % cells transfected (left panel); normalized mean fluorescence units (MFI) (center panel); and viability (right panel) at 30 w/w, 60 w/w, and 90 w/w of PBAE nanoparticle designated as B-S-E;

FIG. 10A, FIG. 10B, FIG. 10C, and FIG. 10D demonstrate that: (FIG. 10A) PBAEs deliver GFP to cancer cells in vitro to mouse melanoma, triple-negative breast cancer, and colorectal carcinoma; (FIG. 10B) NPs show high delivery specificity for human cancer cells over healthy human cells, a result that is consistent across 4 primary brain cancer lines and 3 healthy human brain samples and is independent of differences in cell division rate; (FIG. 10C) When liver cancer cells (red) are co-cultured with non-cancerous hepatocytes (no color) and then co-transfected, PBAE NPs successfully deliver the gene (green) with high specificity to cancer cells; and (FIG. 10D) When injected into tumors, PBAE NPs transfect cancer cells but not surrounding healthy tissue;

FIG. 11 Fluorescence micrograph confirms the high in vitro transfection of B16-F10 melanoma cells by 4-4-27, 60 w/w particles);

FIG. 12 shows the in vivo transfection of B16-F10 melanoma tumors with intratumoral injections of 4-4-27 PBAE nanoparticles;

FIG. 13 presents a protocol for co-culturing B16-F10 with NK- or T-cells;

FIG. 14 shows the activation of NK cells and CD8+ T-cells via co-culture with transfected B16-F10. These results demonstrate very low activation of NK cells from 4-1BBL; low activation of NK and CD8+ T-cells from IL-2; and significantly (p<0.001) higher activation of both NK and CD8+ T-cells from co-expression of 4-1BBL and IL-2;

FIG. 15A and FIG. 15B show: B16-F10 tumor-bearing mice treated with 4-4-27 PBAE NPs encoding of 4-1BBL and IL-2 plus systemic anti-PD-1 antibody show synergy and durable systemic immunity. Long-term survivors were protected against a second tumor challenge on the opposite flank compared to age-matched controls. FIG. 15C: CD8⁺ T cells were isolated from the spleen of a normal mouse, a mouse with an untreated tumor, or mouse with a tumor treated with reprogramming NPs, and these T cells were co-cultured with B16-F10 cells in vitro. T cells from NP-treated mice were more strongly stimulated by B16-F10 cells in vitro;

FIG. 16A, FIG. 16B, FIG. 16C, FIG. 16D, FIG. 16E, and FIG. 16F demonstrate that PBAE nanoparticles transfect B16-F10 melanoma cells with reporter genes in vitro and in vivo. (FIG. 16A) Monomers used to synthesize PBAEs are shown. (FIG. 16B) PBAE nanoparticles were used to transfect B16-F10 cells with GFP DNA using varying mass ratios (w/w) of PBAE to DNA. Mean±SE are shown (n=4). (FIG. 16C) Fluorescence micrographs were taken of the leading formulations. (Scale bars: 200 μm) (FIG. 16D and FIG. 16E) subcutaneous tumors in C57BL/6 mice were transfected with fLuc using leading nanoparticles and imaged by IVIS after 24 h. Mean±SE are shown (n=4 for control; n=6 for all other groups). (FIG. 16F) TEM was used to visualize the nanoparticles. (Scale bar: 100 nm.) DLS and NTA were used to measure size, and electrophoretic mobility was used to measure zeta potential (ZP). Mean±SE are shown (n=3);

FIG. 17A, FIG. 17B, FIG. 17C, FIG. 17D, FIG. 17E, and FIG. 17F demonstrate that B16-F10 melanoma cells transfected to express signals 2 and 3 in vitro cause activation of primary T and NK cells. (FIG. 17A, FIG. 17B, and FIG. 17C) B16-F10 cells were transfected with 5-3-49 PBAE/DNA nanoparticles encoding IL-12, 4-1BBL, or both. Secreted IL-12 was measured by ELISA, and surface-bound 4-1BBL was measured by flow cytometry. (FIG. 17D) Transfection with a mixture of the 4-1BBL and IL-12 plasmids results in a synergistic effect greater than the additive effects of each plasmid on its own. (FIG. 17E and FIG. 17F) Across different doses of total plasmid per well, the effect (IFN-γ secretion) of 4-1BBL and IL-12 plasmids in combination is consistently higher than the added effects of 4-1BBL transfection alone and IL-12 transfection alone. For FIG. 17A, FIG. 17B, FIG. 17C, and FIG. 17D, statistically significant differences were measured by one-way ANOVA with Dunnett post-tests comparing to the control (100% Ctrl). All bar graphs show mean±SE. Four (n=4) replicates were used per group. For FIG. 17F, a two-way ANOVA was performed, with Dunnett post-tests comparing to the control (1:1 Additive Effects). Asterisk colors correspond to the group found to be significantly different from the control at that dose. *P<0.05; **P<0.01; ***P<0.001; ****P<0.0001;

FIG. 18A, FIG. 18B, and FIG. 18C demonstrate that in vivo tAPC reprogramming significantly inhibits tumor growth and leads to long-term survival. (FIG. 18A) IFN-γ was detectable in the tumor interstitial fluid 14 d after tumor inoculation in treated groups (n=4). (FIG. 18B) Of mice treated with anti-PD-1, slower tumor growth was measured in groups treated with IL-12 nanoparticles (significance marked by #) or 4-1BBL/IL-12 nanoparticles (significance marked by *). *P<0.05; ** or ##: P<0.01; **** or ####: P<0.0001. Significance was calculated by two-way repeated measures ANOVA with a Dunnett post-test to compare against animals treated with control nanoparticles and anti-PD-1. (FIG. 18C) Mice treated with IL-12 or 4-1BBL/IL-12 nanoparticles and anti-PD-1 survived significantly longer than the control (P=0.0018). All error bars are SEM;

FIG. 19A, FIG. 19B, FIG. 19C, FIG. 19D, FIG. 19E, FIG. 19F, and FIG. 19G demonstrate that local immune cell populations measured by qPCR indicate an antitumor cytotoxic response caused by tAPC reprogramming NPs. (FIG. 19A) Genes indicating the presence of total tumor-infiltrating leukocytes, total T cells, and CD8+ T cells increased between 14 and 18 d in groups that received reprogramming nanoparticles. (FIG. 19B) Treatment with signal 2 and/or 3 nanoparticles results in elevated expression of genes indicating increased proportions of infiltrating immune cells in the tumor. (FIG. 19C) Normalizing CD3c expression to CD45 expression suggests that a greater proportion of TILs are T cells in animals treated with tAPC reprogramming nanoparticles. (FIG. 19D) The ratio of CD8a to CD4 expression suggests a more cytotoxic immune response was after treatment with tAPC-reprogramming nanoparticles. (FIG. 19E) The high ratio of IFN-γ to TGF-β expression in tAPC-treated animals suggests a bias toward Th1 antitumor activation, and (FIG. 19F and FIG. 19G) the lower ratio of Foxp3 to CD3ε and CD4 expression in those groups also suggests a decrease in Tregs at the tumor site. For all, mean±SE of four (n=4) replicates is shown. *P<0.05; **P<0.01; ****P<0.0001; statistically significant differences were measured by one-way ANOVA with Dunnett post-tests comparing to the control (Ctrl NPs);

FIG. 20A, FIG. 20B, FIG. 20C, FIG. 20D, FIG. 20E, FIG. 20F, FIG. 20G, and FIG. 20H demonstrate that flow cytometry after 14 d confirms a cytotoxic immune response in the tumor microenvironment due to tAPC reprogramming NPs. (FIG. 20A and FIG. 20B) Mice treated with reprogramming nanoparticles, particularly in combination with anti-PD-1, had more TILs and, within TILs, more T cells. (FIG. 20C and FIG. 20D) tAPC reprogramming resulted in a more CD8+ cytotoxic T cells after 14 d. (FIG. 20E and FIG. 20F) Among CD3− TILs, the NK cell population was greater in tAPC-reprogrammed tumors. (FIG. 20G and FIG. 20H) The CD4+ population was significantly greater among T cells in tAPC-treated tumors, but the Foxp3+ population was not increased in tumors injected with signal 3 or signal 2/3 nanoparticles. Signal 2 nanoparticles in combination with anti-PD-1 did increase the Foxp3+ population. *P<0.05; **P<0.01; ***P<0.001; statistically significant differences were measured by one-way ANOVA with Dunnett post-tests comparing to the control (Ctrl NPs). For all bar graphs, mean±SE of four (n=4) replicates is shown;

FIG. 21A, FIG. 21B, FIG. 21C, FIG. 21D, FIG. 21E, and FIG. 21F demonstrate that local tAPC reprogramming leads to a durable and systemic antitumor immune response. (FIG. 21A) Survivors rechallenged with new s.c. B16-F10 tumors on the opposite flank resisted tumor formation compared to untreated control mice and (FIG. 21B) survived significantly longer after rechallenge. **P<0.01; ***P<0.001; ****P<0.0001. (FIG. 21C) One of the long-term survivors developed a vitiligo-like patch of depigmented fur at the site of the eliminated tumor, which began to spread to other patches of fur, indicating a cytotoxic immune response at more distant locations. Statistically significant differences in the growth rate were measured by two-way repeated-measures t-tests with Holm-Sidak tests to correct for multiple comparisons. Differences in survival were calculated by the Mantel-Cox log-rank test. (FIG. 21D) CD8+ T cells isolated from tAPC-treated tumor-bearing mice were activated more effectively after in vitro stimulation with B16-F10 cells than CD8+ T cells from tumor-bearing mice administered control nanoparticles or checkpoint inhibition alone. (FIG. 21E and FIG. 21F) The splenic CD8+ T cell population was more specific for gp100, a common melanoma antigen. PE, phycoerythrin. The graphs show mean±SE. Significance was calculated by one-way ANOVA with Dunnett post-tests against the “Ctrl NPs” group. *P<0.05;

FIG. 22A, FIG. 22B, FIG. 22C, and FIG. 22D. The tAPC strategy is effective in multiple tumor models. (FIG. 22A) MC38 cells could be transfected in vitro and in vivo after i.t. injection with nanoparticles. (FIG. 22B) Transfected MC38 cells show the same trends in activating splenocytes as B16-F10 cells, with a synergistic effect seen between signals 2 and 3. For each time point (red and blue), one-way ANOVA was done with Dunnett post-tests comparing to “Ctrl.” ***P<0.001, ****P<0.0001. (FIG. 22C) MC38 tumors grew more slowly after tAPC nanoparticle treatment. Statistically significant differences in the growth rate were measured by two-way repeated-measures t-tests with Holm-Sidak tests to correct for multiple comparisons. *P<0.05. (FIG. 22D) 41BBL/IL12 NPs+αPD1-treated mice survived significantly longer than control NPs+αPD1-treated mice and also are able to 100% reject a rechallenge of the tumor on the opposite flank. Differences in survival were calculated by the Mantel-Cox log-rank test with Bonferroni correction for multiple comparisons;

FIG. 23 shows that after an initial screen (FIG. 16), the PBAEs considered for further study were those that caused <20% toxicity (>80% viability) in B16-F10 cells. The formulations that met this criterion were then ranked by percent of cells transfected and geometric mean GFP fluorescence intensity. Four (n=4) replicates were tested per group. Mean±standard error are shown;

FIG. 24 shows tumor growth in B16-F10 melanoma-bearing mice treated with either control (fLuc) nanoparticles alone or control nanoparticles along with anti-PD-1 checkpoint blockade. Anti-PD-1 slows tumor growth slightly, but differences are not statistically significant. Significance was measured by a two-way repeated measures t-test with Sidak-Bonferroni correction for multiple comparisons. Results were considered significant when p<0.05. Mean±standard error is shown for each point, and n=7 mice were measured per group;

FIG. 25 shows that qPCR performed 14 days after tumor inoculation exhibits strong trends in the expression of various immune cell markers. In general, tAPC-reprogrammed tumors showed higher expression of leukocyte (CD45) and lymphocyte (CD3c) markers, and there was an increase in the mRNA of genes expressed by cytotoxic lymphocytes, such as CD8+ T cells and NK cells (CD49b, CD94). The slight increase in CD4 expression indicates that CD4+ T cells, important for T-cell help, as well as immune regulation, also are stimulated by the tAPC reprogramming strategy, either via direct signaling by transfected cells or via downstream signaling by cells in a tumor microenvironment altered by NP-based gene delivery. All groups measure n=4 animals. Graphs show mean±standard error. *p<0.05; **p<0.01; ***p<0.001; statistically significant differences were measured by one-way ANOVA with Dunnett post-tests comparing to the control (Ctrl NPs);

FIG. 26 shows that B16-F10 subcutaneous (s.c.) tumors were established in mice and treated as described in Methods, then excised and analyzed for mRNA expression of markers indicative of the presence and/or activation state of innate immune cells as well as lymphocytes. Some monocyte, DC, macrophage, and neutrophil markers are elevated in the 4-1BBL-only group, but less so than many of the lymphocyte markers tested. The general trend seen for lymphocytes was also observable for many of the innate immunity and general infiltrating leukocyte markers, with slight upregulation in tumors treated with 4-1BBL, greater upregulation in tumors treated with IL-12, and the greatest effect in tumors treated with both. The difference among groups, however, was most striking for lymphocyte markers, particularly CD3 (T cells) and CD8 (CD8+ T cells) and IFN-γ as an activation marker. While this difference is not definitive proof of the mechanism of action proposed herein, it does suggest that one of, if not the-major effect of the presently disclosed treatment is on the recruitment, activation, and/or expansion of cytotoxic T lymphocytes, as the presently disclosed technology was originally designed to do;

FIG. 27A, FIG. 27B, FIG. 27C, FIG. 27D, FIG. 27E, and FIG. 27F demonstrate that flow cytometry on excised tumors 14 days after inoculation showed trends in immune cell populations corresponding to a cytotoxic or Th1 response in tAPC-treated groups. (FIG. 27A) Gating strategy used for all populations gated on all live cells. Separate samples were used for various stains to simplify the gating strategy. (FIG. 27B) Proportion of cells found in the tumor or in TILs. All groups measure n=4 animals. Graphs show mean±standard error. *p<0.05; **p<0.01; statistically significant differences were measured by one-way ANOVA with Dunnett post-tests comparing to the control (Ctrl NPs). (FIG. 27C-FIG. 27F) Flow cytometry pseudo-color plots showing differences among samples. Each plot shows the concatenation of all replicates to show the most representative image;

FIG. 28A, FIG. 28B, and FIG. 28C demonstrate that: (FIG. 28A) After 18 days, mice treated with reprogramming nanoparticles had more TILs and greater population of cytotoxic lymphocytes at the tumors site than the controls, measured by flow cytometry. (FIG. 28B) The Foxp3+ Treg population remained highest in the 4-1BBL nanoparticle-treated group after 18 days. (FIG. 28C) Dramatic differences in tumor size were seen 18 days after inoculation, with those in the 4-1BBL/IL-12 nanoparticles and anti-PD-1 group being significant smaller than controls. *p<0.05; **p<0.01; ***p<0.001; statistically significant differences were measured by one-way ANOVA with Dunnett post-tests comparing to the control (Ctrl NPs). For all bar graphs, mean±standard error of four (n=4) replicates is shown;

FIG. 29A, FIG. 29B, and FIG. 29C show that: (FIG. 29A-FIG. 29B) CD31 expression and LYVE-1 expression show the presence of blood vessels and lymphatic vessels throughout the tumor. (FIG. 29C) CD8 expression shows the presence of cytotoxic T cells throughout the tumor. These results qualitatively support the data from flow cytometry and qPCR, which suggest that cytotoxic immune cells are recruited to the tumor site after treatment with tAPC reprogramming NPs. Scale bar: 200 μm (A) or 100 μm (FIG. 29B-FIG. 29C);

FIG. 30 shows that the intrinsic immune state of tumor affects its susceptibility to immunotherapy. More immunogenic tumors like MC38 have higher levels of leukocyte infiltration in general than “cold” tumors like B16-F10, but treatment of “cold tumors” with tAPC nanoparticles in combination with checkpoint blockade can lead to greatly increased immune infiltration. Other specific immune populations also differ in the tumor types and may affect their immunogenicity. Other “cold” tumors like 4T1 may be affected by other immune populations like pro-tumorigenic neutrophils;

FIG. 31 shows C57BL/6 mice were injected with a subcutaneous flank tumor on t=0 days. After 7 days, GFP+ B16-F10 cells were injected intravenously into the same mice to form metastasis-like lesions in the lungs. At the same time point (t=7 days), mice were treated by intratumoral injection of control (fLuc) or tAPC (4-1BBL/IL-12) nanoparticles in the flank tumor as well as systemic (intraperitoneal) administration of anti-PD-1 therapy following the same dosing and schedule as in FIG. 6. At t=21 days (14 days after intravenous injection of tumor cells), mice were euthanized. The size of the flank tumor was measured and found to be statistically significantly lower in the treatment group than in the controls (p<0.001), even though the administration site was in the flank tumor, reflected in the changes in the immune state of the flank tumor after treatment. The amount of GFP+B16-F10 cells in the lungs was measured by flow cytometry and was found to be significantly lower in the tAPC-treated mice than in control mice (p<0.05). It should be noticed that four out of ten (40%) of the control mice died prior to flow cytometry analysis, and their lungs were qualitatively observed to be heavily colonized with B16-F10 tumor lesions; thus, it is expected that an even greater effect would have been measured if all mice had survived to the final time point at which quantitative measurements were taken;

FIG. 32 shows CT-2A mouse glioma tumors were injected subcutaneously and treated with tAPC nanoparticles or control nanoparticles into the tumor on days t=10, t=12, and t=14. No checkpoint blockade therapy was administered. Fifty percent (50%) of tumors treated with tAPC nanoparticles were cleared entirely, and those animals survived long-term; 100% of control animals died; and

FIG. 33 shows that signal 2 and signal 3 genes were cloned into the N1 plasmid backbone and used to transfect B16-F10 cells in combinations. The transfected B16-F10 cells were co-cultured with primary splenocytes from mice, and the IFN-γ secretion into the media was measured after 7 days of co-culture in order to find the combination of genes that caused the greatest immune stimulation. IL-12 and IL-23 were found to be the most effective overall, with various signal 2 genes being effective in combination with certain signal 3 genes, particularly OX40L and CD80.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. Gene Delivery Particles to Induce Tumor-Derived Antigen Presenting Cells

The presently disclosed subject matter provides an innovative therapeutic agent consisting of gene delivery nanoparticle formulations comprising polymers and plasmids that reprograms cancer cells to activate an immune response to attack themselves and other cancer cells. The presently disclosed formulations can be used to kill cancer cells and/or to reduce tumor size in various types of cancer.

To this end, the presently disclosed subject matter provides a platform technology for activating lymphocytes that is “off-the-shelf,” entirely synthetic, and biodegradable. The presently disclosed platform can potentially reduce the cost and complexity of immunotherapy and facilitate overcoming various regulatory hurdles. The presently disclosed methods and compositions could have a major impact on cancer patients, particularly those suffering from metastatic disease, and, more broadly, on the field of cancer immunotherapy as a whole.

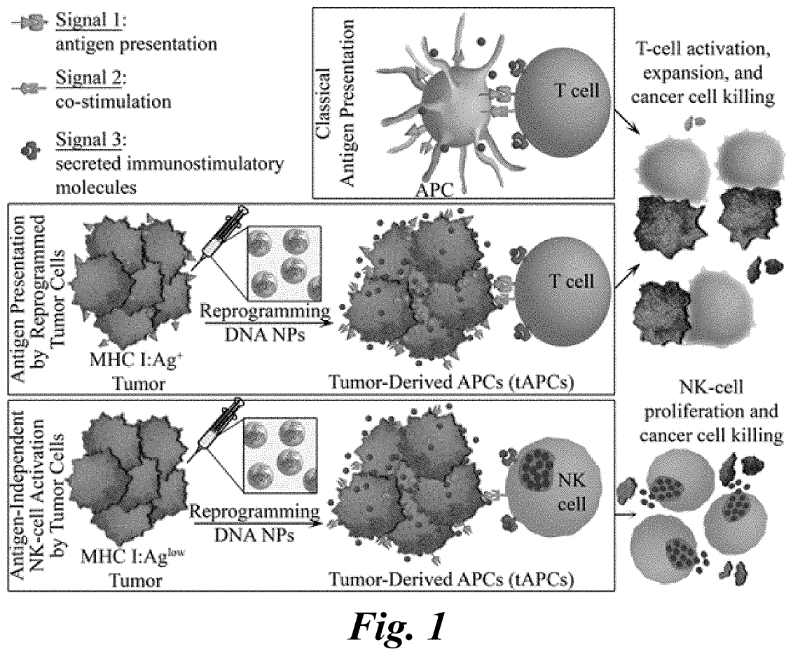

More particularly, the presently disclosed subject matter uses synthetic, biodegradable nanoparticles (NPs) to reprogram tumor cells into “tumor-derived APCs” (referred to herein as “tAPCs”) in vivo to activate T cells and natural killer (NK) cells for systemic tumor rejection (see FIG. 1). Antigen-presenting cells (APCs) activate T-cells by presenting coordinated signals, including antigen (signal 1); surface-bound co-stimulatory molecules (signal 2); and secreted cytokines (signal 3). Many tumor cells already express signal 1 (tumor antigen in the context of major histocompatibility complex class I (MHC I), see Comber and Philip, 2014, and, without wishing to be bound to any one particular theory, it is thought that tumor cells can be programmed to express the other signals necessary for them to act as APCs, directing cytotoxic T-cell responses against themselves and other tumor cells.

Further, delivery of soluble signals by the presently disclosed method can be used to increase signal 1 expression, further increasing the immunogenicity of the tAPCs. This aspect enables an antigen-agnostic therapy that elicits a systemic immune response targeting multiple antigens expressed by the tumor cells at the time of treatment. As an additional advantage, even should MHC I expression be downregulated in tumor cells, the presently disclosed strategy would result in presentation of activating signals to NK cells, which often have been implicated in tumor control in cases of successful immunotherapy.

The combination of both signal 2 and signal 3 is crucial, as the soluble signal 3 allows for local recruitment of cells and affects their cell fate, while signal 2 expression on cancer cells causes activation of immune cells directly against cancer. While others have delivered or expressed soluble cytokines (signal 3), see, for example, Emtage et al., 1999; Narvaiza et al., 2000; and Nomura et al., 2001, and/or adjuvants, see Fan et al., 2017; Hanson et al., 2015, systemically or locally, the presently disclosed method is distinct: by expressing signal 2 and signal 3 on signal 1-bearing tumor cells, T cells can be directly activated in the context of the tumor antigen, leading to an antigen-specific cellular response despite the antigen-free non-cellular approach. The local expression of these immune-stimulatory molecules is crucial, as systemic delivery of cytokines and signal 2 agonists can lead to unacceptable levels of adverse side effects. See Lasek et al., 2014; Di Giacomo et al., 2010; and Leonard et al., 1997.

Local gene delivery of cytokines and overexpression of co-stimulatory signal 2 molecules by tumor cells themselves is a promising strategy by which to address this issue. To this end, the presently disclosed subject matter provides a non-viral method of transfecting surface-bound signal 2 and secreted signal 3 together into a tumor mass, allowing the challenges and risks of traditional virus-associated gene delivery to be evaded, including the development of immunity to the viral vector itself.

Accordingly, the presently disclosed subject matter provides nanoparticle formulations and methods of their use for inducing tumor cells to express co-stimulatory molecules and cytokines for T-cell and NK cell activation. The presently disclosed cationic nanoparticles can form nanoplexes with negatively charged cargo, e.g., nucleic acids, via electrostatic interactions. In some embodiments, the presently disclosed subject matter provides a composition comprising:

(a) a genetic element that encodes a “Signal 2” (e.g., a cell surface bound protein that regulates immune cells, such as 4-1BBL, CD80, CD86, and OX40L);

(b) a genetic element that encodes a “Signal 3” (e.g., a secreted protein that regulates immune cells, such as IL-2, IL-12, IL-6, IL-7, IL-15, IL-18, IL-21, IFN-α, and IFN-β); and

(c) a cationic biomaterial or biomaterial blend that encapsulates elements (a) and (b) into a nano-scale particle (e.g., a particle having a size, for example, a diameter or other dimension, ranging from about 20 nm to about 500 nm).

In some embodiments, the cationic biomaterial or biomaterial blend comprises a cationic polymer. In some embodiments, the cationic biomaterial or biomaterial blend comprises a cationic biodegradable polymer(s). In particular embodiments, the cationic biodegradable polymer is selected from individual polymers or blends from the group consisting of: poly(lactic-co-glycolic acid) (PLGA), polycaprolactone (PCL), polyglycolide (PGA), poly(lactic acid) (PLA), a polyhydroxyalkanoate (PHA), such as poly-3-hydroxybutyrate (P3HB), poly(acrylic acid) (PAA), poly(3-hydroxybutyrate-co-3-hydroxyvalerate) (PHBV), a poly(beta-amino ester) (PBAE), or combinations thereof, or other hydrolytically biodegradable polymers.

Accordingly, the presently disclosed biodegradable particles include one or more of the following biodegradable polymers:

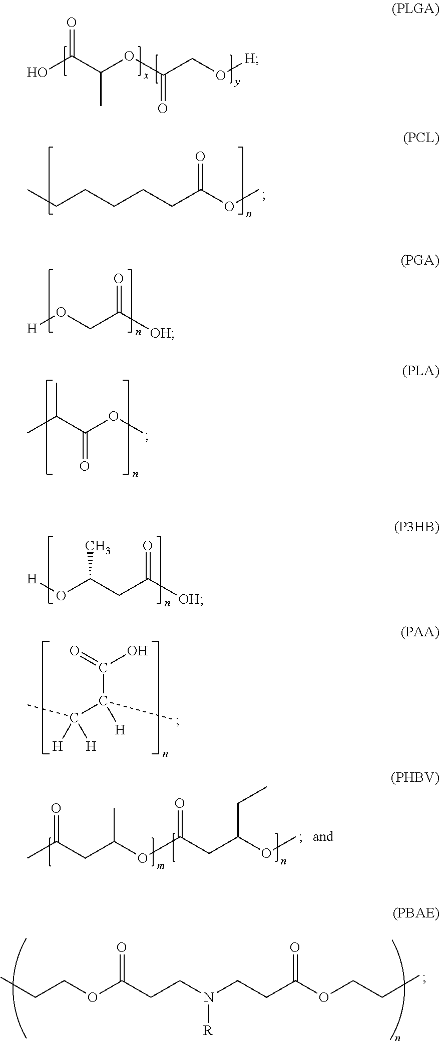

wherein each x, y, m, and n can independently be an integer from 1 to 10,000.

As used herein, “biodegradable” polymers and/or nanoparticles are those that, when introduced into cells, are broken down by the cellular machinery or by hydrolysis into components that the cells can either reuse or dispose of without significant toxic effect on the cells (i.e., fewer than about 20% of the cells are killed when the components are added to cells in vitro). Such components preferably do not induce inflammation or other adverse effects in vivo. In certain preferred embodiments, the chemical reactions relied upon to break down the biodegradable compounds are uncatalyzed.

In certain embodiments, the biodegradable nanoparticles comprise a chemical moiety having one or more degradable linkages, such as an ester linkage, a disulfide linkage, an amide linkage, an anhydride linkage, and a linkage susceptible to enzymatic degradation. Representative degradable linkages include, but are not limited to:

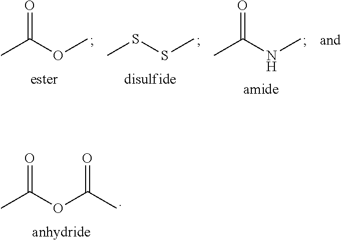

In some embodiments, the biodegradable particle comprises a poly(lactic-co-glycolic acid) polyethylene glycol (PLGA-PEG) block copolymer. In other embodiments, the biodegradable particle comprises a poly(lactic acid)-based polymeric matrices, such as polylactic acid (PLA), poly(D,L-lactide-co-glycolide) (PLGA), and poly (D,L-lactic acid) (PDLLA). In other embodiments, the biodegradable particle comprises a copolymer of a poly(lactic acid)-based polymer and a non-poly(lactic acid)-based polymer, such as a combination of PLA and PCL. In some embodiments, blends of polyesters may be used, such as PLGA/PCL or PLGA/PBAE. In some embodiments, the PLGA content is between about 50 to about 90% with the remainder being PCL and/or PBAE. In particular embodiments, the biodegradable particle comprises a blend of PLGA and a (PBAE). In yet other embodiments, nondegradable polymers that are used in the art, such as polystyrene, are blended with a degradable polymer or polymers disclosed immediately hereinabove to create a copolymer system. Accordingly, in some embodiments, a nondegradable polymer is blended with the biodegradable polymer.

In some embodiments, the cationic biomaterial or biomaterial blend comprises a poly(beta-amino ester)(s) (PBAEs). Exemplary PBAEs suitable for use with the presently disclosed subject matter include those disclosed in:

U.S. Pat. No. 9,884,118 for Multicomponent Degradable Cationic Polymers, to Green et al., issued Feb. 6, 2018;

U.S. Pat. No. 9,802,984 for Biomimetic Peptide and Biodegradable Delivery Platform for the Treatment of Angiogenesis- and Lymphangiogenesis-Dependent

Diseases, to Popel et al., issued Oct. 31, 2017;

U.S. Pat. No. 9,717,694 for Peptide/Particle Delivery Systems, to Green et al., issued Aug. 1, 2017;

U.S. Pat. No. 8,992,991 for Multicomponent Degradable Cationic Polymers, to Green et al., issued Mar. 31, 2015;

U.S. Patent Application Publication No. 20180256745 for Biomimetic Artificial Cells: Anisotropic Supported Lipid Bilayers on Biodegradable Micro and Nanoparticles for Spatially Dynamic Surface Biomolecule Presentation, to Meyer et al., published Sep. 13, 2018;

U.S. Patent Application Publication No. 20180112038 for Poly(Beta-Amino Ester)-Co-Polyethylene Glycol (PEG-PBAE-PEG) Polymers for Gene and Drug Delivery, to Green et al., published Apr. 26, 2018;

U.S. Patent Application Publication No. 20170216363 for Nanoparticle Modification of Human Adipose-Derived Mesenchymal Stem Cells for Treating Brain Cancer and other Neurological Diseases, to Quinones-Hinojosa and Green, published Aug. 3, 2017;

U.S. Patent Application Publication No. 20150273071 for Bioreducible Poly (Beta-Amino Ester)s For siRNA Delivery, to Green et al., published Oct. 1, 2015;

U.S. Pat. No. 8,287,849 for Biodegradable Poly(beta-amino esters) and Uses Thereof, to Langer, et al., issued Oct. 16, 2012;

each of which is incorporated by reference in their entirety.

The presently disclosed multicomponent degradable cationic polymers can be prepared by the following reaction scheme:

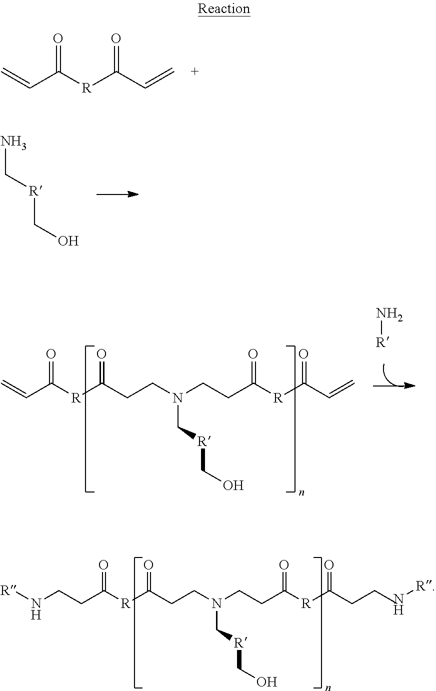

Generally, the presently disclosed multicomponent degradable cationic polymers include a backbone derived from a diacrylate monomer (designated herein below as “B”), an amino-alcohol side chain monomer (designated herein below as “S”), and an amine-containing end-cap monomer (designated herein below as “E”). The end group structures are distinct and separate from the polymer backbone structures and the side chain structures of the intermediate precursor molecule for a given polymeric material. The presently disclosed PBAE compositions can be designated, for example, as B5-S4-E7 or 547, in which R is B5, R′ is S4, and R″ is E7, and the like, where B is for backbone and S is for the side chain, followed by the number of carbons in their hydrocarbon chain. Endcapping monomers, E, are sequentially numbered according to similarities in their amine structures.

The polymer backbone can comprise a diacrylate having the following general formula, where Rₒ comprises a linear, branched, and/or substituted alkylene, and may comprise one or more heteroatoms, such as O, N, or S, and may include one or more carbocyclic, heterocyclic, and aromatic groups:

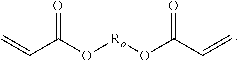

In some embodiments, the diacrylate has the general formula of:

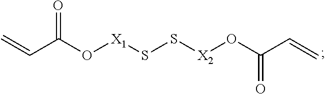

where X₁ and X₂ are each independently C₁-C₃₀ alkylene chains.

In particular embodiments, the diacrylate monomer for the polymer backbone is selected from:

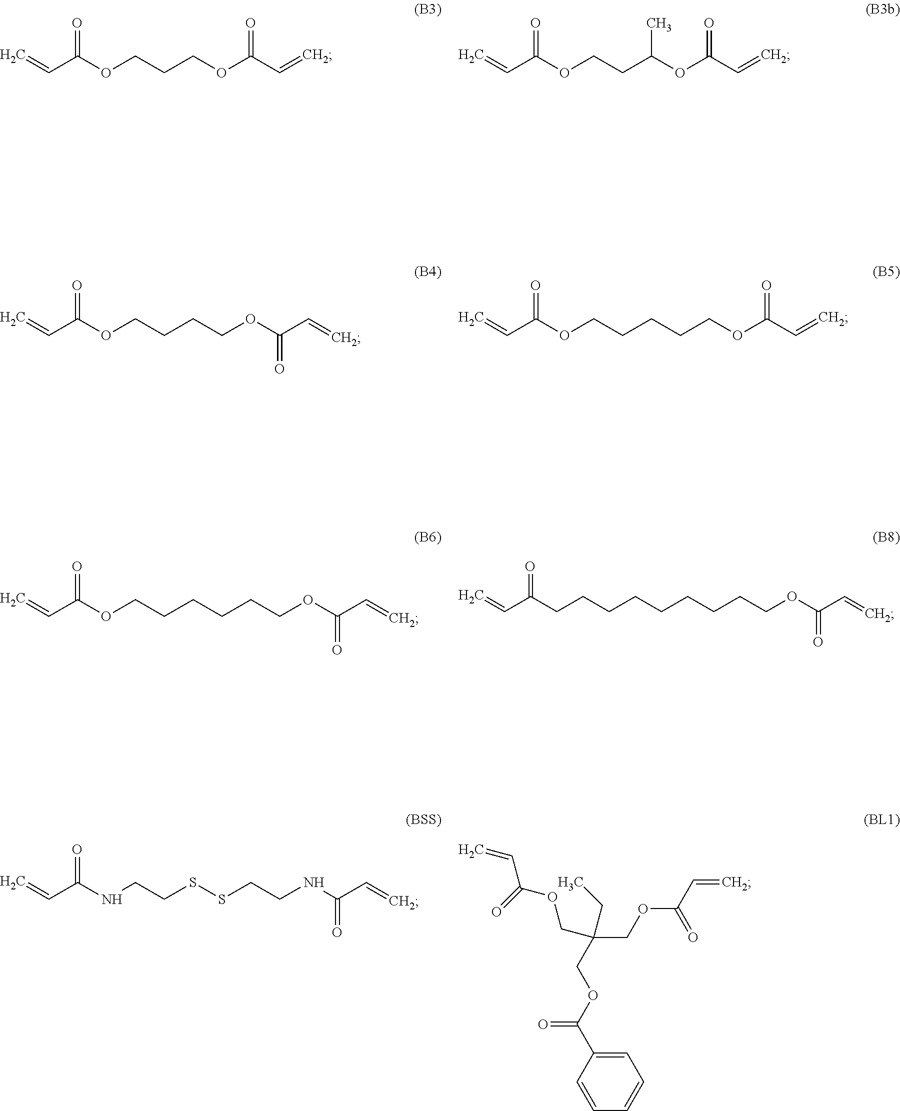

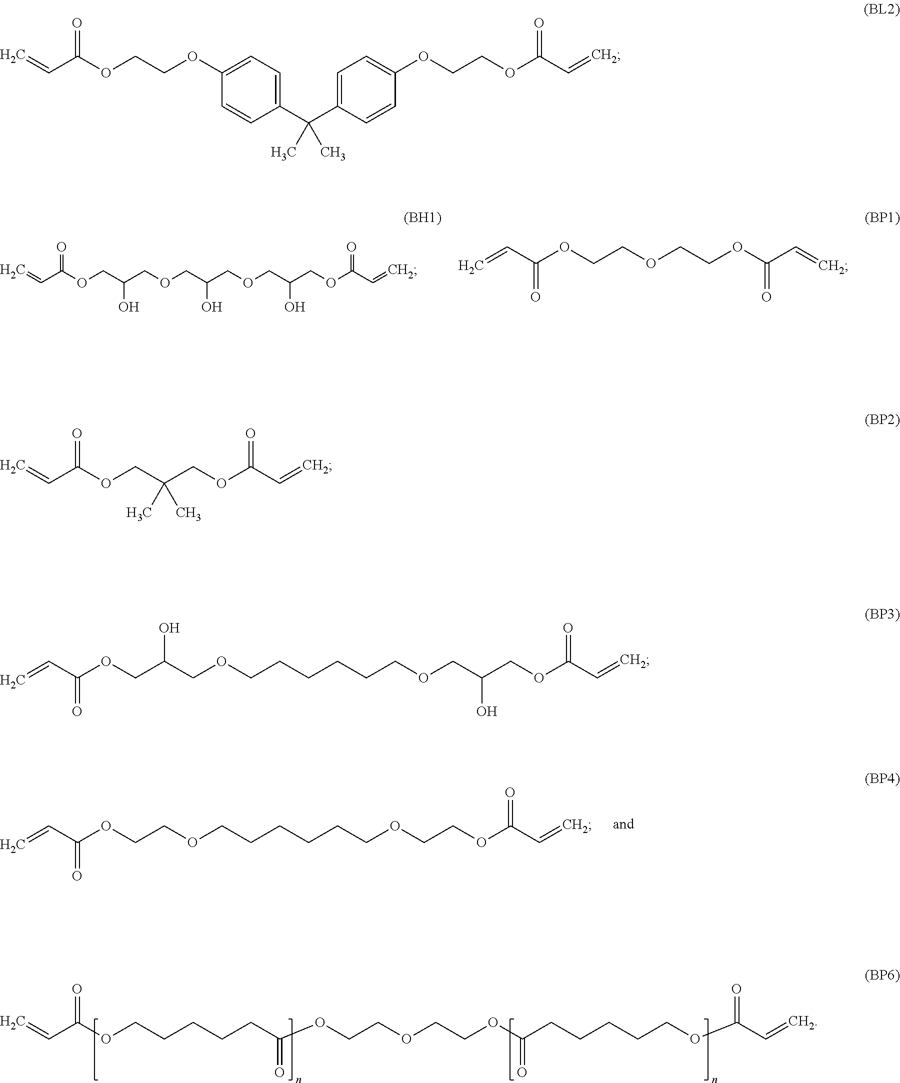

As shown in the reaction scheme provided hereinabove, acrylate monomers can be condensed with amine-containing side chain monomers. In some embodiments, the side chain monomers comprise a primary amine, but, in other embodiments, comprise secondary and tertiary amines. Side chain monomers may further comprise a C₁ to C₈ linear or branched alkylene, which is optionally substituted. Illustrative substituents include hydroxyl, alkyl, alkenyl, thiol, amine, carbonyl, and halogen.

In particular embodiments, the side chain monomer is selected from:

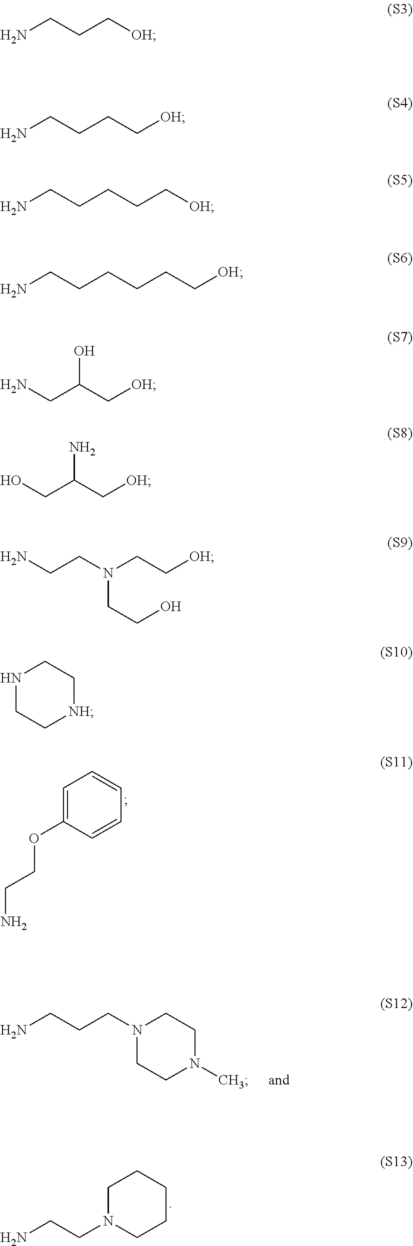

The PBAE polymer further comprises an end group, which may include one or more primary, secondary or tertiary amines, and may include aromatic and non-aromatic carbocyclic and heterocyclic groups, such as carbocyclic and heterocyclic groups of 5 or 6 atoms. The end group in some embodiments may comprise one or more ether, thioether, or disulfide linkages.

Representative end groups include, but are not limited to:

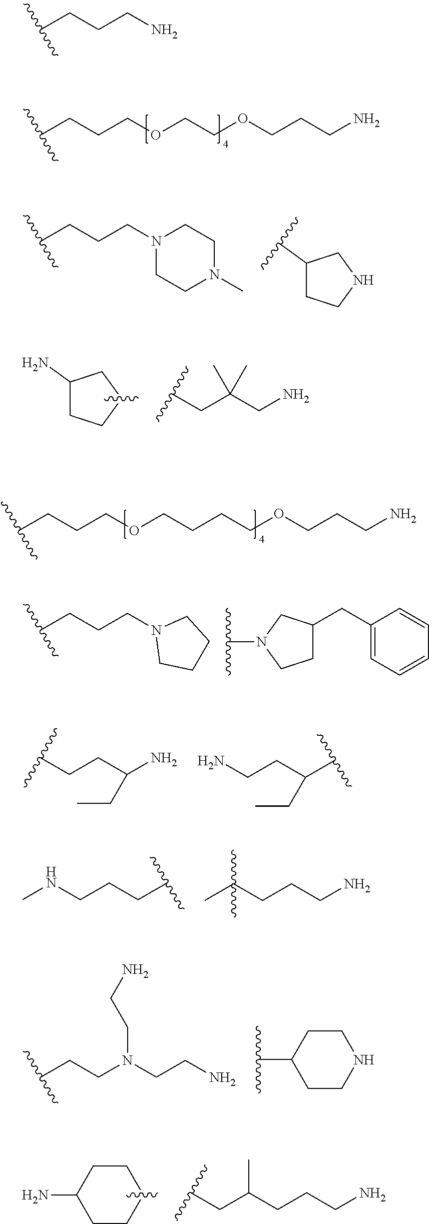

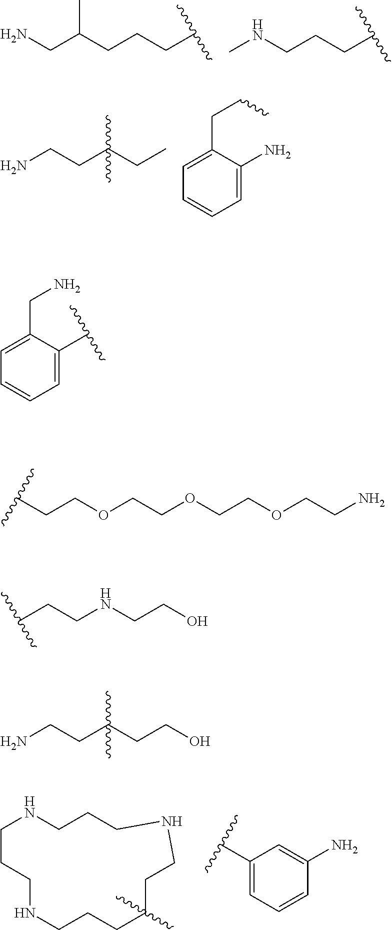

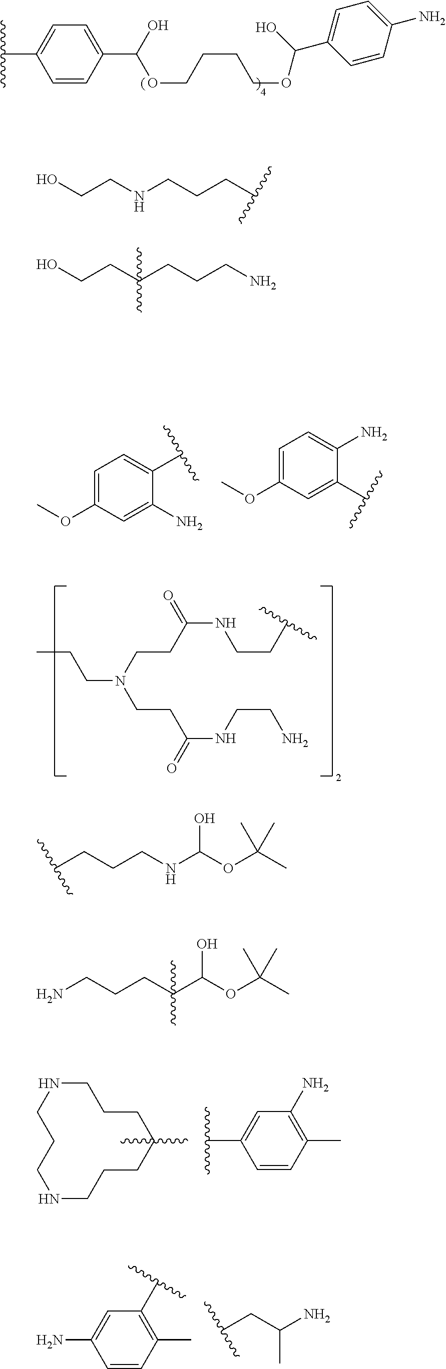

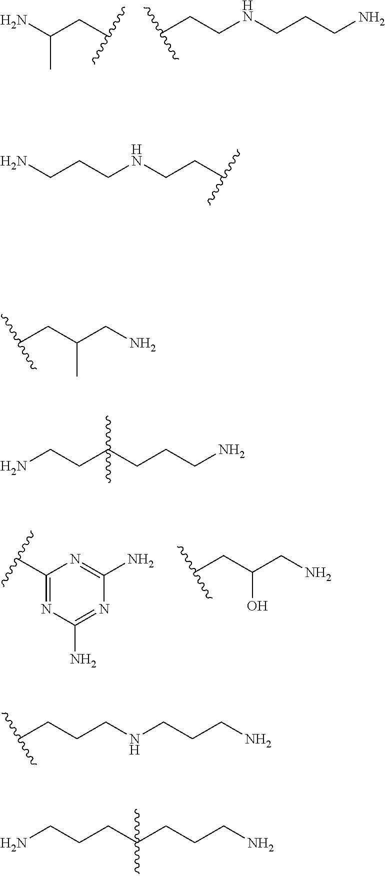

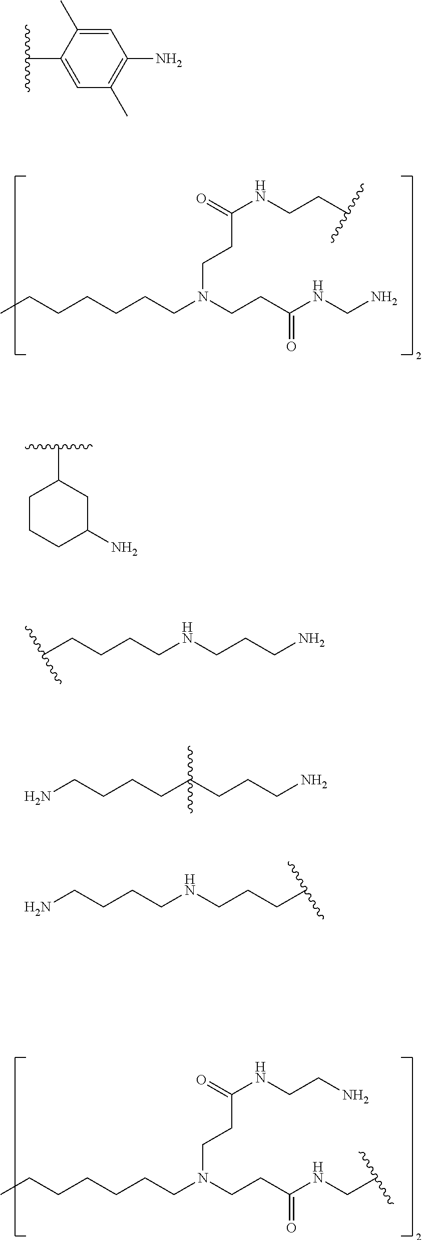

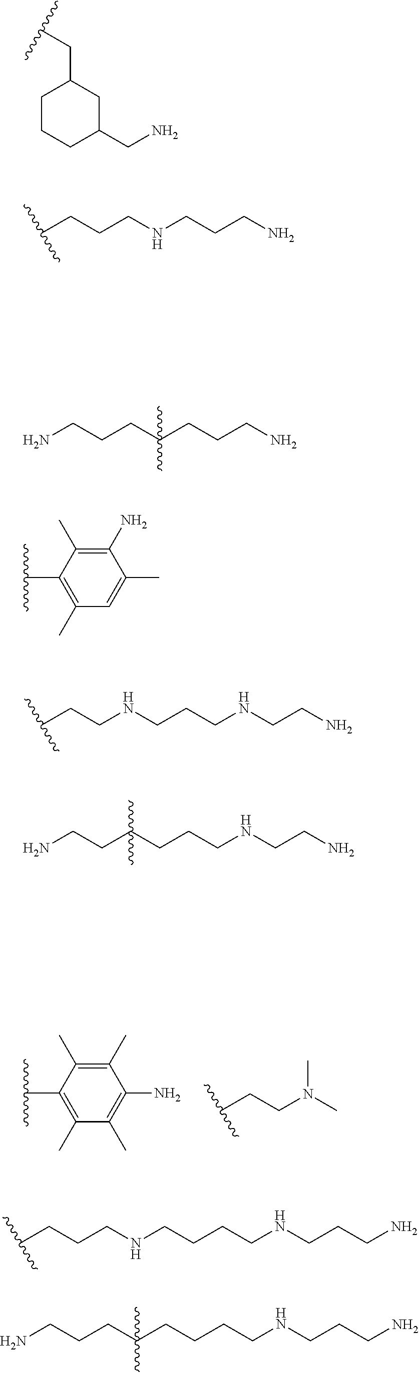

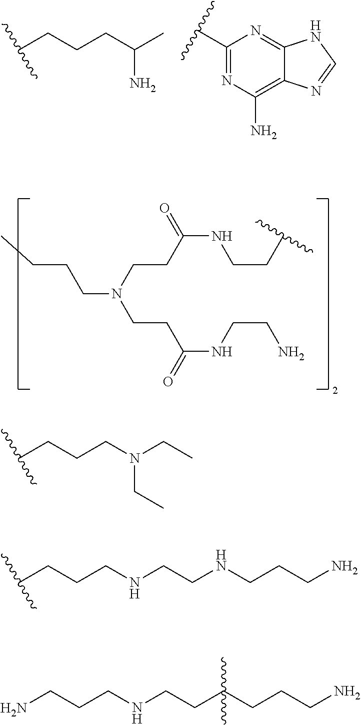

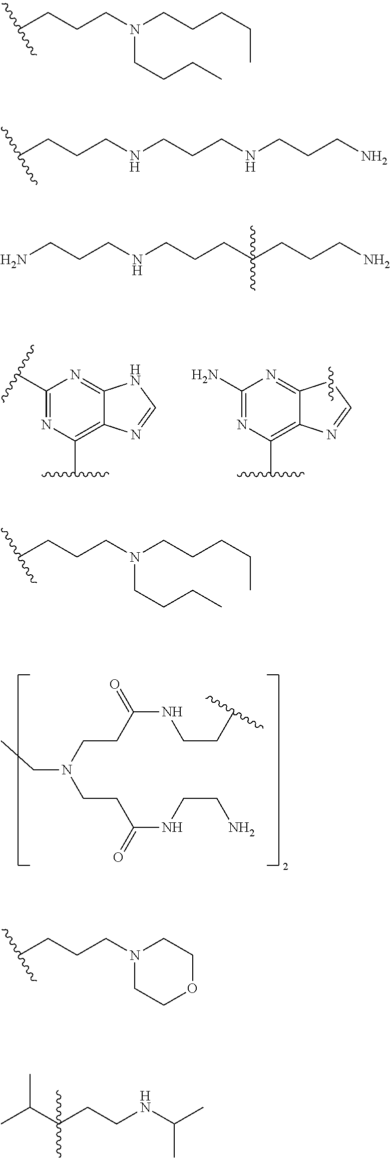

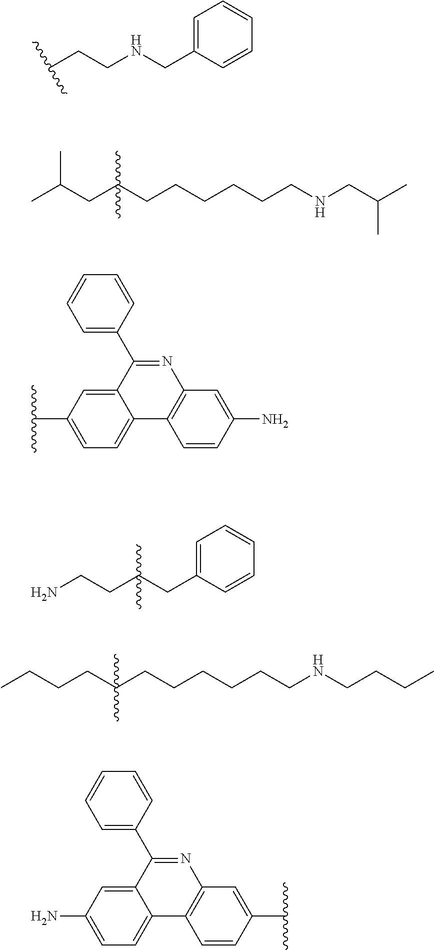

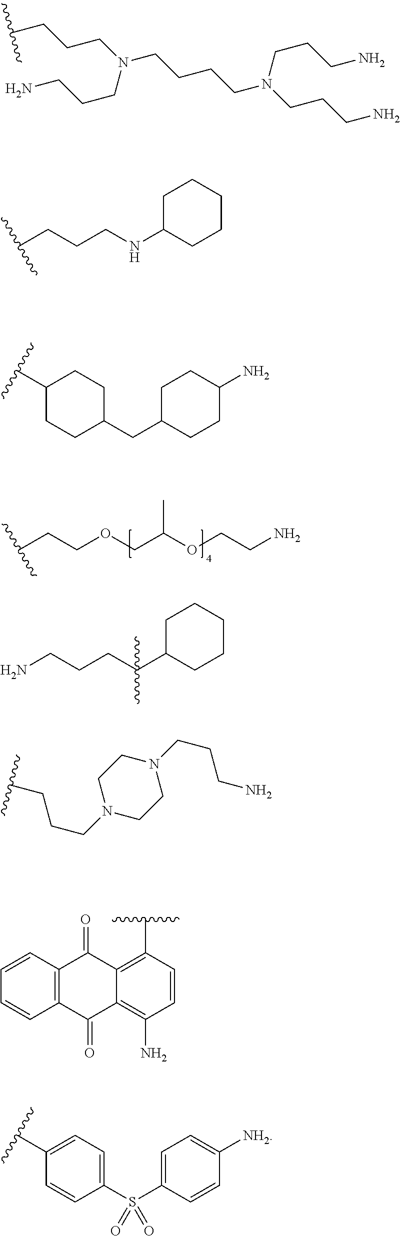

In other embodiments, the end group monomer is selected from the group consisting of:

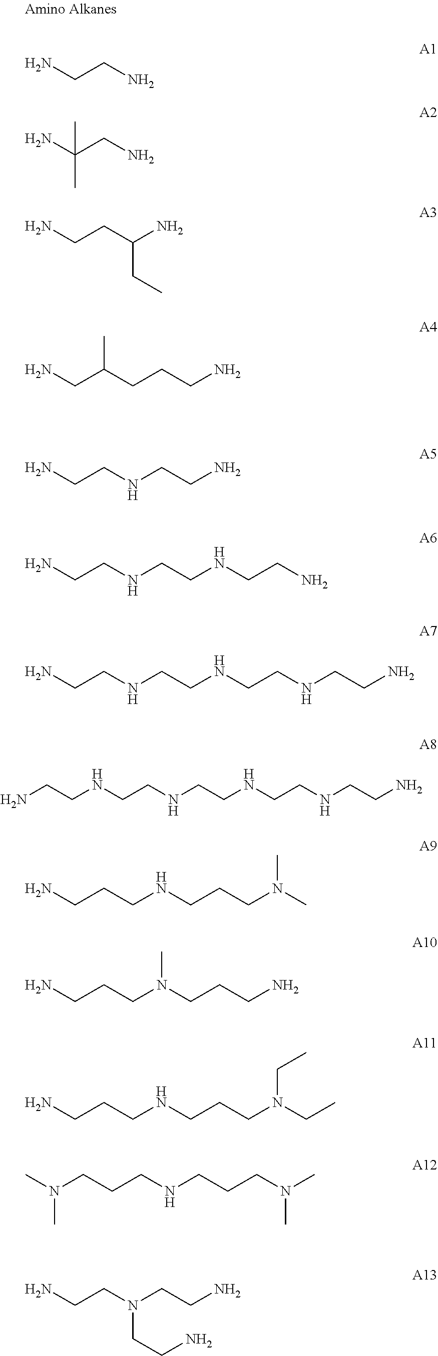

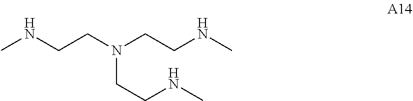

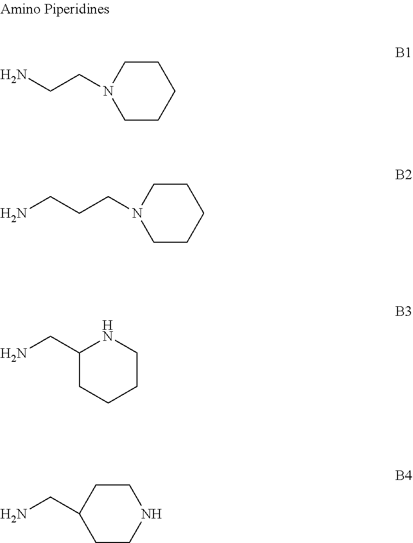 [SMILES: NCCN1CCCCC1 ; NCCCN1CCCCC1 ; NCC1CCCCN1 ; NCC1CCNCC1]

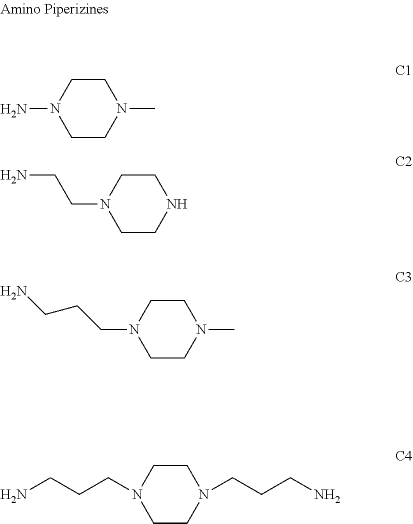

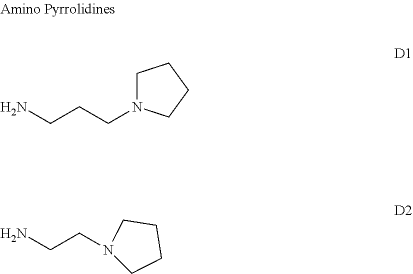 [SMILES: NCCCN1CCCC1 ; NCCN1CCCC1]

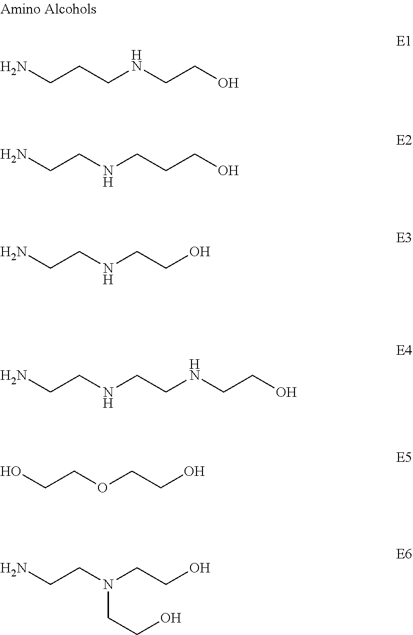 [SMILES: NCCCNCCO ; NCCNCCCO ; NCCNCCO ; NCCNCCNCCO ; OCCOCCO ; NCCN(CCO)CCO]

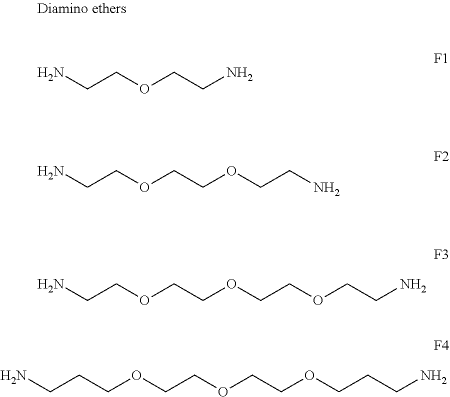 [SMILES: NCCOCCN ; NCCOCCOCCN ; NCCOCCOCCOCCN ; NCCCOCCOCCOCCCN]

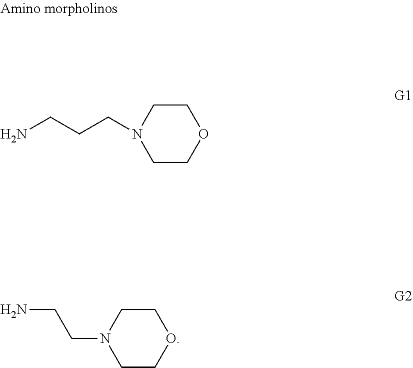 [SMILES: NCCCN1CCOCC1 ; NCCN1CCOCC1]

In yet other embodiments, the end group monomer selected from the group consisting of:

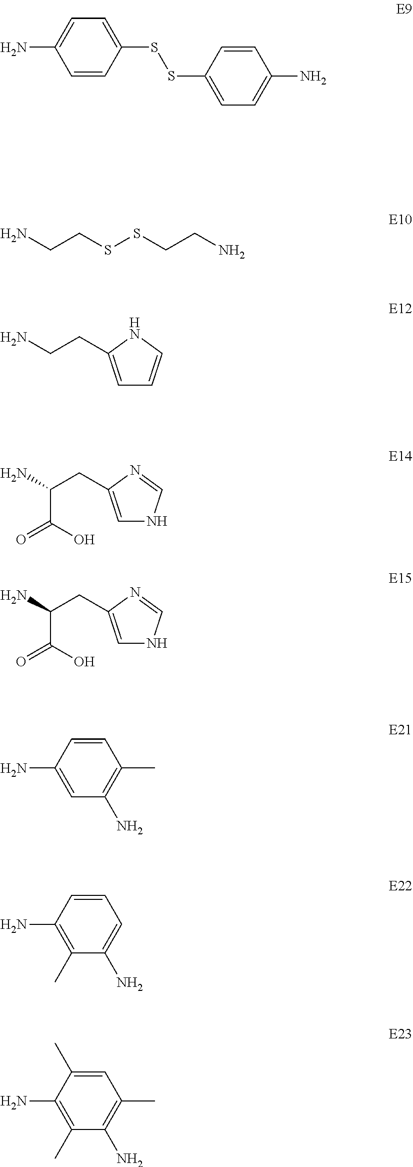

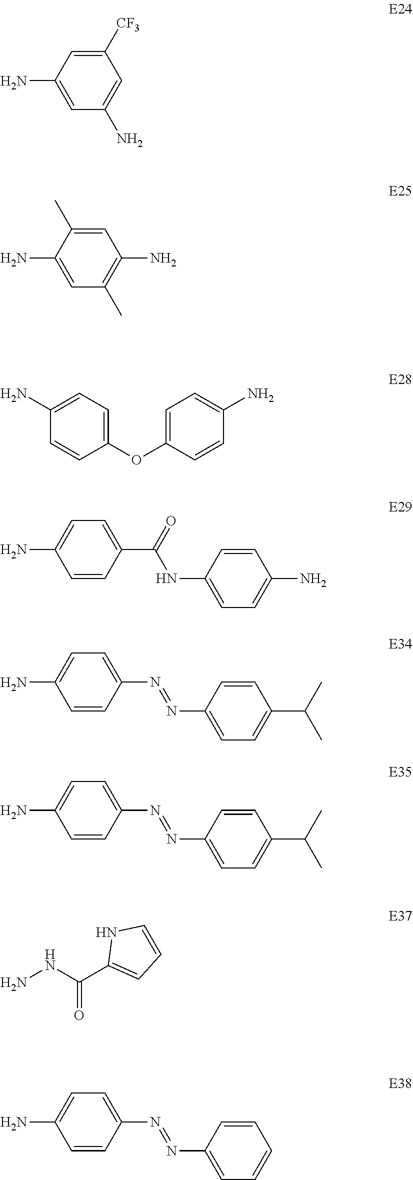

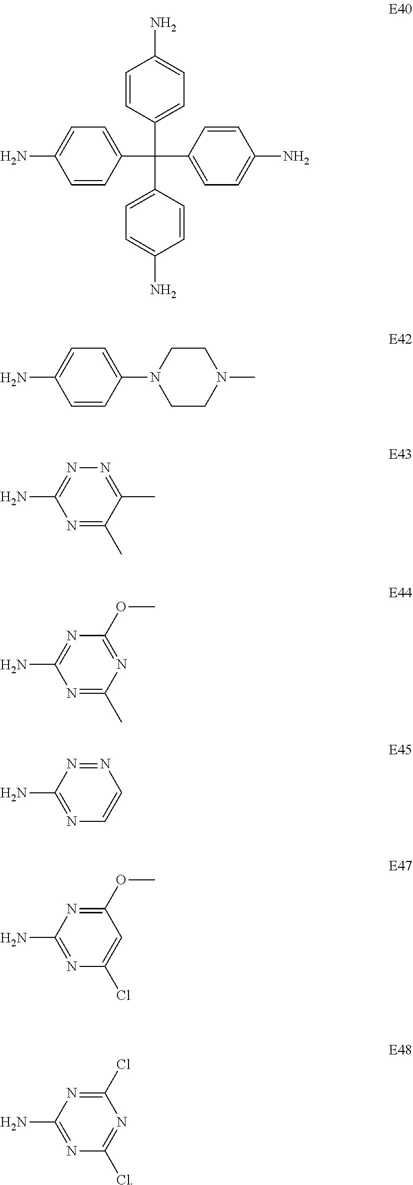

Further, Table 1 presents, in more detail, particular monomers used for PBAE library synthesis. Acrylate terminated polymers were synthesized from small molecule diacrylate and primary amine monomers followed by high-throughput endcapping with 37 monomers organized into different structural categories.

| | Monomer | Internal | MW | |
|---|---|---|---|---|
| Chemical Name | Name | Name | (Da) | CAS number |
| Base-Monomers (B) | | | | |
| 1,3-propanediol diacrylate | B3 | B3 | 184.19 | 24493-53-6 |
| 1,4-Butanediol diacrylate | B4 | B4 | 198.22 | 1070-70-8 |
| 1,5-Pentanediol diacrylate | B5 | B5 | 212.24 | 36840-85-4 |
| Side-chain-Monomers (S) | | | | |
| 3-amino-1-propanol | S3 | S3 | 75.11 | 156-87-6 |
| 4-amino-1-butanol | S4 | S4 | 89.14 | 13325-10-05 |
| 5-amino-l-pentanol | S5 | S5 | 103.16 | 2508-29-4 |
| Endcap Monomers | | | | |
| 1,3-diaminopropane | A1 | E1 | 74.12 | 109-76-2 |
| 2,2-dimethyl-1,3-propanediamine | A2 | E2 | 102.18 | 7328-91-8 |
| 1,3-diaminopentane | A3 | E3 | 102.18 | 589-37-7 |
| 2-methyl-1,5-diaminopentane | A4 | E4 | 116.2 | 15520-10-02 |
| Diethylentramine | A5 | E63 | 103.17 | 111-40-0 |
| Triethylenetetramine | A6 | E30 | 146.23 | 112-24-3 |
| Tetraethylenepentamine | A7 | E31 | 189.3 | 1112-57-2 |
| Pentaethylenehexamine | A8 | E60 | 232.44 | 4067-16-7 |
| N,N-Dimethyldipropylenetriamine | A9 | E49 | 159.27 | 10563-29-8 |
| 3,3′-Diamino-N-methyldipropylamine | A10 | E52 | 145.25 | 105-33-9 |
| N,N-Diethyldiethylenetriamine | A11 | E58 | 159.27 | 24426-16-2 |
| 3,3′-Iminobis(N,N-dimethylpropylamine) | A12 | E56 | 187.33 | 6711484 |
| Tris(2-aminoethyl)amine | A13 | E32 | 146.23 | 4097-89-6 |
| Tris[2-(methylamino)ethyl]amine | A14 | E54 | 188.31 | 65604-89-9 |
| 1-(2-Aminoethyl)piperidine | B1 | E53 | 128.22 | 27578-60-5 |
| N-(3-Aminopropyl)piperidine | B2 | E61 | 128.22 | 3529-08-6 |
| 2-(Aminomethyl)piperidine | B3 | E50 | 114.19 | 22990-77-8 |
| 4-(Aminomethyl)piperidine | B4 | E64 | 114.19 | 7144-05-0 |
| 1-Amino-4-methylpiperazine | Cl | E40 | 115.18 | 6928-85-4 |
| 1-(2-Aminoethyl)piperazine | C2 | E39 | 129.2 | 140-31-8 |
| 1-(3-Aminopropy1)-4- | C3 | E7 | 157.26 | 4572-031 |
| methylpiperazine | | | | |
| 1,4-Bis(3-aminopropyl)piperazine | C4 | E65 | 200.33 | 7209-38-3 |
| 1-(3-Aminopropyl)pyrrolidine | D1 | E8 | 128.22 | 23159-07-01 |
| 1-(2-Aminoethyl)pyrrolidine | D2 | E59 | 114.19 | 7154-73-6 |
| 2-(3-Aminopropylamino)ethanol | E1 | E6 | 118.18 | 4461-39-6 |
| N-(3-Hydroxypropyl) | E2 | E51 | 118.18 | 56344-32-2 |
| ethylenediamine | | | | |
| N-(2-Hydroxyethyl) | E3 | E62 | 104.15 | 111-41-1 |
| ethylenediamine | | | | |
| N,N′-Bis(2-hydroxyethyl) | E4 | E16 | 148.2 | 4439-20-7 |
| ethylenediamine | | | | |
| 2-(2-Aminoethoxy)ethanol | E5 | E55 | 105.14 | 929-06-6 |
| N,N-Bis(2- | E6 | E18 | 148.2 | 3197-06-6 |
| hydroxyethyl)ethylenediamine | | | | |
| 2,2′-Oxybis(ethylamine) | F1 | E57 | 104.15 | 2752-17-2 |
| 2,2′-(Ethylenedioxy)bis | F2 | E33 | 148.2 | 929-59-9 |
| (ethylamine) | | | | |
| 1,11-diamino-3,6,9-trioxaundecane | F3 | E5 | 192.26 | 929-75-9 |
| 4,7,10-Trioxa-1,13- | F4 | E27 | 220.31 | 4246-51-9 |
| tridecanediamine | | | | |
| 3-Morpholinopropylamine | G1 | E89 | 144.21 | 123-00-2 |
| 4-(2-Aminoethyl)morpholine | G2 | E90 | 130.19 | 2038-031 |

TABLE 2
presents additional endcap monomers suitable for use with
the presently disclosed PBAEs.

| | Monomer | | CAS |
|---|---|---|---|
| Chemical Name | Name | MW | number |
| Minimally effective | | | |
| endcap monomers | | | |
| 4-Aminophenyl disulfide | E9 | 248.37 | 722-27-0 |
| Cystamine dihydrochloride | E10 | 250.2 | 56-17-7 |
| Histamine | E12 | 111.15 | 51-45-6 |
| D-Histidine | E14 | 155.15 | 351-50-8 |
| L-Histidine | E15 | 155.15 | 71-00-1 |
| 2,4-Diaminotoluene | E21 | 122.17 | 95-80-7 |
| 2,6-Diaminotoluene | E22 | 122.17 | 823-40-5 |
| 2,4,6-Trimethyl-phenylenediamine | E23 | 150.22 | 3102-70-3 |
| 5-(Trifluoromethyl)-1,3- | E24 | 176.14 | 368-53-6 |
| phenylenediamine | | | |
| p-Phenylenediamine | E25 | 108.14 | 106-50-3 |
| 2,5-Dimethy1-1,4-phenylenediamine | E26 | 136.19 | 6393-01-7 |
| 4,4′-Oxydianiline | E28 | 200.24 | 101-80-4 |
| 4-Diaminobenzanilide | E29 | 227.26 | 785-30-8 |
| N,N-Dimethyl-4,4'-azodianiline | E34 | 240.3 | 539-17-3 |
| 4-[(E)-(4-aminophenyl) | E35 | 212.256 | — |
| diazenyl]phenylamine | | | |
| 1H-pyrrole-2-carbohydrazide | E37 | 125.13 | 50269-95-9 |
| 4-Aminoazobenzene | E38 | 197.24 | 60-09-3 |
| Tetrakis(4-aminophenyl)methane | E40 | 380.48 | 60532-63-0 |
| 1-(4-Aminophenyl)piperazine | E42 | 177.25 | 67455-41-8 |
| 3-Amino-5,6-dimethy1-1,2,4-triazine | E43 | 124.14 | 17584-12-2 |
| 2-Amino-4-methoxy-6-methyl- | E44 | 140.14 | 1668-54-8 |
| 1,3,5-triazine | | | |
| 3-Amino-1,2,4-triazine | E45 | 96.09 | 1120-99-6 |
| 2-Amino-4-chloro-6- | E47 | 130.19 | 5734-64-5 |
| methoxypyrimidine | | | |
| 2-Amino-4,6-dichloro- | E48 | 164.98 | 933-20-0 |
| 1,3,5-triazine | | | |

In particular embodiments, the PBAE is constructed with an end group monomer selected from:

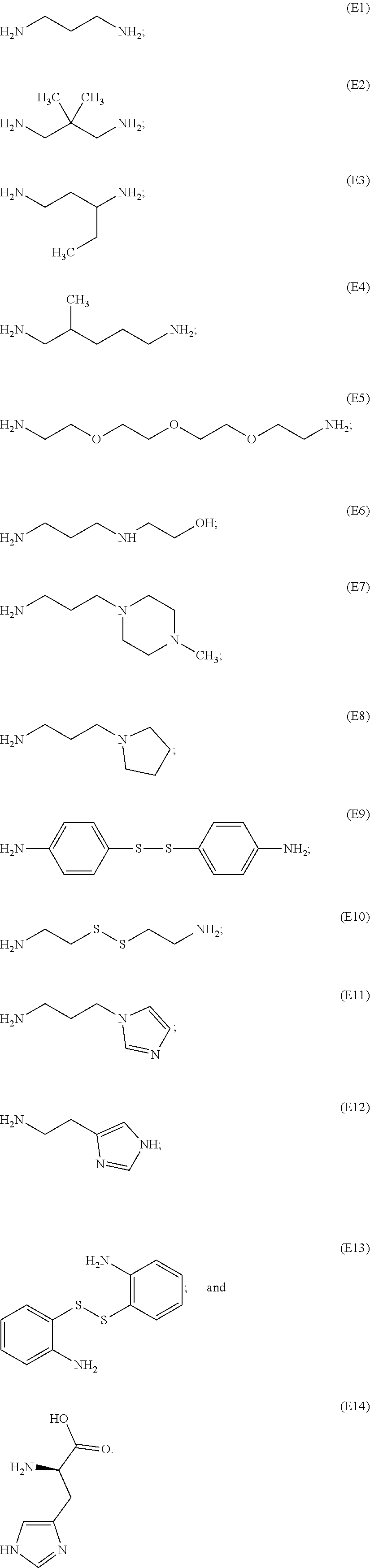

In even more particular embodiments, a combination of R, R′, and R″ is selected from the group consisting of:

| Compound | | |
|---|---|---|
| Code | R | R′ |
| 4-4-6 | 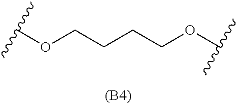 | 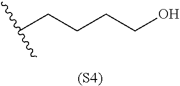 [SMILES: *CCCCO ; "(S4)"] |
| 4-4-7 | 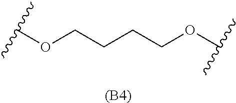 | 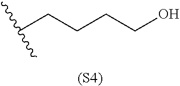 [SMILES: *CCCCO ; "(S4)"] |
| 4-4-27 | 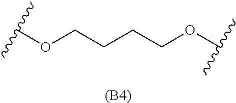 | 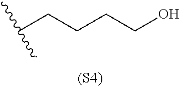 [SMILES: *CCCCO ; "(S4)"] |
| 4-5-3 | 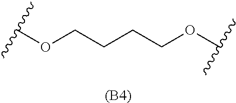 | 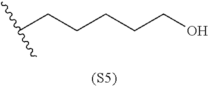 [SMILES: *CCCCCO ; "(S5)"] |
| 4-5-4 | 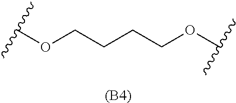 | 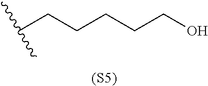 [SMILES: *CCCCCO ; "(S5)"] |
| 4-5-6 | 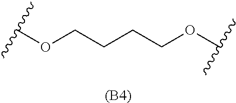 | 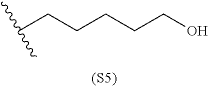 [SMILES: *CCCCCO ; "(S5)"] |
| 4-5-7 | 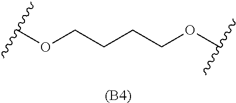 | 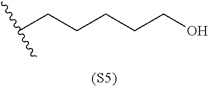 [SMILES: *CCCCCO ; "(S5)"] |
| 5-3-4 | 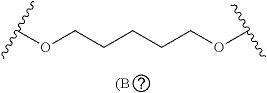 | 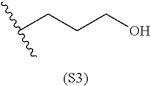 [SMILES: *CCCO ; "(S3)"] |
| 5-3-6 | 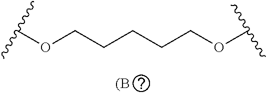 | 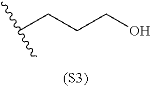 [SMILES: *CCCO ; "(S3)"] |
| 5-3-7 | 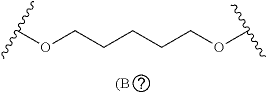 | 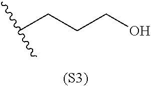 [SMILES: *CCCO ; "(S3)"] |
| 5-3-49 | 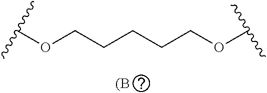 | 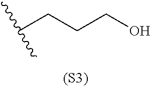 [SMILES: *CCCO ; "(S3)"] |
| 5-4-3 | 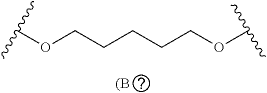 | 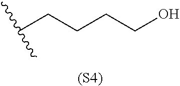 [SMILES: *CCCCO ; "(S4)"] |
| 5-4-4 | 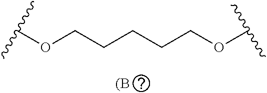 | 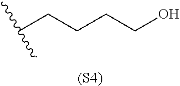 [SMILES: *CCCCO ; "(S4)"] |
| 5-4-6 | 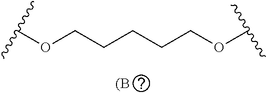 | 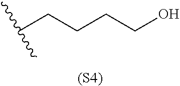 [SMILES: *CCCCO ; "(S4)"] |
| 5-4-7 | 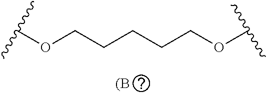 | 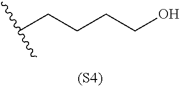 [SMILES: *CCCCO ; "(S4)"] |

| Compound | |
|---|---|
| Code | R″ |
| 4-4-6 | 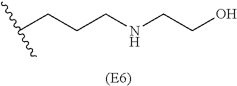 [SMILES: *CCCNCCO ; "(E6)"] |
| 4-4-7 | 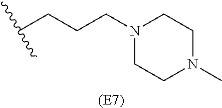 [SMILES: *CCCN1CCN(C)CC1 ; "(E7)"] |
| 4-4-27 | 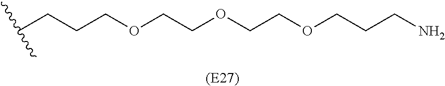 [SMILES: *CCCOCCOCCOCCCN ; "(E27)"] |
| 4-5-3 | 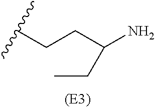 [SMILES: *CCC(N)CC] |
| 4-5-4 | 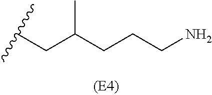 [SMILES: *CC(C)CCCN] |
| 4-5-6 | 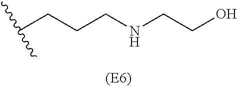 [SMILES: *CCCNCCO ; "(E6)"] |
| 4-5-7 | 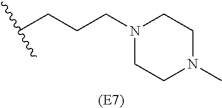 [SMILES: *CCCN1CCN(C)CC1 ; "(E7)"] |
| 5-3-4 | 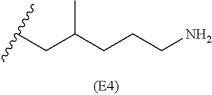 [SMILES: *CC(C)CCCN] |
| 5-3-6 | 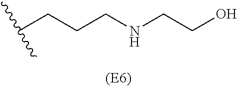 [SMILES: *CCCNCCO ; "(E6)"] |
| 5-3-7 | 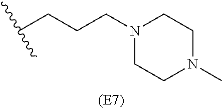 [SMILES: *CCCN1CCN(C)CC1 ; "(E7)"] |
| 5-3-49 | 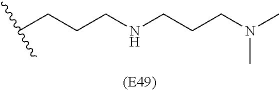 [SMILES: *CCCNCCCN(C)C ; "(E49)"] |
| 5-4-3 | 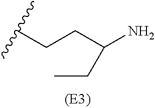 [SMILES: *CCC(N)CC] |
| 5-4-4 | 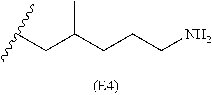 [SMILES: *CC(C)CCCN] |
| 5-4-6 | 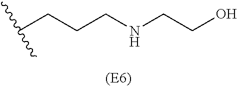 [SMILES: *CCCNCCO ; "(E6)"] |
| 5-4-7 | 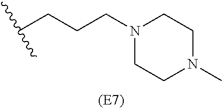 [SMILES: *CCCN1CCN(C)CC1 ; "(E7)"] |

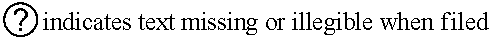

In even yet more particular embodiments, the PBAE of formula (I) is selected from the group consisting of:

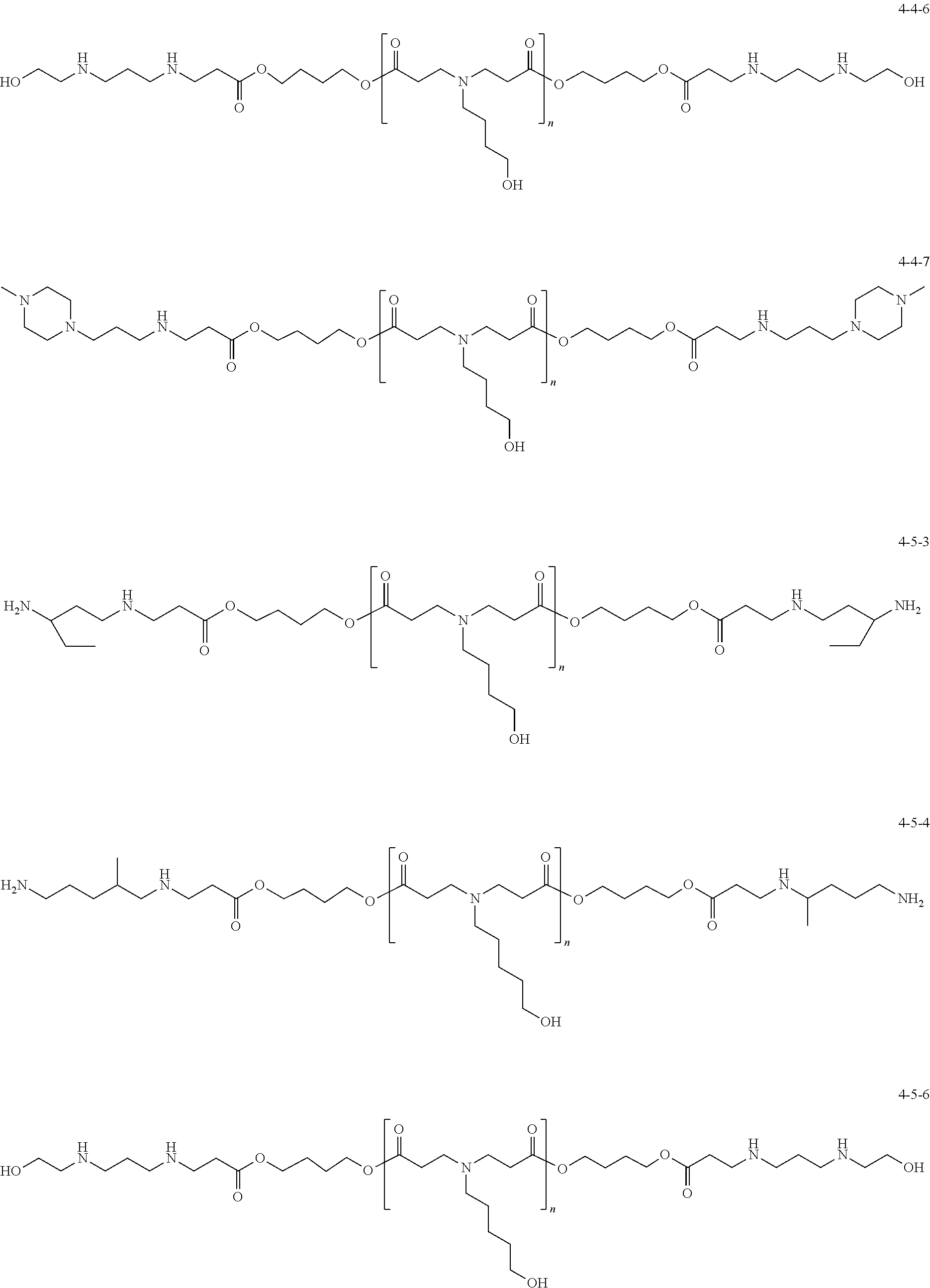

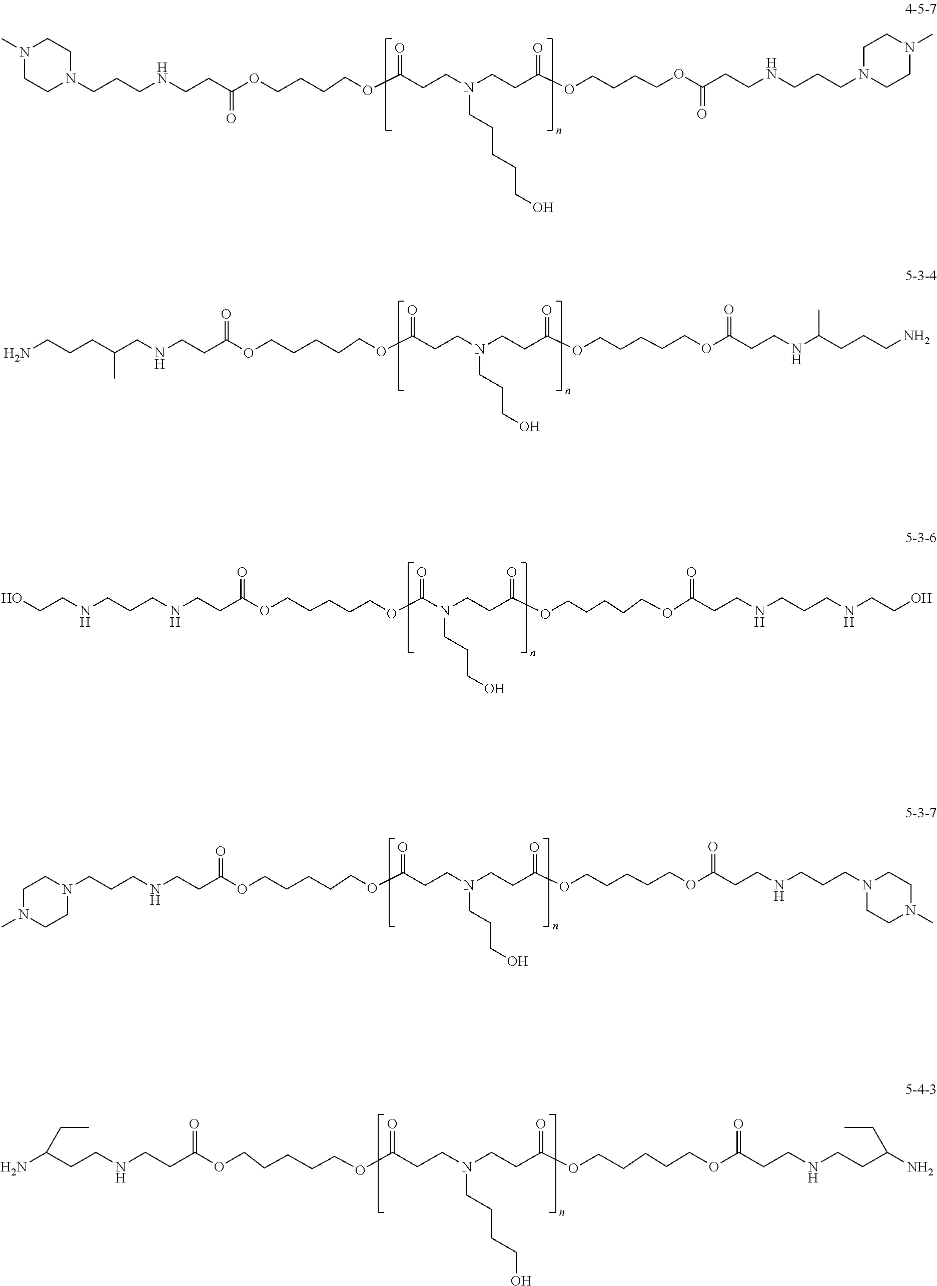

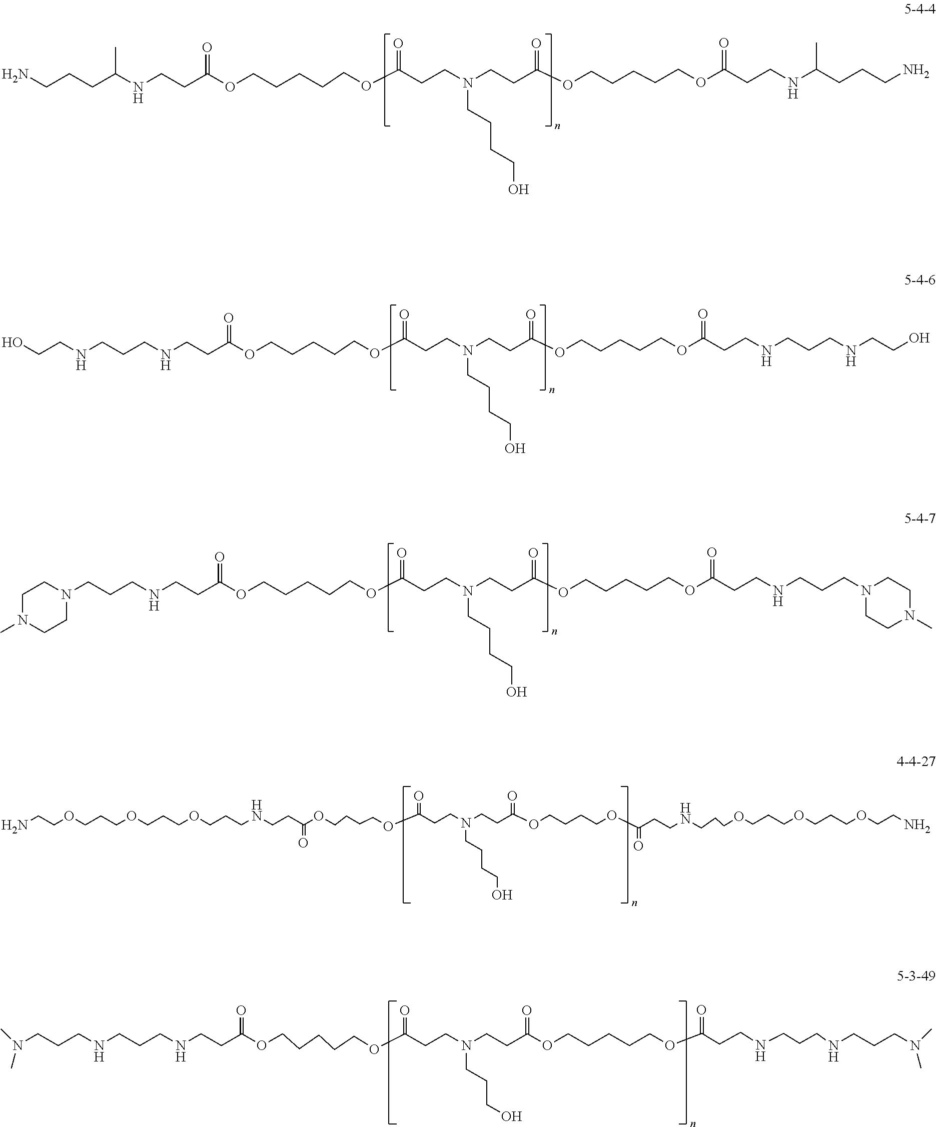

In particular embodiments, the presently disclosed subject matter provides a composition comprising a poly(beta-amino ester) (PBAE) of formula (I):

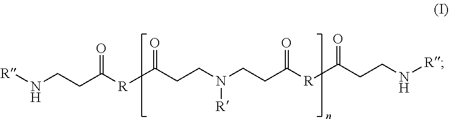

and at least one of a first genetic element that encodes a signal 2 protein, a second genetic element that encodes a signal 3 protein, and/or a third genetic element that encodes a signal 1 protein;

wherein: n is an integer from 1 to 10,000; each R is independently selected from the group consisting of:

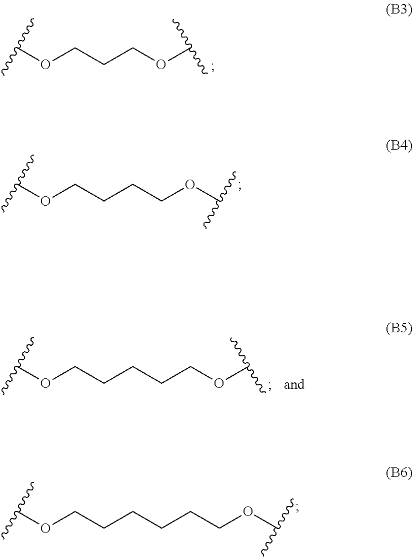 [SMILES: *OCCCO* ; *OCCCCO* ; *OCCCCCO* ; *OCCCCCCO*]

each R′ is independently selected from the group consisting of:

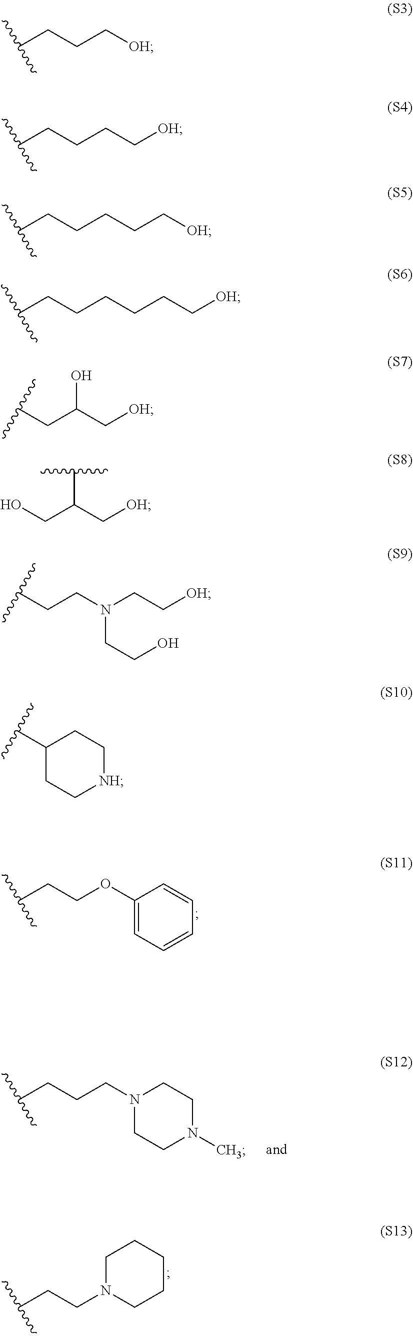

each R″ is independently selected from the group consisting of:

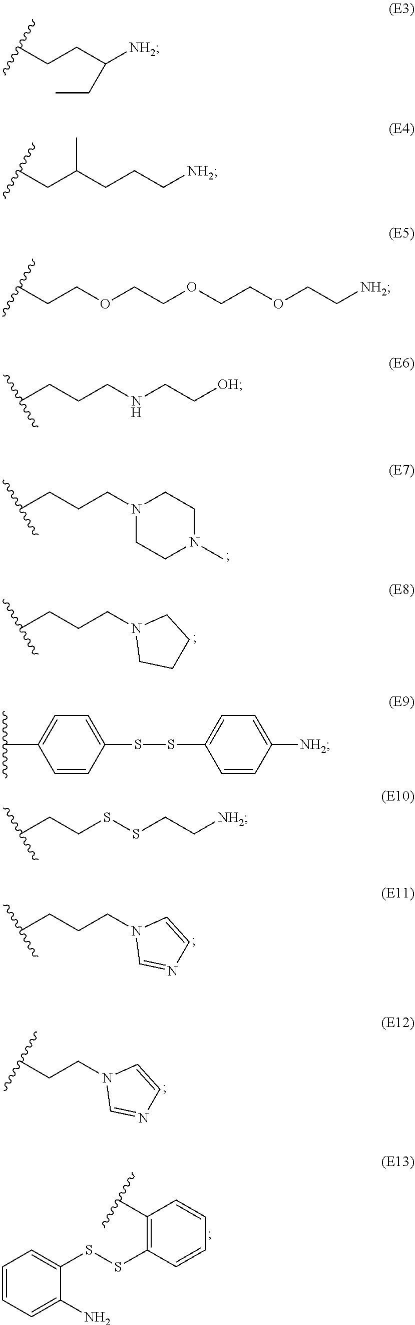

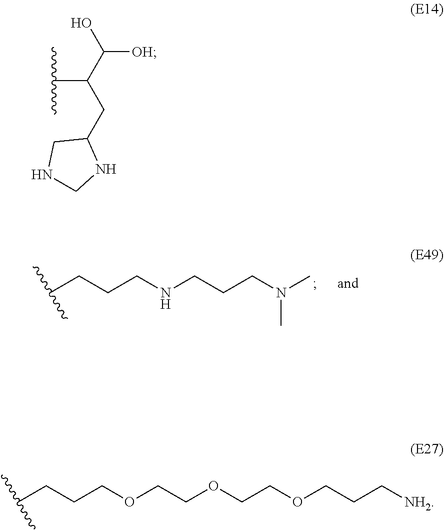 [SMILES: *C(CC1CNCN1)C(O)O ; *CCCNCCCN(C)C ; *CCCOCCOCCOCCCN]

In some embodiments, n is selected from the group consisting of an integer from 1 to 1,000; an integer from 1 to 100; an integer from 1 to 30; an integer from 5 to 20; an integer from 10 to 15; and an integer from 1 to 10.

In certain embodiments, the following poly(beta-amino ester)s were complexed with particular DNA plasmids or other nucleic acids that can overexpress signal 2 or signal 3 proteins and were used for in vitro and/or in vivo transfections of various cancer cells as indicated herein below:

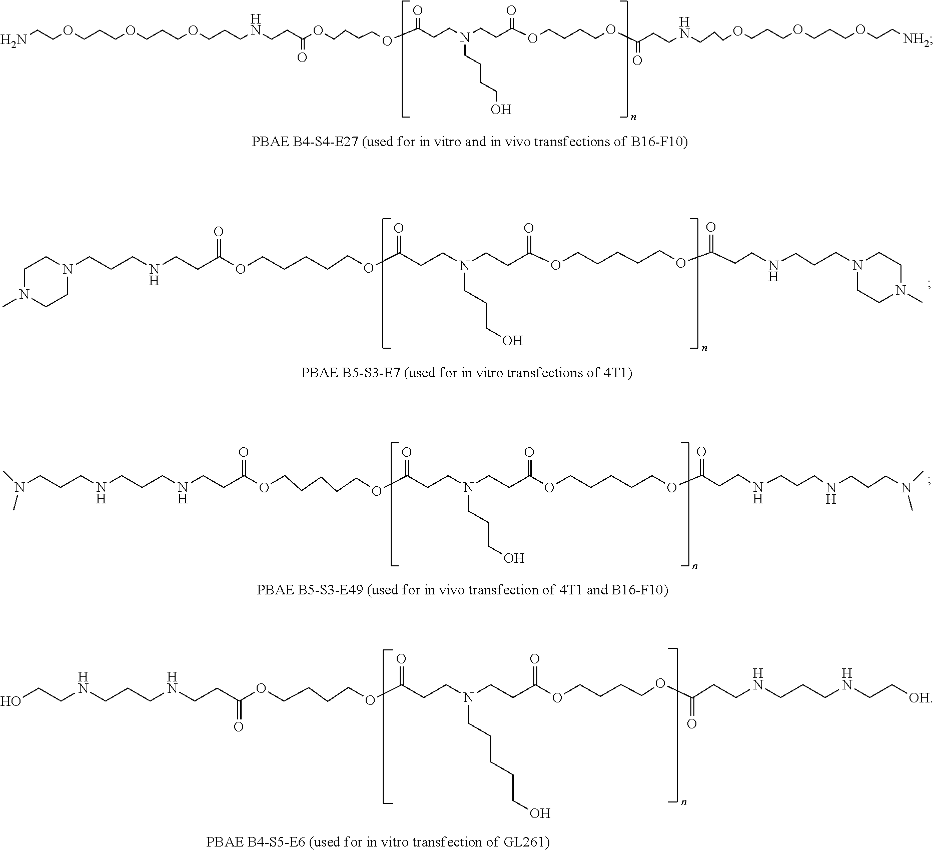

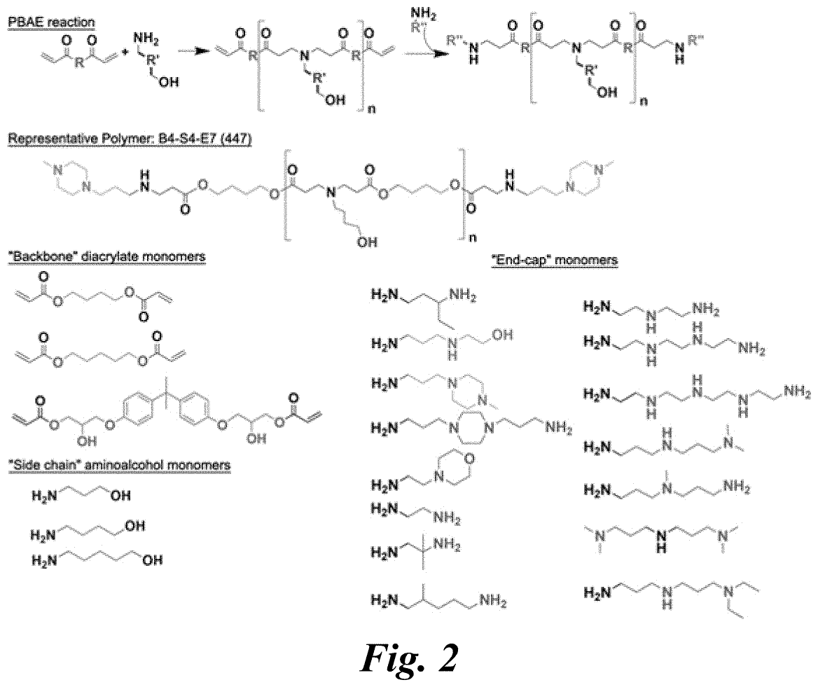

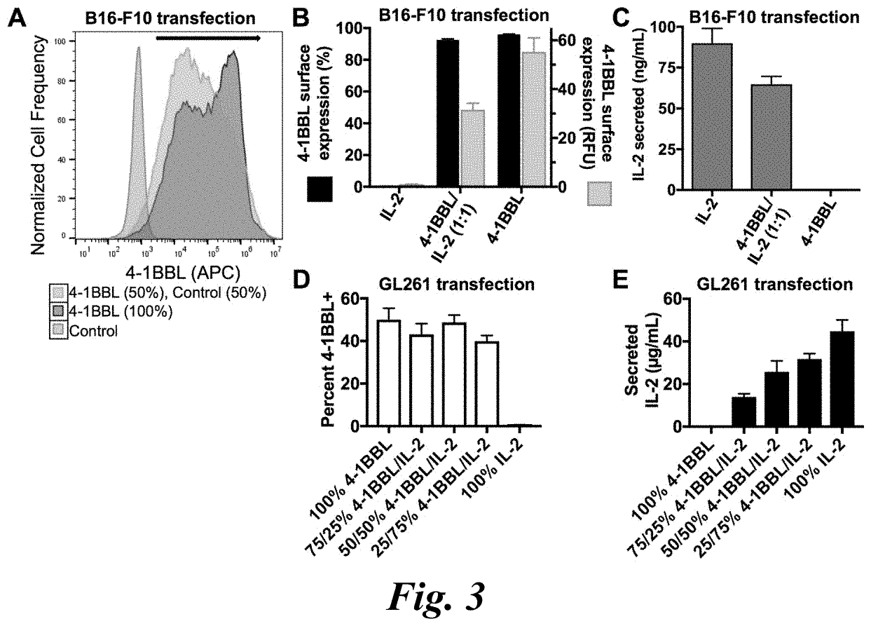

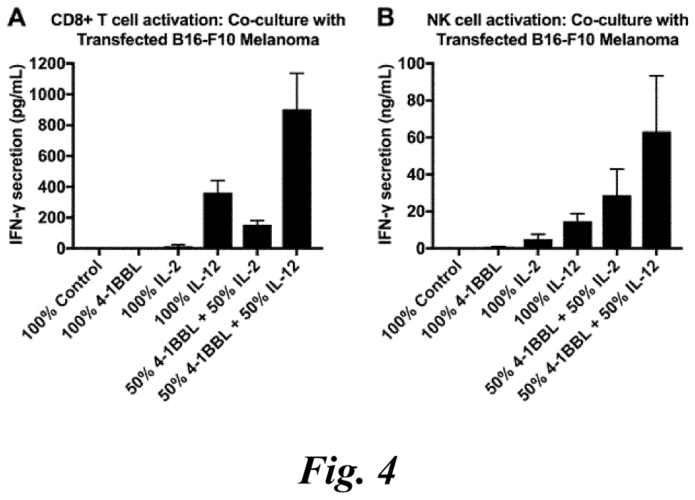

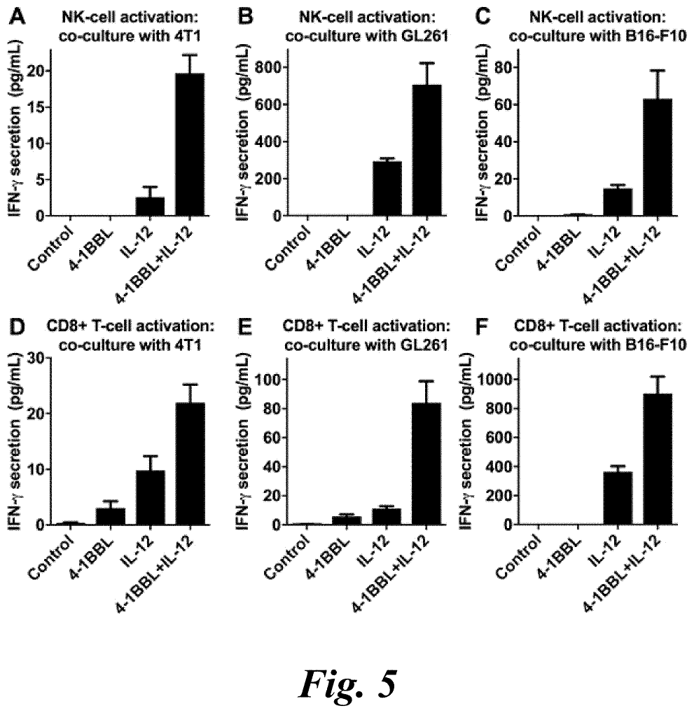

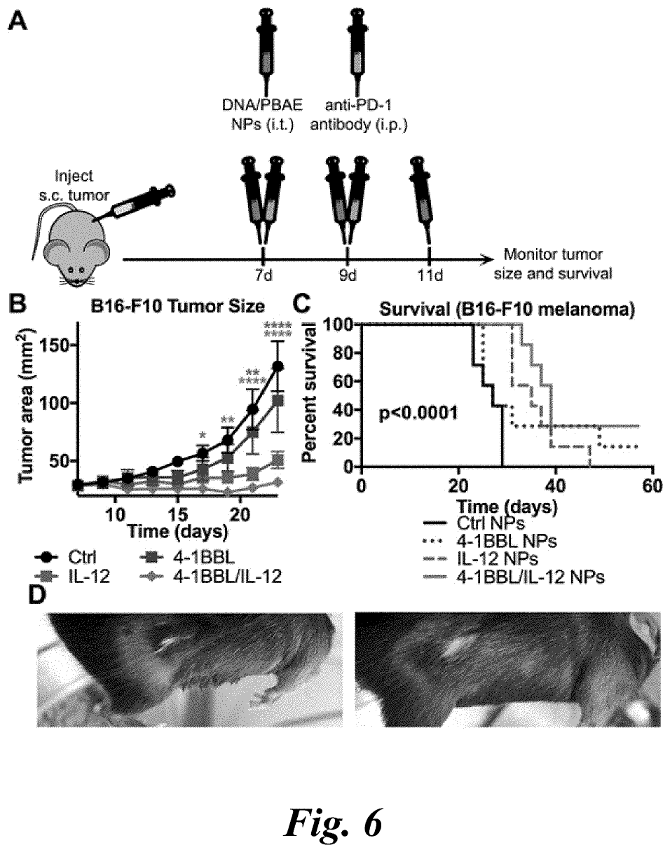

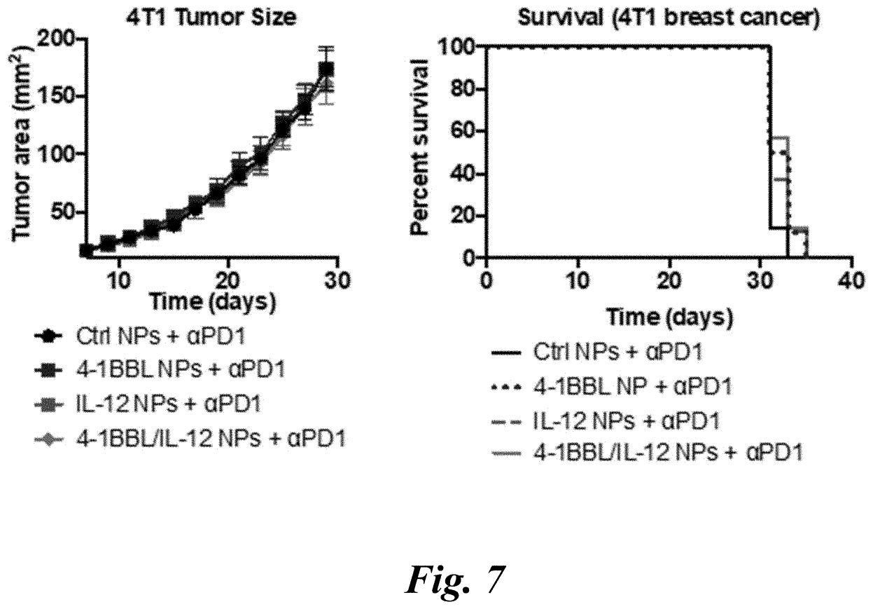

Other PBAEs may be used to further optimize transfection or for transfection of different cell and tissue types to achieve the strongest gene expression in each tumor model tested. Each PBAE is comprises one backbone monomer (“B”) polymerized with one side-chain monomer (“S”), terminated with one end-cap monomer (“E”) (FIG. 2).

In particular embodiments, the composition has a PBAE-to-DNA plasmid weight-to-weight ratio (w/w) selected from the group consisting of, in some embodiments, about 75 w/w to about 10 w/w, in some embodiments, about 50 w/w to about 20 w/w, in some embodiments, about 25 w/w, and, in some embodiments, about 50 w/w.

In certain embodiments, the linear and/or branched PBAE polymer has a molecular weight of from 5 to 10 kDa, or a molecular weight of from 10 to 15 kDa, or a molecular weight of from 15 to 25 kDa, or a molecular weight of from 25 to 50 kDa.

In certain embodiments, the presently disclosed subject matter provides a pharmaceutical formulation comprising the above-described nucleic acid molecule and a poly(beta-amino ester) (PBAE) of formula (I) in a pharmaceutically acceptable carrier.

As used herein, “pharmaceutically acceptable carrier” is intended to include, but is not limited to, water, saline, dextrose solutions, human serum albumin, liposomes, hydrogels, microparticles and nanoparticles. The use of such media and agents for pharmaceutically active compositions is well known in the art, and thus further examples and methods of incorporating each into compositions at effective levels need not be discussed here.

In yet other embodiments, the pharmaceutical formulation further comprises a nanoparticle or microparticle of the PBAE of formula (I). The PBAE polymers in some embodiments can self-assemble with nucleic acid, including plasmid DNA, to form nanoparticles which may be in the range of 50 to 500 nm in size. In embodiments, the particle has at least one dimension in the range of about 50 nm to about 500 nm, or from about 50 to about 200 nm. Exemplary particles may have an average size (e.g., average diameter) of about 50, about 75, about 100, about 125, about 150, about 200, about 250, about 300, about 400 or about 500 nm. In some embodiments, the nanoparticle has an average diameter of from about 50 nm to about 500 nm, from about 50 nm to about 300 nm, or from about 50 nm to about 200 nm, or from about 50 nm to about 150 nm, or from about 70 to 100 nm. In embodiments, the nanoparticle has an average diameter of from about 200 nm to about 500 nm. In embodiments, the nanoparticle has at least one dimension, e.g., average diameter, of about 50 to about 100 nm. Nanoparticles are usually desirable for in vivo applications. For example, a nanoparticle of less than about 200 nm will better distribute to target tissues in vivo.

In some embodiments, the presently disclosed particles may comprise other combinations of cationic polymeric blends or block co-polymers. Additional polymers include polycaprolactone (PCL), polyglycolic acid (PGA), polylactic acid (PLA), poly(acrylic acid) (PAA), poly-3-hydroxybutyrate (P3HB), poly(hydroxybutyrate-co-hydroxyvalerate), and polyethylene glycol (PEG). In embodiments, a particle includes blends of other polymer materials to modulate a particle's surface properties. For example, the blend may include non-degradable polymers that are used in the art, such as polystyrene. Thus, in embodiments, a degradable polymer or polymers from above are blended to create a copolymer system. In yet other embodiments, the presently disclosed particle comprises a polymer blend of PBAE, e.g., a mixture of PBAE polymers.

In embodiments, the particles are spherical in shape. In embodiments, the particles have a non-spherical shape. In embodiments, the particles have an ellipsoidal shape with an aspect ratio of the long axis to the short axis between 2 and 10.

In certain embodiments, nanoparticles formed through the presently disclosed procedures that encapsulate active agents, such as DNA plasmid, are themselves encapsulated into a larger nanoparticle, microparticle, or device. In some embodiments, this larger structure is degradable and in other embodiments it is not degradable and instead serves as a reservoir that can be refilled with the nanoparticles. These larger nanoparticles, microparticles, and/or devices can be constructed with any biomaterials and methods that one skilled in the art would be aware. In some embodiments they can be constructed with multi-component degradable cationic polymers as described herein. In other embodiments, they can be constructed with FDA-approved biomaterials, including, but not limited to, poly(lactic-co-glycolic acid) (PLGA). In the case of PLGA and the double emulsion fabrication process as an example, the nanoparticles are part of the aqueous phase in the primary emulsion. In the final PLGA nano- or microparticles, the nanoparticles will remain in the aqueous phase and in the pores/pockets of the PLGA nano- or microparticles. As the microparticles degrade, the nanoparticles will be released, thereby allowing sustained release of the nanoparticles comprising the active agents. In particular embodiments, the nanoparticle or microparticle of the PBAE of formula (I) is encapsulated in a poly(lactic-co-glycolic acid) (PLGA) nanoparticle or microparticle.

In some embodiments, the presently disclosed subject matter also includes a method of using and storing the polymers and particles described herein whereby a cryoprotectant (including, but not limited to, a sugar) is added to the polymer and/or particle solution and it is lyophilized and stored as a powder. Such a powder is designed to remain stable and be reconstituted easily with aqueous buffer as one skilled in the art could utilize.

In certain embodiments, the nanoparticle targeting (through biomaterial selection, nanoparticle biophysical properties, and/or a targeting ligand) is combined with transcriptional targeting of a therapeutic gene to a particular cell type (e.g., cancer cells). Transcriptional targeting includes designing nucleic acid cargo which comprises a promoter that is active in cells or tissue types of interest so that the delivered nanoparticles express the nucleic acid cargo in a tissue-specific manner.

In particular embodiments, the presently disclosed particles carry one or more of a first genetic element that encodes a signal 2 protein, a second genetic element that encodes a signal 3 protein, and/or a third genetic element that encodes a signal 1 protein. The cell may be a eukaryotic cell, such as an animal cell or plant cell. In further embodiments, the animal cell is a mammalian cell (e.g., a human cell). In some embodiments, the cell is transfected with the particles for ex vivo gene therapy. In some embodiments, the particles are delivered directly to an organism, such as mammalian subject, to thereby direct gene therapy in vivo. In particular embodiments, including for delivering nucleic acids to cells in vivo, the cell is a cancer cell or malignant cell.

For in vivo gene therapy, particles can be formulated for a variety of modes of administration, including systemic and topical or localized administration. Thus, the pharmaceutical compositions can be formulated for administration to patients by any appropriate route, including intravenous administration, intra-arterial administration, subcutaneous administration, intradermal administration, intralymphatic administration, and intra-tumoral administration. In some embodiments, the composition is lyophilized and reconstituted prior to administration.

Exemplary proteins encapsulated by the presently disclosed nanoparticles include a “Signal 1” protein, including MHC-I and MHC-II molecules, as well as a “Signal 2” protein that acts as a co-stimulatory molecule to immune cells, such as anti-CD28, 4-1BBL, CD80, CD86, and OX40L. In particular embodiments, the signal 2 protein is 4-1BBL.

More particularly, “co-stimulatory molecule,” as the term is used herein, includes a molecule on an antigen presenting cell that specifically binds a cognate co-stimulatory molecule on a T cell, thereby providing a signal which mediates a T cell response, including, but not limited to, proliferation, activation, differentiation, and the like. A co-stimulatory molecule can include, but is not limited to, anti-CD28, CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory molecule also encompasses, inter alia, an antibody that specifically binds with a co-stimulatory molecule present on a T cell, such as, but not limited to, CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

A “co-stimulatory signal”, as used herein, refers to a signal that leads to T cell proliferation and/or upregulation or downregulation of key molecules.

Exemplary Signal 3 proteins include interleukins and cytokines, such as the transforming growth factor (TGF) beta family of cytokines, including TGF-β1, TGF-β2, TGF-β3, and TGF-β4. Representative interleukins include, but are not limited to, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, and IL-36. In particular embodiments, the signal 3 protein is selected from the group consisting of IL-6, IL-7, IL-15, IL-18, IL-21, IFN-α, and IFN-β. In yet more particular embodiments, the signal 3 protein is IL-2 or IL-12.

In some embodiments, the particle further comprises a coating comprising one or more synthetic and/or natural lipids and/or lipid membranes. In particular embodiments, the at least two types of protein are attached to the coating comprising one or more synthetic and/or natural lipids and/or lipid membranes. Representative lipids suitable for use in coating the presently disclose particles include, but are not limited to, fatty acids, glycerolipids, glycerophospholipids, sphingolipids, sterol lipids, prenol lipids, saccharolipids, and polyketides.

### B. Methods of Treating a Cancer

In some embodiments, the presently disclosed subject matter provides a method for treating or diagnosing a cancer, the method comprising administering a composition or formulation comprising one or more of a first genetic element that encodes a signal 2 protein, a second genetic element that encodes a signal 3 protein, and/or a third genetic element that encodes a signal 1 protein encapsulated in a PBAE composition of formula (I) as described herein to a subject in need of treatment thereof.

More generally, the presently disclosed subject matter provides a method for reprogramming one or more cancer cells into one or more tumor-derived antigen-presenting cells (tAPCs), wherein the one or more tAPCs mimic a natural antigen-presenting cell (APC) and direct an immune response against themselves and other cancer cells, the method comprising transfecting the one or more cancer cells with composition a composition comprising at least one of a first genetic element that encodes a signal 2 protein, a second genetic element that encodes a signal 3 protein, and/or a third genetic element that encodes a signal 1 protein encapsulated in a nanoparticle comprising a cationic biomaterial or biomaterial blend.

In particular embodiments, the transfection of the one or more cancer cells promotes an immune cell activation against one or more antigens expressed on the one or more cancer cells. In more particular embodiments, the one or more tAPCs activate an antigen-specific T-cell response against MHC I+ tumor cells. In other embodiments, the one or more tAPCs provide an activating signal to one or more natural killer (NK) cells to induce anti-tumor cytotoxicity therein. In certain embodiments, the one or more tAPCs activate an antigen-independent NK cell response against MHC I−/low tumor cells. Importantly, the presently disclosed methods can induce a systemic immune response resulting in cell death of distant metastases.

Such methods can be used to treat cancer, the method comprising transfecting one or more cancer cells in a subject in need of treatment thereof a composition disclosed herein.

Any cancer may be treated using the methods described herein. A “cancer” in a subject refers to the presence of cells possessing characteristics typical of cancer-causing cells, for example, uncontrolled proliferation, loss of specialized functions, immortality, significant metastatic potential, significant increase in anti-apoptotic activity, rapid growth and proliferation rate, and certain characteristic morphology and cellular markers. In some circumstances, cancer cells will be in the form of a tumor; such cells may exist locally within a subject, or circulate in the blood stream as independent cells, for example, leukemic cells.

A cancer can include, but is not limited to, acute lymphocytic leukemia, acute myelogenous leukemia, angiosarcoma, basal cell carcinoma, bladder cancer, brain cancer (e.g., gliomas), breast cancer, cervical cancer, choriocarcinoma, colon cancer, colorectal cancer, corpus uteri cancer, endocrine cancer, esophageal cancer, Ewing's Sarcoma, eye or ocular cancer, gastrointestinal cancer, head cancer, head and neck cancer, hemangioendothelioma, hemangiomas, hepatocellular carcinoma (HCC), Kaposi's Sarcoma, larynx cancer, leukemia/lymphoma, liver cancer, lung cancer, lymphoma, lymphangiogenesis, melanoma, mouth/pharynx cancer, neck cancer, neuroblastoma, neurofibromatosis, oral cancer, ovarian cancer, pancreatic cancer, prostate cancer, rectal cancer, renal cancer, rhabdomyosarcoma, stomach cancer, skin cancer, small cell lung cancer, squamous cell carcinoma, testicular cancer, throat cancer, tuberous sclerosis, urinary cancer, uterine cancer, Wilms Tumor, benign and malignant tumors, and adenomas.

In particular embodiments, the cancer is selected from the group consisting of a melanoma, a breast cancer, including triple-negative breast cancer, colorectal cancer, liver cancer, and brain cancer, including gliomas.

In certain embodiments, the presently disclosed method further comprises administering to the subject one or more therapeutic agents simultaneously or sequentially with the PBAE composition of formula (I) or a formulation thereof.

As used herein, the term “treating” can include reversing, alleviating, inhibiting the progression of, preventing or reducing the likelihood of the disease, disorder, or condition to which such term applies, or one or more symptoms or manifestations of such disease, disorder or condition. Preventing refers to causing a disease, disorder, condition, or symptom or manifestation of such, or worsening of the severity of such, not to occur. Accordingly, the presently disclosed compounds can be administered prophylactically to prevent or reduce the incidence or recurrence of the disease, disorder, or condition.

As used herein, the term “inhibit,” and grammatical derivations thereof, refers to the ability of a presently disclosed compound, e.g., a presently disclosed composition of formula (I), to block, partially block, interfere, decrease, or reduce the growth and/or metastasis of a cancer cell. Thus, one of ordinary skill in the art would appreciate that the term “inhibit” encompasses a complete and/or partial decrease in the growth and/or metastasis of a cancer cell, e.g., a decrease by at least 10%, in some embodiments, a decrease by at least 20%, 30%, 50%, 75%, 95%, 98%, and up to and including 100%.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the makeup of the pharmaceutical composition, the target tissue, and the like.

The term “combination” is used in its broadest sense and means that a subject is administered at least two agents, more particularly a composition of formula (I) and at least one therapeutic agent. More particularly, the term “in combination” refers to the concomitant administration of two (or more) active agents for the treatment of a, e.g., single disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one but not the other). The single dosage form may include additional active agents for the treatment of the disease state.

Further, the compounds of formula (I) described herein can be administered alone or in combination with adjuvants that enhance stability of the compounds of formula (I), alone or in combination with one or more therapeutic agents, facilitate administration of pharmaceutical compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of a composition of formula (I) and at least one additional therapeutic agent can be varied so long as the beneficial effects of the combination of these agents are achieved. Accordingly, the phrase “in combination with” refers to the administration of a composition of formula (I) and at least one additional therapeutic agent either simultaneously, sequentially, or a combination thereof. Therefore, a subject administered a combination of a composition of formula (I) and at least one additional therapeutic agent can receive composition of formula (I) and at least one additional therapeutic agent at the same time (i.e., simultaneously) or at different times (i.e., sequentially, in either order, on the same day or on different days), so long as the effect of the combination of both agents is achieved in the subject.

When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the composition of formula (I) and at least one additional therapeutic agent are administered simultaneously, they can be administered to the subject as separate pharmaceutical compositions, each comprising either a composition of formula (I) or at least one additional therapeutic agent, or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of a composition of formula (I) and at least one additional therapeutic agent is greater than the sum of the biological activities of the respective agents when administered individually.

Synergy can be expressed in terms of a “Synergy Index (SI),” which generally can be determined by the method described by F. C. Kull et al., Applied Microbiology 9, 538 (1961), from the ratio determined by:

Qₐ/Q_{A}+Q_{b}/Q_{B}=Synergy Index (SI)

wherein:

Q_{A} is the concentration of a component A, acting alone, which produced an end point in relation to component A;

Qₐ is the concentration of component A, in a mixture, which produced an end point;

Q_{B} is the concentration of a component B, acting alone, which produced an end point in relation to component B; and

Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

In the various embodiments described above, the presently disclosed nanoparticles can be administered in a variety of forms depending on the desired route and/or dose. The presently disclosed nanoparticles can be administered in a pharmaceutically acceptable carrier. As used herein, “pharmaceutically acceptable carrier” is intended to include, but is not limited to, water, saline, dextrose solutions, human serum albumin, liposomes, hydrogels, microparticles and nanoparticles.

Depending on the specific conditions being treated, the presently disclosed nanoparticles may be formulated into liquid or solid dosage forms and administered systemically or locally. The agents may be delivered, for example, in a timed- or sustained-low release form as is known to those skilled in the art. Techniques for formulation and administration may be found in Remington: The Science and Practice of Pharmacy (20th ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articular, intra-sternal, intra-synovial, intra-hepatic, intralesional, intracranial, intraperitoneal, intranasal, or intraocular injections or other modes of delivery.

While the form and/or route of administration can vary, in some embodiments the presently disclosed nanoparticles or pharmaceutical composition is administered parenterally (e.g., by subcutaneous, intravenous, or intramuscular administration), or in some embodiments is administered directly to the lungs. Local administration to the lungs can be achieved using a variety of formulation strategies including pharmaceutical aerosols, which may be solution aerosols or powder aerosols. Powder formulations typically comprise small particles. Suitable particles can be prepared using any means known in the art, for example, by grinding in an airjet mill, ball mill or vibrator mill, sieving, microprecipitation, spray-drying, lyophilization or controlled crystallization. Typically, particles will be about 10 microns or less in diameter. Powder formulations may optionally contain at least one particulate pharmaceutically acceptable carrier known to those of skill in the art. Examples of suitable pharmaceutical carriers include, but are not limited to, saccharides, including monosaccharides, disaccharides, polysaccharides and sugar alcohols such as arabinose, glucose, fructose, ribose, mannose, sucrose, trehalose, lactose, maltose, starches, dextran, mannitol or sorbitol. Alternatively, solution aerosols may be prepared using any means known to those of skill in the art, for example, an aerosol vial provided with a valve adapted to deliver a metered dose of the composition. Where the inhalable form of the active ingredient is a nebulizable aqueous, organic or aqueous/organic dispersion, the inhalation device may be a nebulizer, for example a conventional pneumatic nebulizer such as an airjet nebulizer, or an ultrasonic nebulizer, which may contain, for example, from 1 to 50 mL, commonly 1 to 10 mL, of the dispersion; or a hand-held nebulizer which allows smaller nebulized volumes, e.g. 10 μL to 100 μL.

For injection, the agents of the disclosure may be formulated and diluted in aqueous solutions, such as in physiologically compatible buffers such as Hank's solution, Ringer's solution, or physiological saline buffer.

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, the compositions of the present disclosure, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions and the like, for oral ingestion by a subject (e.g., patient) to be treated.

For nasal or inhalation delivery, the agents of the disclosure also may be formulated by methods known to those of skill in the art, and may include, for example, but not limited to, examples of solubilizing, diluting, or dispersing substances such as, saline, preservatives, such as benzyl alcohol, absorption promoters, and fluorocarbons.

### C. Kits

In some embodiments, the presently disclosed subject matter provides a kit. In general, the presently disclosed kit contains some or all of the components, reagents, supplies, and the like to practice a method according to the presently disclosed subject matter. In general, a presently disclosed kit contains some or all of the components, reagents, supplies, and the like to practice a method according to the presently disclosed subject matter. In some embodiments, the term “kit” refers to any intended article of manufacture (e.g., a package or a container) comprising a presently disclosed biodegradable particle formulation. In some embodiments, the kit can be packaged in a divided or undivided container, such as a carton, bottle, ampule, tube, and the like. The presently disclosed compositions can be packaged in dried, lyophilized, or liquid form. Additional components provided can include vehicles for reconstitution of dried components. Preferably all such vehicles are sterile and apyrogenic so that they are suitable for injection into a patient without causing adverse reactions. In certain embodiments, the kit further comprises one of more of multiple dosage units of the composition, a pharmaceutically acceptable carrier, a device for administration of the composition, instructions for use, and combinations thereof.

### D. Definitions

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

As used herein the term “monomer” refers to a molecule that can undergo polymerization, thereby contributing constitutional units to the essential structure of a macromolecule or polymer.

A “polymer” is a molecule of high relative molecule mass, the structure of which essentially comprises the multiple repetition of unit derived from molecules of low relative molecular mass, i.e., a monomer.

As used herein, an “oligomer” includes a few monomer units, for example, in contrast to a polymer that potentially can comprise an unlimited number of monomers. Dimers, trimers, and tetramers are non-limiting examples of oligomers.

Further, as used herein, the term “nanoparticle,” refers to a particle having at least one dimension in the range of about 1 nm to about 1000 nm, including any integer value between 1 nm and 1000 nm (including about 1, 2, 5, 10, 20, 50, 60, 70, 80, 90, 100, 200, 500, and 1000 nm and all integers and fractional integers in between). In some embodiments, the nanoparticle has at least one dimension, e.g., a diameter, of about 100 nm. In some embodiments, the nanoparticle has a diameter of about 200 nm. In other embodiments, the nanoparticle has a diameter of about 500 nm. In yet other embodiments, the nanoparticle has a diameter of about 1000 nm (1 μm). In such embodiments, the particle also can be referred to as a “microparticle. Thus, the term “microparticle” includes particles having at least one dimension in the range of about one micrometer (μm), i.e., 1×10⁻⁶ meters, to about 1000 The term “particle” as used herein is meant to include nanoparticles and microparticles.

It will be appreciated by one of ordinary skill in the art that nanoparticles suitable for use with the presently disclosed methods can exist in a variety of shapes, including, but not limited to, spheroids, rods, disks, pyramids, cubes, cylinders, nanohelixes, nanosprings, nanorings, rod-shaped nanoparticles, arrow-shaped nanoparticles, teardrop-shaped nanoparticles, tetrapod-shaped nanoparticles, prism-shaped nanoparticles, and a plurality of other geometric and non-geometric shapes. In particular embodiments, the presently disclosed nanoparticles have a spherical shape.

“Associated with”: When two entities are “associated with” one another as described herein, they are linked by a direct or indirect covalent or non-covalent interaction. Preferably, the association is covalent. Desirable non-covalent interactions include hydrogen bonding, van der Waals interactions, hydrophobic interactions, magnetic interactions, electrostatic interactions, and the like.

“Biocompatible”: The term “biocompatible”, as used herein is intended to describe compounds that are not toxic to cells. Compounds are “biocompatible” if their addition to cells in vitro results in less than or equal to 20% cell death, and their administration in vivo does not induce inflammation or other such adverse effects.

“Biodegradable”: As used herein, “biodegradable” compounds are those that, when introduced into cells, are broken down by the cellular machinery or by hydrolysis into components that the cells can either reuse or dispose of without significant toxic effect on the cells (i.e., fewer than about 20% of the cells are killed when the components are added to cells in vitro). The components preferably do not induce inflammation or other adverse effects in vivo. In certain preferred embodiments, the chemical reactions relied upon to break down the biodegradable compounds are uncatalyzed.

“Peptide” or “protein”: A “peptide” or “protein” comprises a string of at least three amino acids linked together by peptide bonds. The terms “protein” and “peptide” may be used interchangeably. Peptide may refer to an individual peptide or a collection of peptides. Inventive peptides preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in an inventive peptide may be modified, for example, by the addition of a chemical entity such as a carbohydrate group, a phosphate group, a farnesyl group, an isofarnesyl group, a fatty acid group, a linker for conjugation, functionalization, or other modification, etc. In a preferred embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide.

“Polynucleotide” or “oligonucleotide”: Polynucleotide or oligonucleotide refers to a polymer of nucleotides. Typically, a polynucleotide comprises at least three nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, 0(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages).

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration and are not to be construed as limiting in any manner to make compounds of the disclosure by other methods.

### Example 1

### Non-Viral Genetic Reprogramming of Tumor Cells for Self-Directed Cancer Immunotherapy

### 1.1 Background

Advances in cancer immunotherapy have great potential for combatting tumors that are refractory to conventional treatments. Redman et al., 2016. T-cells can be primed to kill cancer cells by antigen-presenting cells (APCs), which present three crucial signals: signal 1, major histocompatibility complex (WIC) I with a tumor antigen (Ag) peptide; signal 2, a co-stimulatory molecule; and signal 3, secreted cytokines that promote T-cell recruitment, growth, and differentiation. Ben-Akiva et al, 2017; Curtsinger et al., 1999.

The presently disclosed subject matter uses synthetic nanoparticles (NPs) to transfect cancer cells, e.g., melanoma cells, with DNA encoding a signal 2 co-stimulatory molecule and signal 3 cytokine, effectively reprogramming these cells into tumor-derived APCs (tAPCs). In vitro assays show T-cell stimulation by melanoma tAPCs and intratumoral injection of NPs into a murine B16-F10 melanoma shows significantly slowed tumor growth. This reprogramming approach represents a novel strategy for immunotherapy that could have potentially broad impact on many types of hard-to-treat cancer.

### 1.2 Materials and Methods

A poly(beta-amino ester) (PBAE) for non-viral gene delivery was synthesized by Michael addition and used to transfect B16-F10 melanoma cells in vitro as previously described, using red fluorescent protein (RFP) to optimize NP formulation. Tzeng et al., 2013. Cells were then transfected with 4-1BBL (signal 2) and IL-2 (signal 3) plasmids and co-cultured with antigen-specific CD8+ T-cells in vitro. T-cell activation was assessed by IFN-γ secretion. For in vivo efficacy, C57BL/6 mice with subcutaneous B16-F10 tumors were injected once intratumorally (i.t.) with PBAE/DNA NPs encoding either 4-1BBL or a control gene. Tumor size was assessed over time using calipers.

### 1.3 Results and Discussion

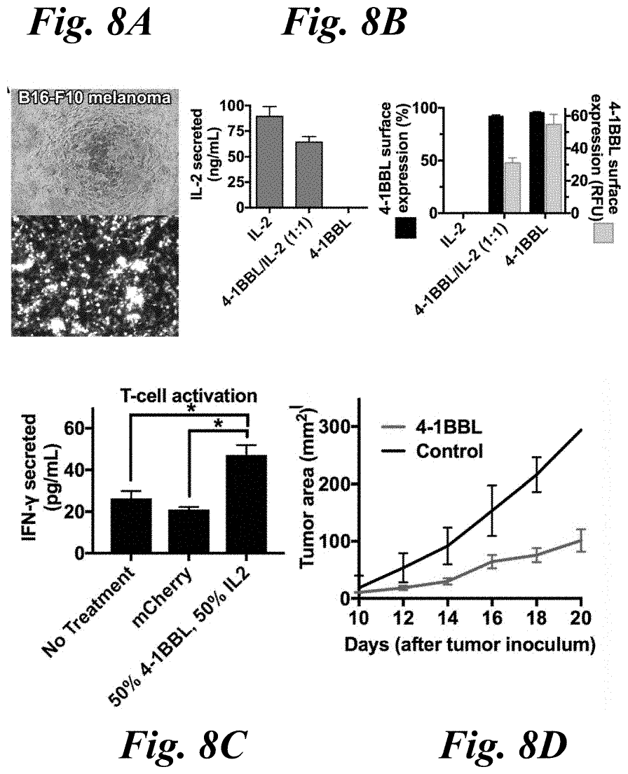

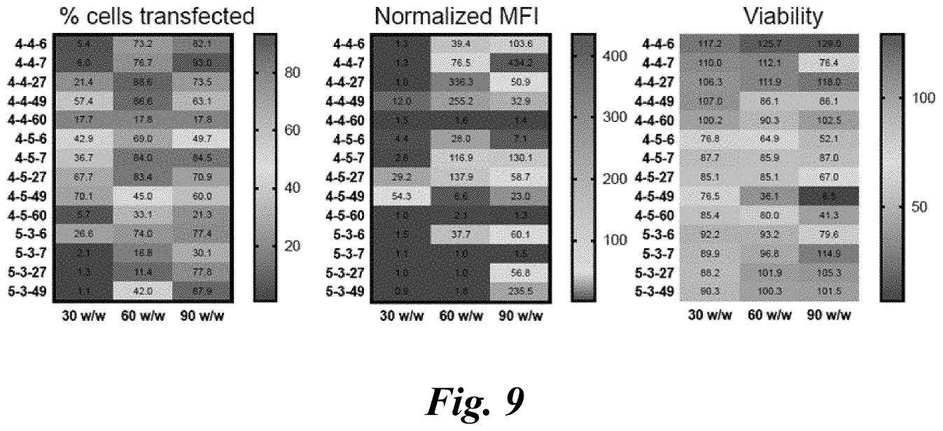

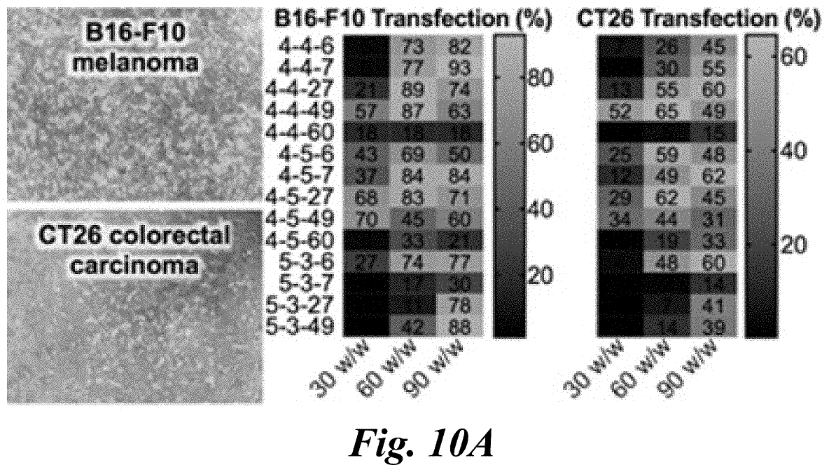

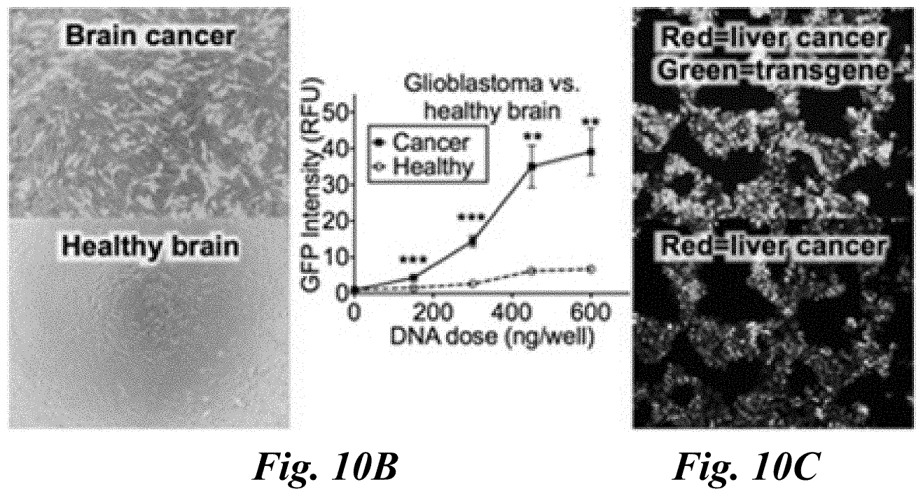

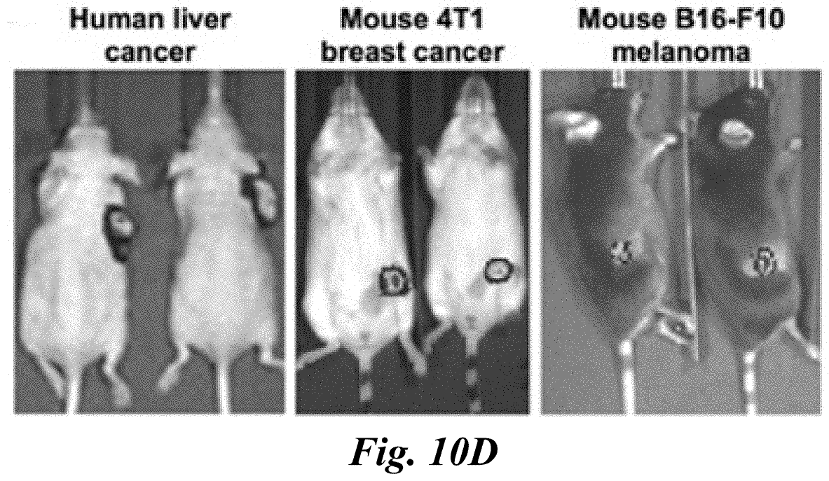

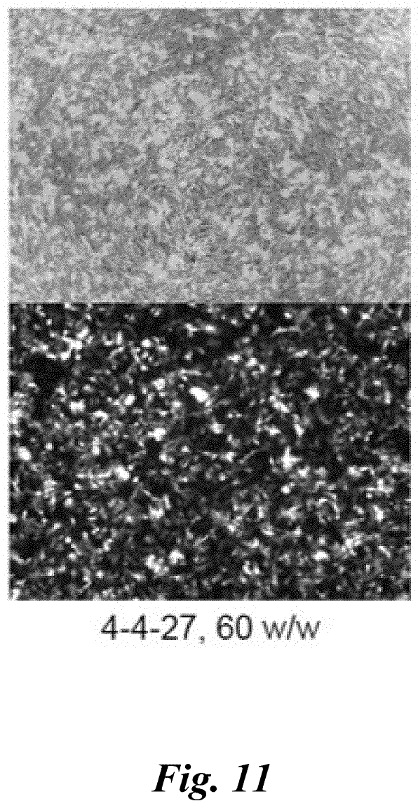

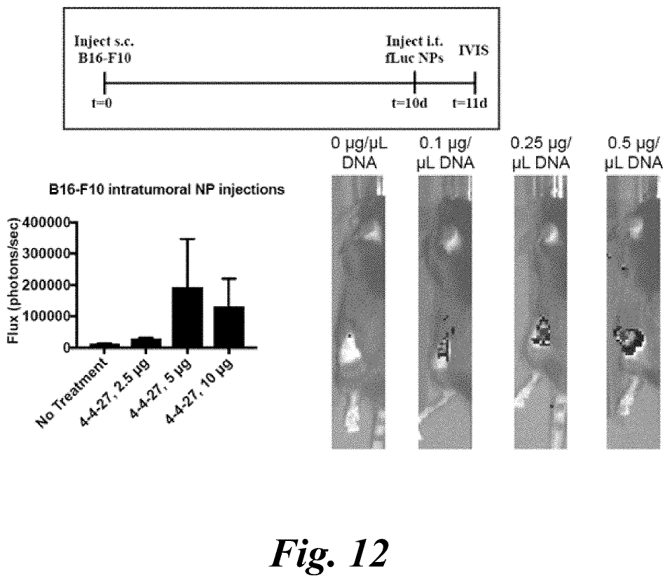

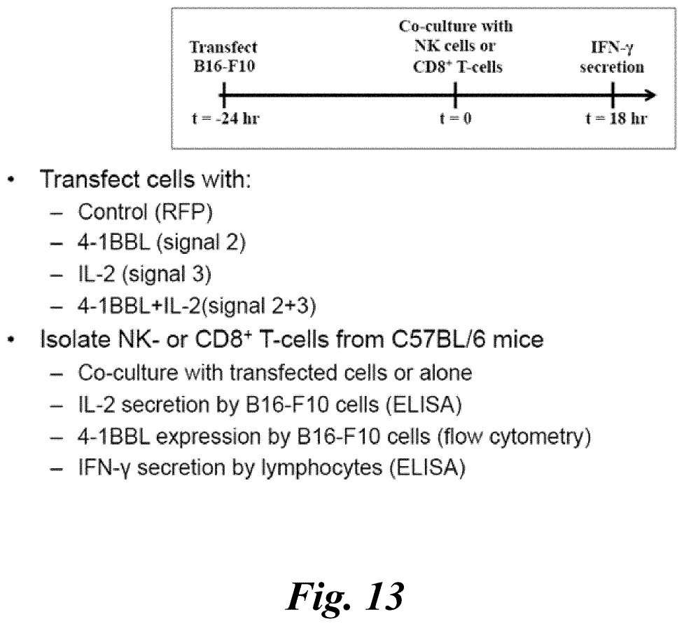

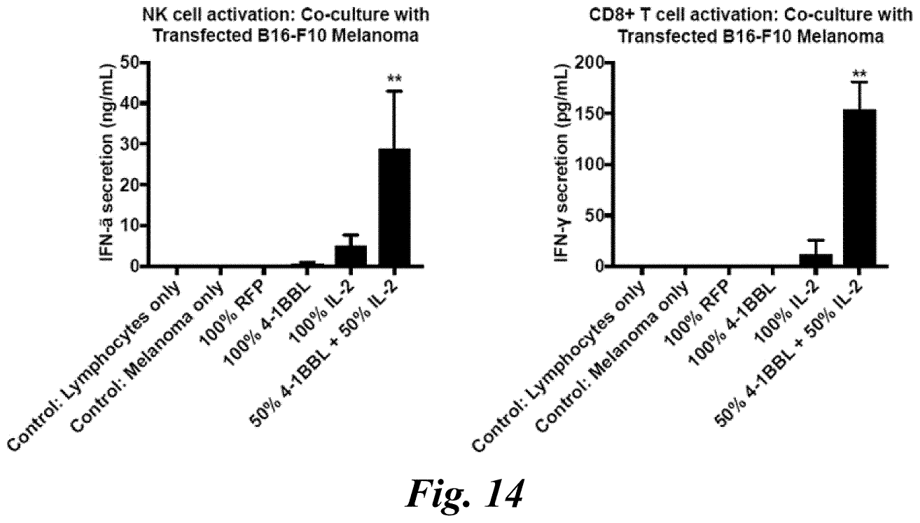

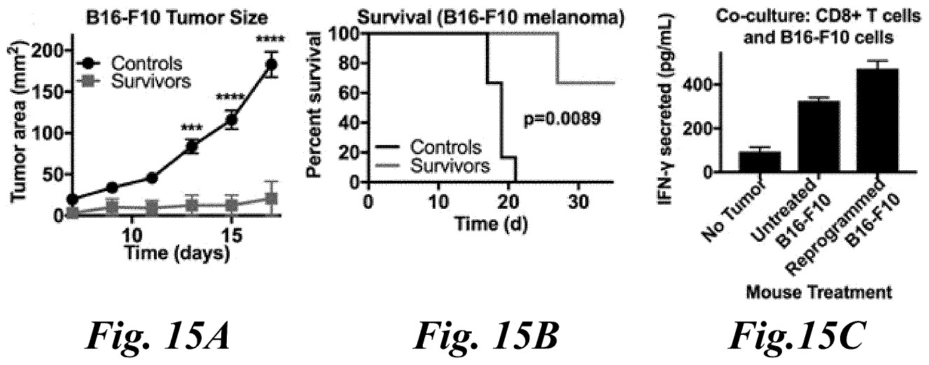

B16-F10 melanoma cells can be transfected with high (>90%) efficacy using PBAEs (FIG. 8A). Cells also can be co-transfected with signal 2 (4-1BBL) and signal 3 (IL-2). Surface 4-1BBL expression was measured by flow cytometry and secreted IL-2 was measured by ELISA (FIG. 1B). The relative expression of each can be tailored by the ratio of plasmids used in transfection. Transfected B16-F10 tAPCs induced significantly greater (p<0.05) IFN-γ secretion by CD8+ T-cells after co-culture, indicating their ability to activate T-cells (FIG. 1C). Crucially, injection of PBAE/DNA NPs into a tumor resulted in substantially slowed tumor growth (FIG. 1D) in an aggressive murine melanoma model that is generally known to have low immunogenicity.

### 1.4 Summary

PBAE NPs can transfect tumor cells with co-stimulatory molecules and immunostimulatory cytokines to reprogram them, leading to decreased tumor growth. Importantly, by hijacking the intrinsic expression of signal 1 by tumor cells, this acellular, off-the-shelf immunotherapy is antigen-agnostic and has the potential to be broadly applicable to multiple types of hard-to-treat tumors across patients.

### Example 2

### In Situ Genetic Engineering of Tumors for Long-Lasting and Systemic Immunotherapy

### 2.1 Overview

Cancer immunotherapy has been the subject of extensive research, but highly effective and broadly applicable methods remain elusive. Moreover, a general approach to engender endogenous patient-specific cellular therapy, without the need for a priori knowledge of tumor antigen, ex vivo cellular manipulation, or cellular manufacture, could dramatically reduce costs and broaden accessibility. The presently disclosed subject matter, in some embodiments, provides a biotechnology based on synthetic, biodegradable nanoparticles that can genetically reprogram cancer cells and their microenvironment in situ so that the cancer cells can act as tumor-associated antigen-presenting cells (tAPCs) by inducing co-expression of a costimulatory molecule (4-1BBL) and immunostimulatory cytokine (IL-12). In B16-F10 melanoma and MC38 colorectal carcinoma mouse models, reprogramming nanoparticles in combination with checkpoint blockade significantly reduced tumor growth over time and, in some cases, cleared the tumor, leading to long-term survivors that were then resistant to the formation of new tumors upon rechallenge at a distant site. In vitro and in vivo analyses confirmed that locally delivered tAPC-reprogramming nanoparticles led to a significant cell-mediated cytotoxic immune response with systemic effects. The systemic tumor-specific and cell-mediated immunotherapy response was achieved without requiring a priori knowledge of tumor-expressed antigens and reflects the translational potential of this nanomedicine.

There is an urgent need for improved cancer immunotherapies. The nanoparticles described here deliver genes to stimulate the immune system to specifically kill tumor cells. This synthetic, biodegradable system avoids the use of common gene delivery materials, such as viruses, which can have safety concerns and manufacturing limitations. Local nanoparticle delivery evades adverse side effects stemming from systemic administration of immune-activating therapeutics. Importantly, the presently disclosed technology causes a tumor-targeting response but does not require prior knowledge of a particular patient's gene expression profile; thus, it can serve as a platform to combat many different solid cancers. Moreover, local nanoparticle administration causes a systemic cellular immune response, which has the potential to lead to better outcomes in the context of recurrence or metastasis.

### 2.2 Background

Immunotherapy has been used successfully in the clinic to treat certain cancers that do not respond to conventional treatment. Redman et al., 2016. A critical goal of immunotherapy is the activation of a cell-mediated immune response that can specifically kill tumor cells. Mellman et al., 2011. Under ideal circumstances, a cytotoxic antitumor response could be generated via coordinated signaling between antigen-presenting cells (APCs) and CD8+ T cells. Signals important for T cell activation include signal 1, the tumor antigen in the context of major histocompatibility complex (MHC) class I; signal 2, surface-bound costimulatory molecules, Ben-Akiva et al., 2017; and signal 3, secreted immunostimulatory cytokines that contribute to cell recruitment and differentiation. Curtsinger et al., 1999.

Engineering of a patient's natural APCs to enhance this interaction often is constrained by high costs and safety risks of ex vivo cell manipulation, Banchereua and Palucka, 2005; Kantoff et al., 2010; and Anguille et al., 2014, or the technical challenges of targeted in situ APC manipulation. Anguille et al., 2014; Tacken et al., 2007. The use of artificial APCs (aAPCs), Wang et al., 2017; Eggermont et al., 2014, generally composed of biomimetic synthetic particles, often still requires ex vivo cell manipulation, Kantoff et al., 2010, and the production of tumor- and patient-specific antigen:MHC complexes for aAPC manufacturing is inefficient. Further, the best antigens to use in a given setting are unclear, vary between patients, and require a priori knowledge before treatment, and tumor neoantigen identification remains a major challenge in the field, as well as being limited in its applicability to different patients. Wang and Wang, 2017. Additionally, cancer cells avoid immune surveillance using several strategies, such as the often unpredictable variability in tumor antigen expression, as well as the expression of immunosuppressive signals by tumor cells. The heterogeneous tumor environment therefore limits the efficacy of targeting single tumor-associated antigens via aAPCs or delivery of specific tumor antigens as vaccines. Shi et al., 2017; Chen and Mellman, 2013.

### 2.3 Scope of Study

The presently disclosed subject matter, in some embodiments, provides an in situ vaccination strategy that takes advantage of the intrinsic expression of signal 1 (antigen:MHC) by many tumor cells, Comber and Philip, 2014, allowing the technology itself to remain antigen-agnostic, not requiring a priori knowledge of potential neoantigens. Tumor cells are engineered directly in vivo by safe synthetic, biodegradable gene-delivery nanoparticles composed of poly(beta-amino ester)s (PBAEs), which induce simultaneous expression of the costimulatory molecule 4-1BBL (signal 2), Zhang et al., 2007; Chacon et al., 2013, and the secreted cytokine IL-12 (signal 3). Xu et al., 2010; Ni et al., 2012. 4-1BBL has been shown to bias the immune system toward a CD8+ T cell-driven cytotoxic response, Zhang et al., 2007; Chacon et al., 2013, and to stimulate other components of the immune system, including natural killer (NK) cells and APCs. Bartkowiak and Curran, 2015. IL-12 also is known to promote NK cell activity, Ni et al., 2012; Hsu et al., 2018, which is particularly important in the case of tumor cells that downregulate MHC I expression to avoid immune surveillance. The resulting co-expression of signals 1, 2, and 3 reprograms tumor cells and their microenvironment into what is termed “tumor-associated APCs” (tAPCs).

The delivery or expression of soluble cytokines, Emtage et al., 1999; Narvaiza et al., 2000; Nomura et al., 2001, or adjuvants, Fan et al., 2017; Hanson et al., 2015, either systemically or locally, has been reported, as has the delivery of immunostimulatory agents to elicit responses from natural professional APCs. Fan et al., 2017; Cheng et al., 2018; and Zhu et al., 2017. These studies highlight the potential of engineering the microenvironment with biological molecules to enhance cellular immune responses.

In the presently disclosed approach, however, a biodegradable non-viral nanoparticle that induces the overexpression of both signals 2 and 3 on signal 1-bearing tumor cells is delivered. This approach directly activates T cells in the context of the tumor antigen, leading to an antigen-specific cellular response despite the antigen-free technology. Local expression of these immune-stimulatory molecules is crucial: Systemic delivery of signal 2 agonists and cytokines can cause adverse side effects, Lasek et al., 2014; Di Giacomo et al., 2010; and Leonard et al., 1997, while the improved function of CAR-T cells expressing signal 2 underscores the importance of co-stimulation as a part of immunotherapies. Cheng et al., 2018. Local gene delivery to overexpress cytokines and costimulatory signal 2 in the tumor itself is therefore a promising strategy to address this issue.

Further, while conventional virus-based delivery can be effective for in vivo gene transfer, Guenther et al., 2014, here, biodegradable PBAE-based nanoparticles (NPs) are used, utilizing a non-viral biotechnology to deliver DNA to cancer cells with high efficacy and specificity over healthy tissue. Tzeng et al., 2013; Guerrero-Cazares et al., 2014. Notably, this approach avoids the intrinsic immunogenicity or toxicity of more traditional gene transfer vectors, such as viruses and lipid nanoparticles, Xue et al., 2014; Vangasseri et al., 2006, while also facilitating large and flexible DNA cargo carrying capacity.

Here, the tAPC reprogramming strategy is tested in a B16-F10 murine model of melanoma, which is challenging to treat by immunotherapy. The strong effect of PBAE-based nanoparticles carrying 4-1BBL and IL-12 DNA, particularly in combination with anti-PD-1 checkpoint blockade therapy, on tumor growth and animal survival is demonstrated. The mechanism of action of this technology also is explored using in vitro and in vivo assays to quantify the effects of tAPC reprogramming on the local and systemic immune system. Finally, it is shown that these results can be replicated in a second tumor, the MC38 colorectal carcinoma model, supporting the potential clinical utility of the technology.

### 2.4 Results

### 2.4.1 Selection of PBAE Transfection Agent for B16-F10 Cells In Vitro and In Vivo

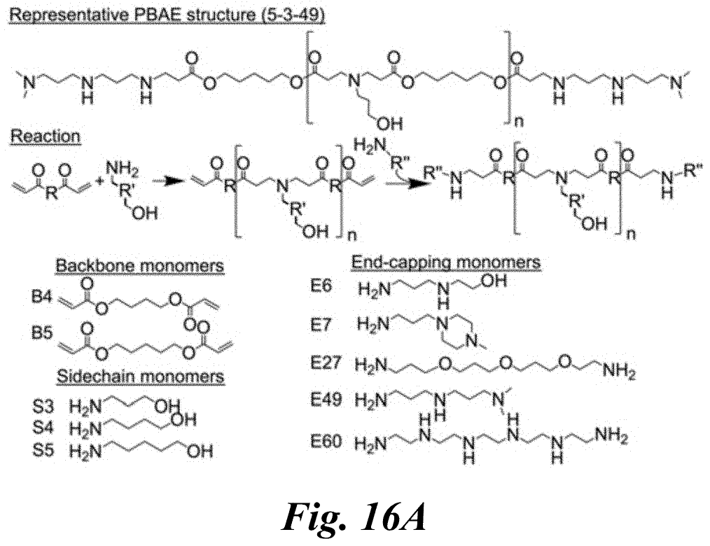

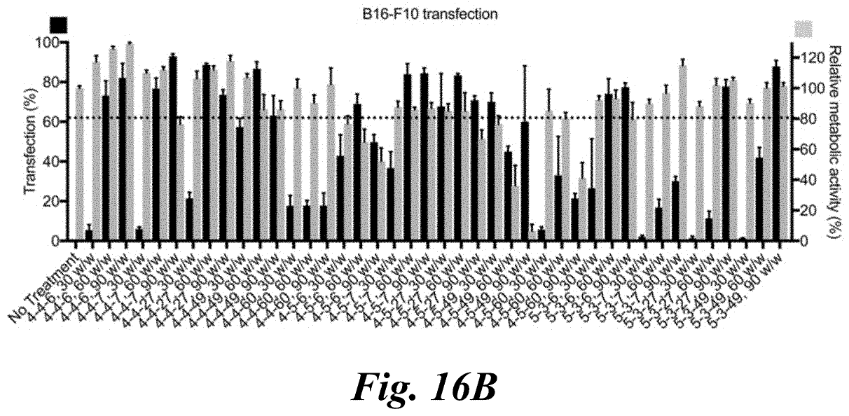

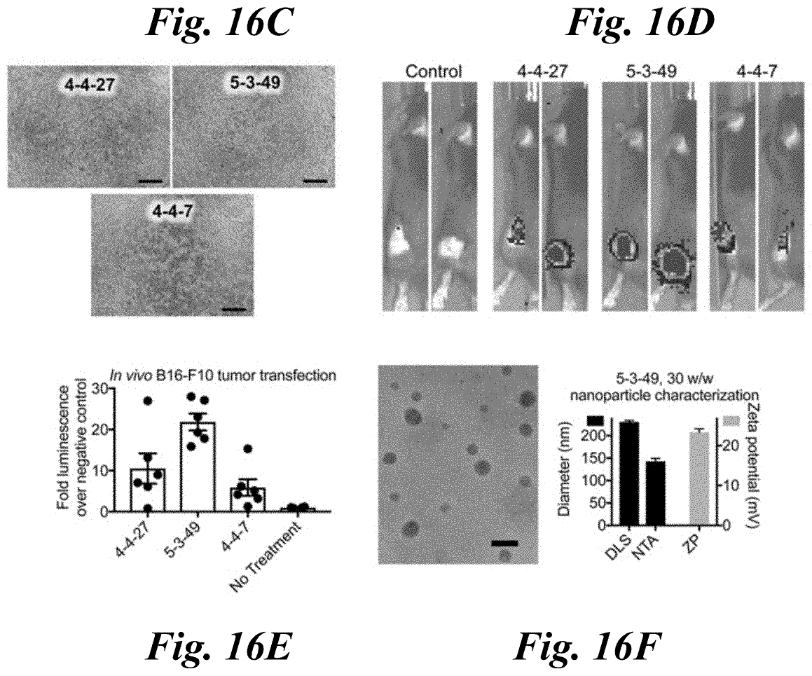

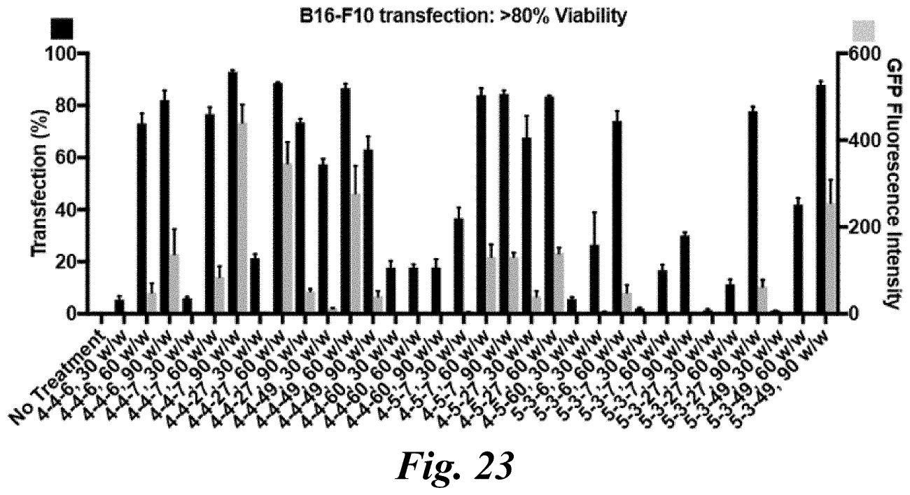

An array of PBAEs were synthesized based on structures previously shown to be safe and effective at transfecting various types of cancer cells with specificity over healthy cells, Tzeng et al., 2013; Guerrero-Cazares et al., 2014; Mangraviti et al., 2015. (FIG. 16A). B16-F10 cells were transfected in vitro with green fluorescent protein (GFP) DNA as a reporter gene to assess gene transfer efficacy. Among the nanoparticle formulations with low (<20%) toxicity, the top three polymers, named 4-4-7, 4-4-27, and 5-3-49, transfected 93.0±0.6%, 88.6±0.4%, and 88±2% of cells, respectively (FIG. 16B and FIG. 16C), with geometric mean fluorescence intensities of 440±40-fold, 350±50-fold, and 260±50-fold above the untreated control, respectively (FIG. 23). The top three PBAEs were then used to form nanoparticles with firefly luciferase (fLuc) DNA for in vivo transfection of subcutaneous (s.c.) B16-F10 tumors. After intra-tumoral (i.t.) injection of 5 μg of fLuc DNA in PBAE nanoparticles, the In Vivo Imaging System (IVIS) was used to measure gene expression in the tumor (FIG. 16D and FIG. 16E). PBAEs 4-4-7, 4-4-27, and 5-3-49 led to 11±9-, 22±5-, and 6±5-fold higher luminescence signal than control animals, and PBAE 5-3-49 was selected as the lead in vivo transfection agent for all further in vitro or in vivo studies on delivery of functional genes. Transmission electron microscopy (TEM) imaging showed that nanoparticles are approximately 50 to 100 nm when dry, and nanoparticle tracking analysis (NTA) and dynamic light scattering (DLS) measured a number-average hydrodynamic diameter of 143±6 nm and an intensity-weighted Z-average hydrodynamic diameter of 231±3 nm, respectively (FIG. 16F). As expected, due to a large ratio of cationic polymer to anionic DNA, nanoparticles were measured by electrophoretic motility to have a positive zeta potential of approximately +23.3±0.9 mV.

### 2.4.2 In Vitro Expression of 4-1BBL and IL-12 by Transfected B16-F10 Cells

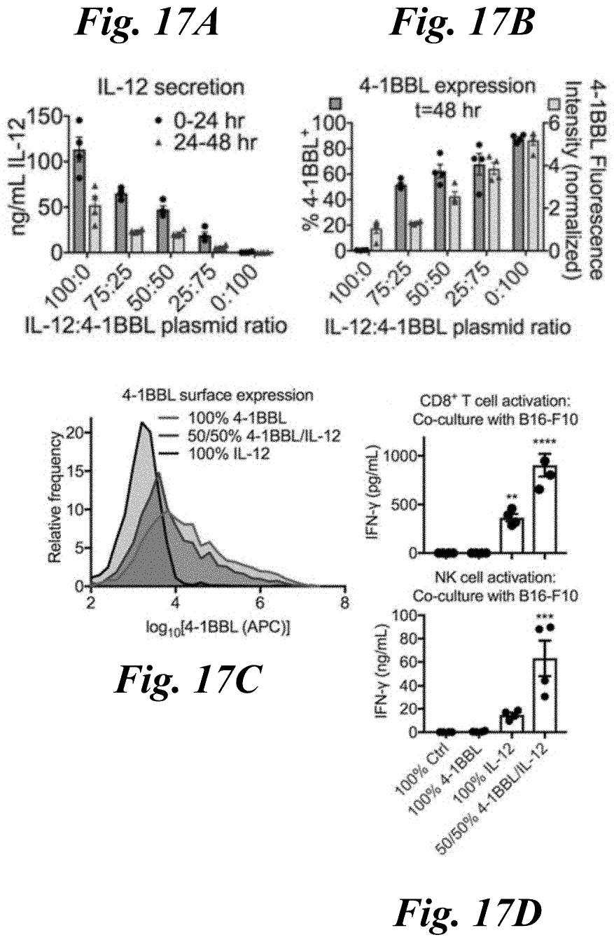

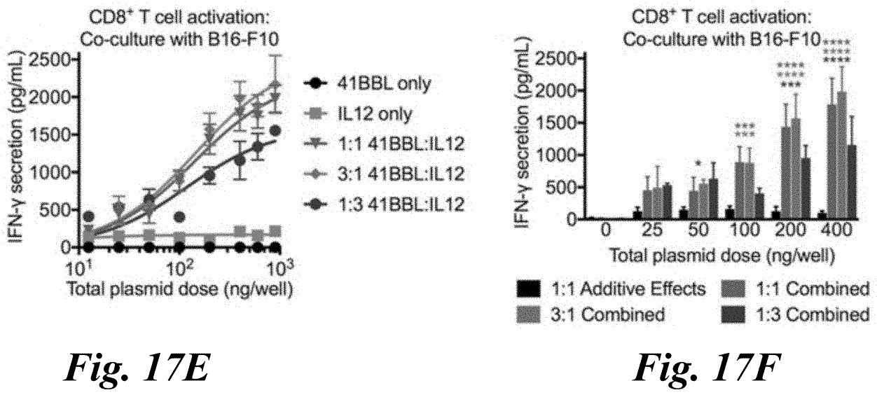

Using PBAE 5-3-49, B16-F10 cells were transfected in vitro with both 4-1BBL and IL-12, evaluating various ratios of the two plasmids. The supernatant was collected after 24 h and 48 h and measured by conformational enzyme-linked immunosorbent assay (ELISA) for IL-12 expression (FIG. 17A). IL-12 was detected by ELISA at both time points, with the most cytokine released within the first 24 h of transfection and approximately 50% less being released over the following 24 h. As expected, the amount of secreted IL-12 was tunable based on the 4-1BBL:IL-12 plasmid ratio, but, at all ratios tested, high IL-12 levels were detectable by ELISA. Expression of surface-bound 4-1BBL was measured by flow cytometry and was detected on transfected B16-F10 cells (FIG. 17B and FIG. 17C), with an increasing proportion of cells transfected (%4-1BBL+) and increasing amount of protein produced (normalized fluorescence intensity) as the ratio of 4-1BBL:IL-12 increased. Even at a 50:50 mass ratio of the two plasmids, 62±10% of cells were 4-1BBL+. In this condition, a total of 69±10 ng/mL IL-12 also was secreted over 48 h, corresponding to 69±10 ng from a starting number of 105 cells, an amount that surpasses concentrations shown to induce Th1 differentiation or expansion of cytotoxic T cells. Diaz-Montero et al., 2008. These results indicate the feasibility of co-complexing and co-delivering both plasmids at once to convert B16-F10 cells into tAPCs.

2.4.3 In Vitro Activation of Cytotoxic Lymphocytes by B16-F10 tAPCs

As a demonstration of the feasibility of this strategy for immune stimulation, B16-F10 cells were transfected in vitro with 4-1BBL and/or IL-12 to reprogram them into tAPCs. The tAPCs were then cocultured with primary CD8+ T cells or NK cells isolated from the spleens of C57BL/6 mice. After 18 h, the concentration of secreted interferon (IFN)-γ in the culture medium was measured by ELISA as a surrogate for T or NK cell activation (FIG. 17D). B16-F10 cells transfected with a control plasmid (fLuc) or with 4-1BBL alone elicited nearly undetectable levels of IFN-γ expression. Transfection with IL-12 alone elicited significantly higher but still low amounts of IFN-γ secretion, compared with the control. Transfection with both 4-1BBL and IL-12 in combination, however, showed strong synergy between the two signals, resulting in significantly greater IFN-γ secretion than the additive effect of the two genes on their own. Similar patterns were seen with both CD8+ T cells and NK cells, suggesting that this combination of signals 2 and 3 is suitable for activation of both types of cytotoxic immune cells. To more quantitatively assess the synergy between signals 2 and 3, B16-F10 cells were transfected in vitro with a wide range of doses of 4-1BBL alone, IL-12 alone, or combinations of the 4-1BBL and IL-12 plasmids at 1:3, 1:1, and 3:1 plasmid mass ratios. After coculture with primary CD8+ T cells, it was found that signals 2 and 3 alone are unable to strongly stimulate IFN-γ secretion, even at the highest doses tested, while treatment with the plasmid combinations resulted in the expected dose-response of IFN-γ secretion (FIG. 17E). When the effects (IFN-γ secretion) of 4-1BBL transfection alone and IL-12 transfection alone were added together, it was found that the effect of transfection with a matched total plasmid dose of 4-1BBL and IL-12 in combination was statistically significantly higher starting at a dose of 50 ng of plasmid per well for some combinations, and all combinations were significantly higher than the added effects of transfection with the individual plasmids at higher doses (FIG. 17F).

### 2.4.4 In Situ Genetic Reprogramming of B16-F10 Tumors for Anticancer Immunotherapy

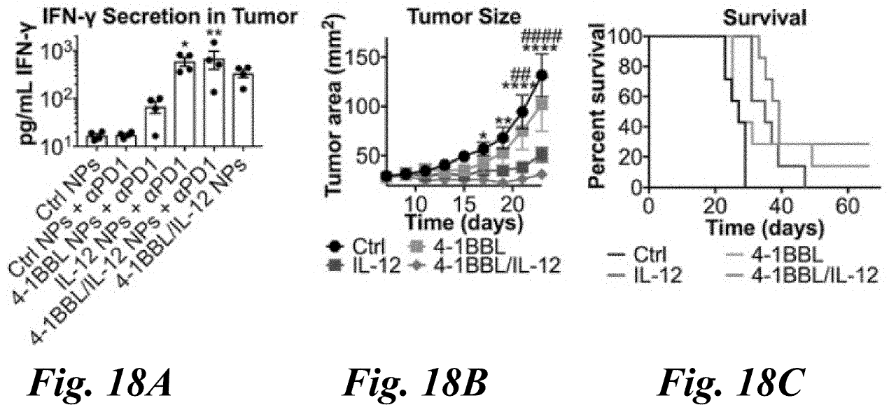

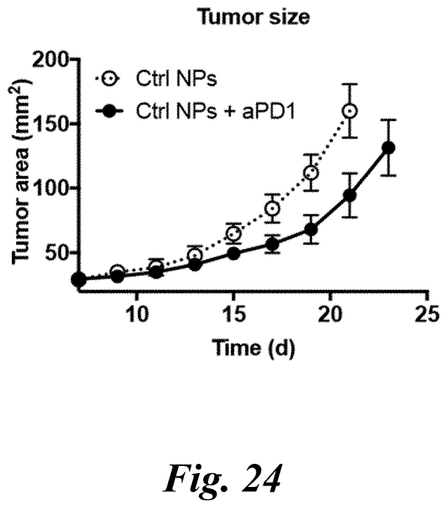

B16-F10 melanoma cells were inoculated s.c. in the flank of C57BL/6 mice. PBAE/DNA nanoparticles were injected i.t. on days 7, 9, and 11 after tumor inoculation, and anti-PD-1 monoclonal antibody was administered intraperitoneally (i.p.) on days 7 and 9. On day 14, 3 days after the final nanoparticle treatment, IFN-γ was measured in the tumor interstitial fluid (TIF) by ELISA in tumors treated with signal 2 and/or 3 nanoparticles (FIG. 18A). Results followed a similar trend to that seen in vitro, with IL-12 having a stronger effect than 4-1BBL alone and the combination of both signals 2 and 3 eliciting the greatest amount of IFN-γ secretion. Interestingly, although anti-PD-1 administration alone was not sufficient to elicit IFN-γ secretion, mice treated with both 4-1BBL/IL-12 nanoparticles and anti-PD-1 together had greater IFN-γ in the TIF than mice treated with nanoparticles alone. The tumors in mice treated with anti-PD-1 and control (fLuc) nanoparticles grew similarly (no statistically significant differences) to the tumors in mice treated with control nanoparticles alone (FIG. 24). Among mice treated with anti-PD-1 in the background, a gold-standard immunotherapy used in the clinic for advanced melanoma, tumor growth was significantly slower in mice treated with IL-12 nanoparticles than in controls, and this effect was even stronger in mice treated with 4-1BBL/IL-12 nanoparticles (P<0.0001 for both at t=23 d) (FIG. 18B). While 4-1BBL nanoparticles did not cause significantly slower tumor growth compared to the control over the time frame analyzed, this appears to be due to large variability in the response rate for this group. This is apparent from the survival curve (FIG. 18C), which shows that, while the tumor in most of the mice in the 4-1BBL nanoparticle group followed similar growth trends to those seen in the controls, a small number in this group survived substantially longer-one mouse cleared the flank tumor entirely, and no disease was apparent 66 d after the initial tumor implantation in this long-term survivor. By contrast, the IL-12 nanoparticle and 4-1BBL/IL-12 nanoparticle groups (median survival of 35 d and 39 d, respectively) survived significantly (P=0.0018) longer than the control group (median survival of 23 d), representing a 50 to 70% increase in median survival. A significant portion of the mice (28.6%) in the 4-1BBL/IL-12 nanoparticle group also cleared the tumor entirely and were disease-free at t=66 d.

### 2.4.5 Analysis of Local Immune Cells In Vivo Shows Cytotoxic Cellular Responses

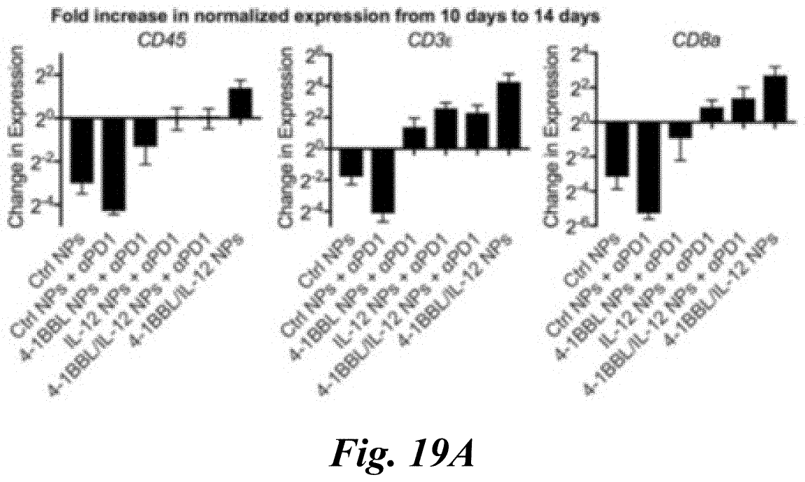

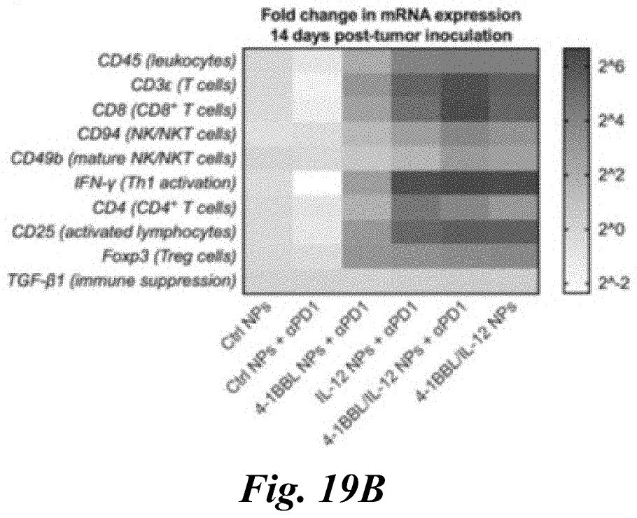

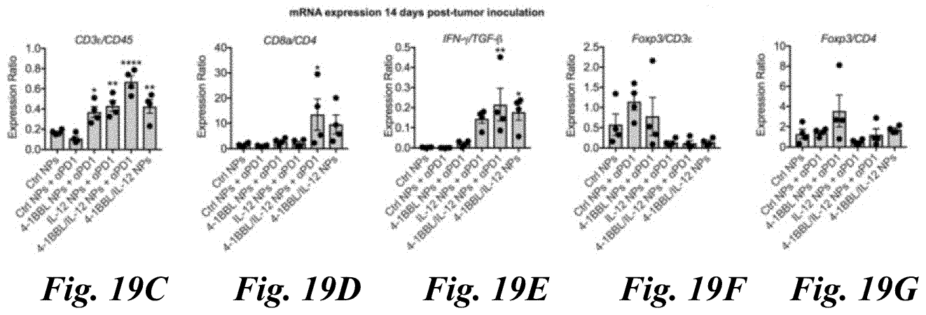

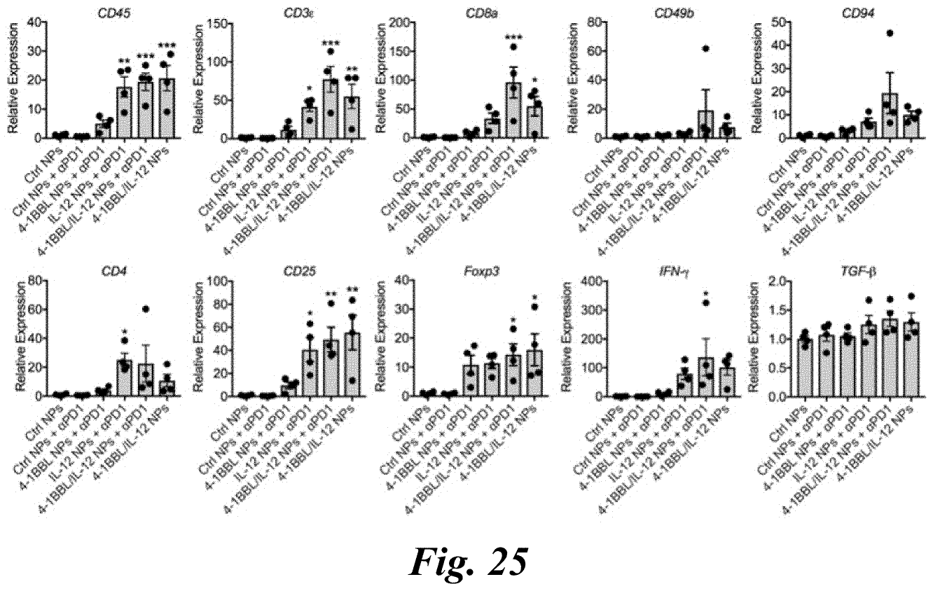

Tumors were excised for analysis by qPCR 10 and 14 days after tumor inoculation. Between those two time points, the relative expression of CD45, expressed by all leukocytes, and CD3c, expressed by all T cells, decreased in the groups treated with only control nanoparticles or control nanoparticles with anti-PD-1 checkpoint blockade therapy (FIG. 19A). By contrast, tumors treated with tAPC reprogramming nanoparticles encoding 4-1BBL and/or IL-12 had greater expression of CD45, CD3c, and CD8a at 14 d than at 10 d. This indicates that, in the control animals, the growth of malignant tumor cells outpaced the recruitment and expansion of immune cells; the opposite appears to be the case for animals treated with reprogramming nanoparticles, with IL-12 alone or in combination with 4-1BBL having the greatest effect. Other genes also were analyzed by qPCR after 14 d, and gene expression profiles were evaluated as an approximation of the types of immune cells present in the tumor (FIG. 19B). Tumor-infiltrating leukocytes (CD45), T cells (CD3ε)—particularly CD8+ T cells (CD8a)—and NK cells (CD94 and CD49b) were found to be elevated to the greatest extent in groups treated with IL-12 or 4-1BBL/IL-12 nanoparticles, either with or without additional anti-PD-1 treatment. The increase in IFN-γ expression in nanoparticle-treated groups followed the same trends seen via ELISA measurement of IFN-γ protein and is characteristic of a cytotoxic or Th1-biased immune response that could promote tumor control. This is in agreement with the increased expression of markers of cytotoxic lymphocytes. Interestingly, while the overall proportion of immune cells in all nanoparticle-treated tumors appears to be higher, the ratio of CD3ε to CD45 expression was highest in tumors administered 4-1BBL/IL-12 nanoparticles along with anti-PD-1, suggesting that the proportion of T cells among the infiltrating leukocytes was highest in that group (FIG. 19C). The ratio of CD8a to CD4 expression in this group also was significantly higher, again suggesting an immune response more biased toward a Th1 or antitumor cytotoxic phenotype (FIG. 19D), a result corroborated by the ratio of IFN-γ to TGF-β1 expression (FIG. 19E). Finally, while the CD3ε expression was elevated in 4-1BBL nanoparticle-treated groups compared to controls, measurements suggest that a greater proportion of T cells in some animals of this group have a regulatory phenotype (T regulatory cells [Tregs]), as indicated by Foxp3 expression (FIG. 19F and FIG. 19G). The ability of 4-1BB/4-1BBL signaling to induce either stimulatory or regulatory T cells under different conditions, Bartkowiak and Curran, 2015, may explain the large variability in results seen in mice administered nanoparticles encoding 4-1BBL alone, as some tumors in this group showed slowed growth or were eliminated entirely, while other tumors seemed to grow even more quickly than control tumors. By contrast, all of the groups treated with IL-12 or 4-1BBL/IL-12 nanoparticles showed decreased Foxp3 expression when normalized to CD3ε expression, demonstrating the importance of codelivery of a Th1-biased cytokine. Gee et al., 2009. Other details of qPCR analysis on lymphocyte-related markers are provided in FIG. 25.

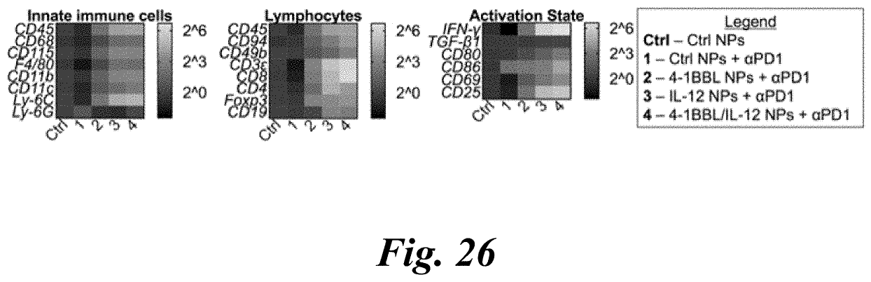

Additional qPCR analysis also was carried out to investigate the effects of this therapy on components of the innate immune system and markers of the cells' activation state (FIG. 26). Some monocyte, dendritic cell (DC), macrophage, and neutrophil markers are elevated in the 4-1BBL-only group, but less so than many of the lymphocyte markers tested. The general trend seen for lymphocytes also was observable for many of the innate immune cell and general infiltrating leukocyte markers, with slight up-regulation in tumors treated with 4-1BBL, greater up-regulation in tumors treated with IL-12, and the greatest effect in tumors treated with both. The difference among groups, however, was most striking for lymphocyte markers, particularly CD3ε (T cells) and CD8a (CD8+ T cells) and IFN-γ as an activation marker. The data further support the proposed mechanism of action as it highlights that a major effect of the treatment is on the recruitment, activation, and/or expansion of cytotoxic T lymphocytes as the technology was designed to target.

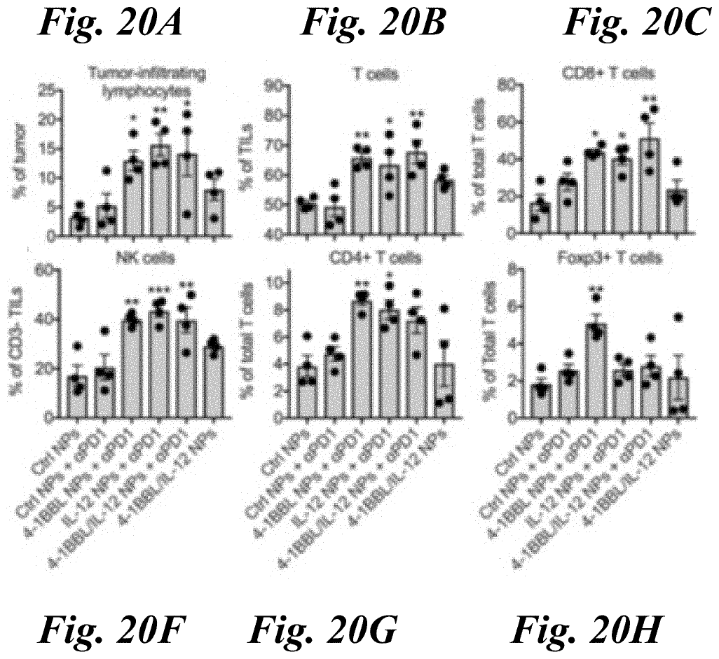

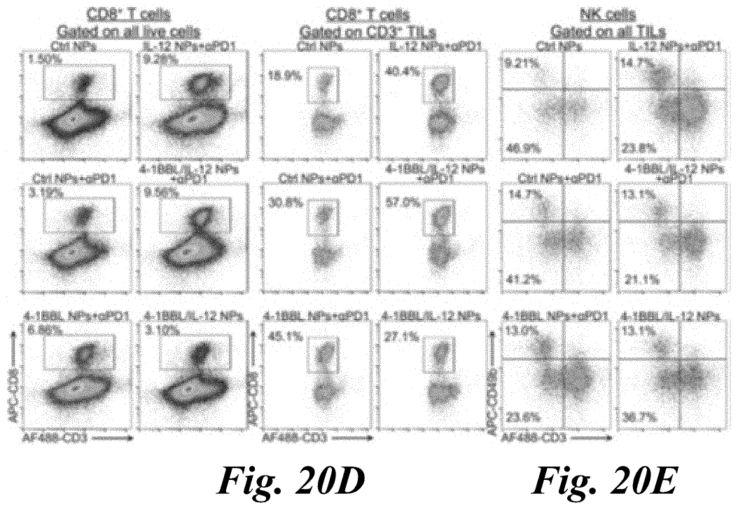

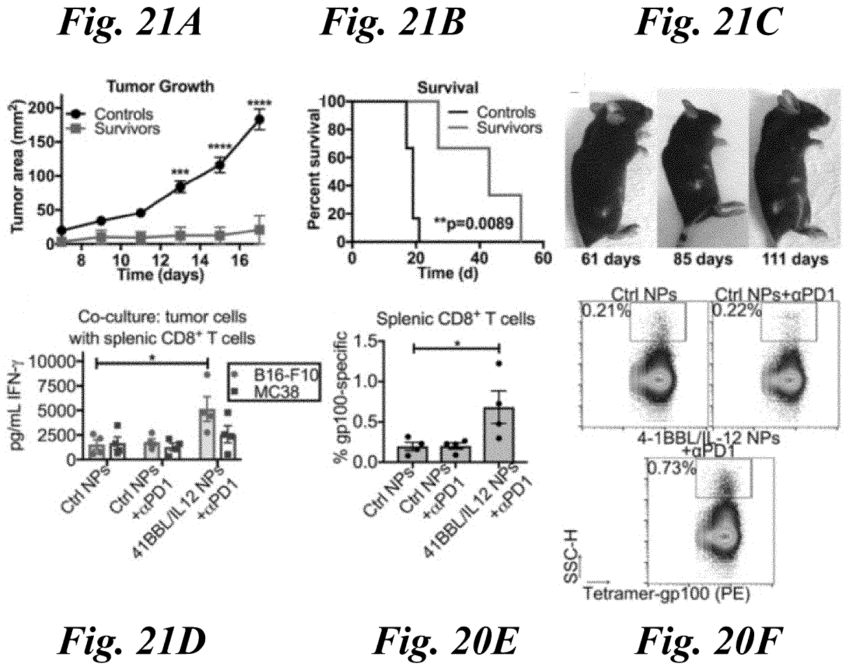

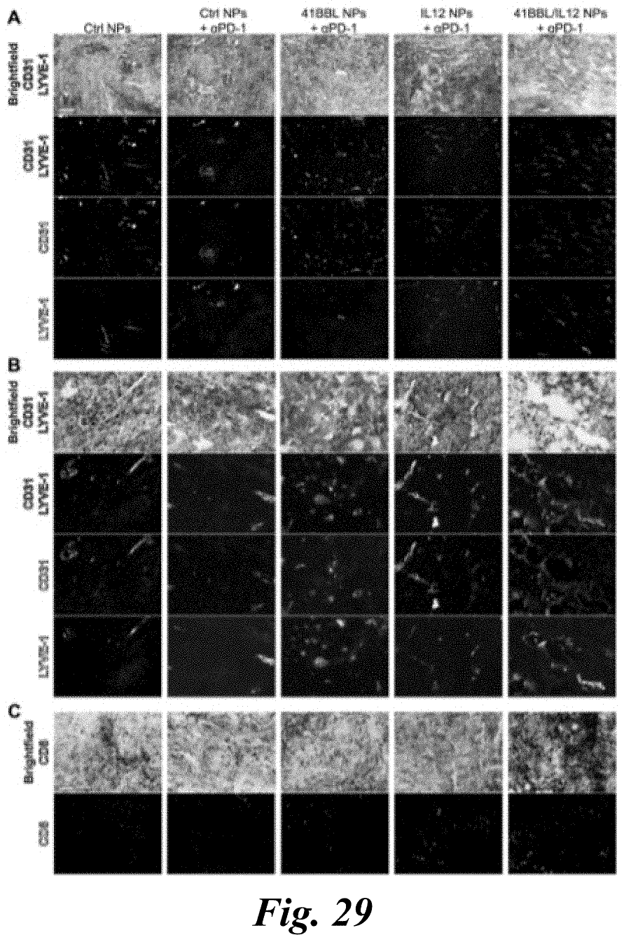

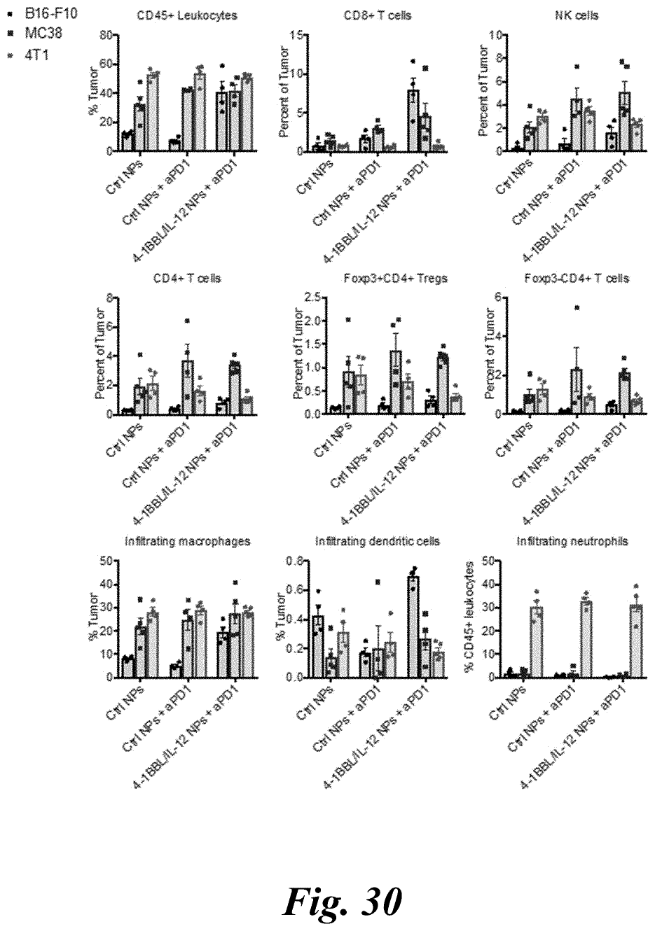

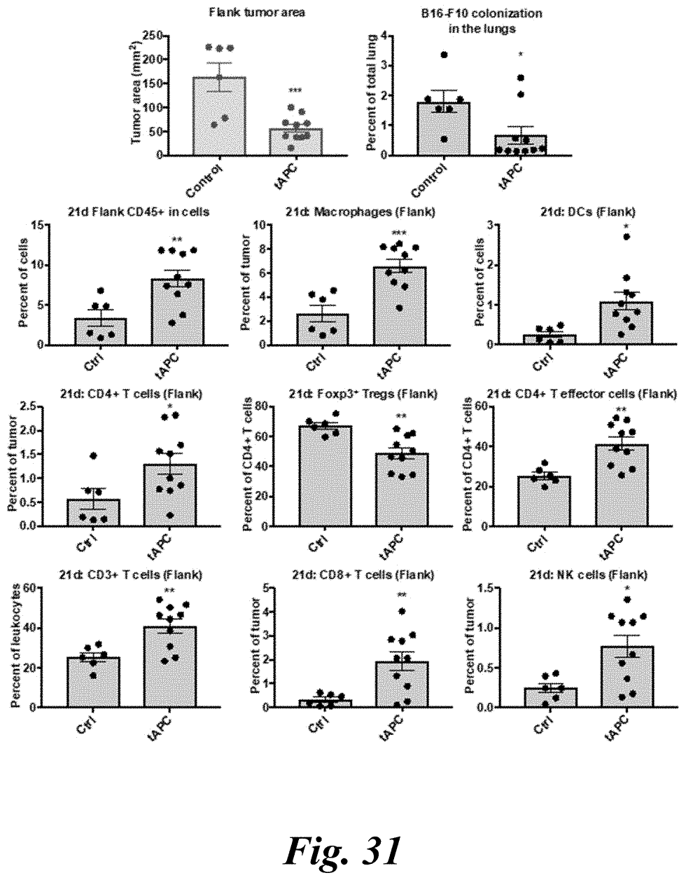

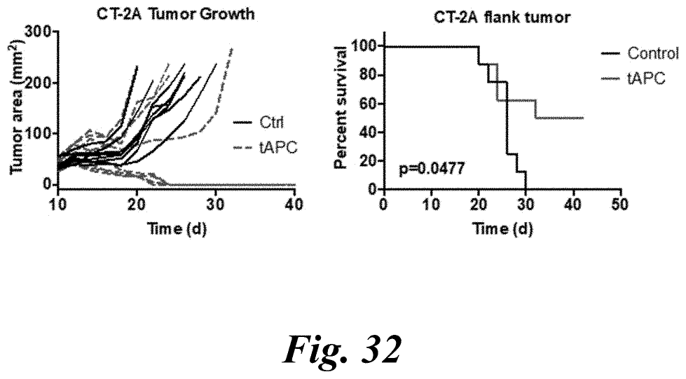

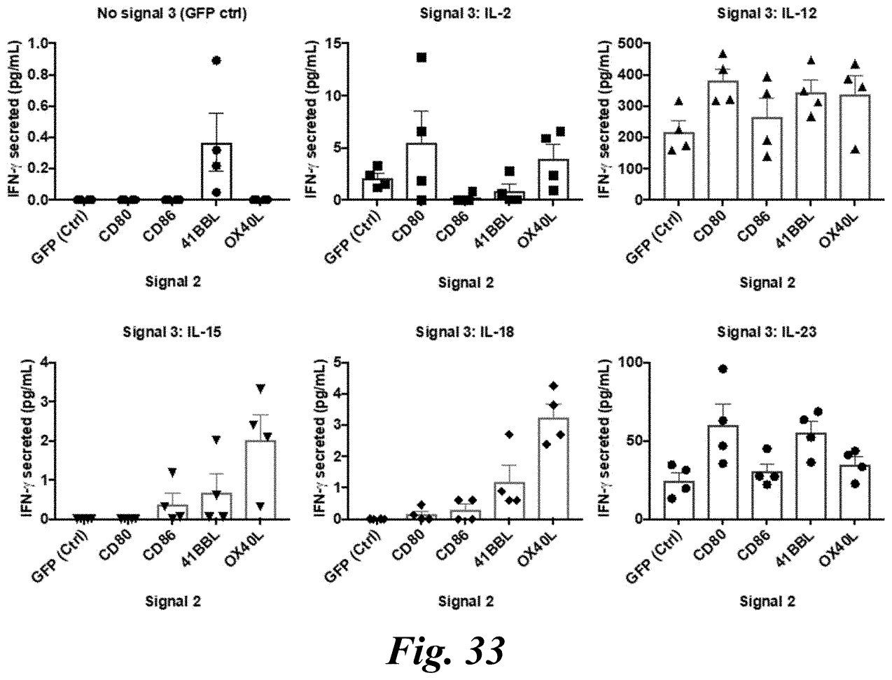

The messenger RNA (mRNA) expression results also were verified by flow cytometry, which yielded similar trends. Tumors were excised 14 d after inoculation (3 d after the final treatment), and groups treated with 4-1BBL, IL-12, or 4-1BBL/IL-12 nanoparticles along with anti-PD-1 showed a higher proportion of tumor-infiltrating lymphocytes (TILs) in the tumor, a greater proportion of which were T cells (FIG. 20A and FIG. 20B). Those groups, especially the 4-1BBL/IL-12 nanoparticle group, had the highest proportion of CD8+ cells among the CD3+ T cells, as well as the highest proportion of NK cells among CD3− TILs (FIG. 20C-FIG. 20F). While the CD4+ T cell population was elevated in some of the nanoparticle-treated groups, the proportion of Foxp3+ cells (Tregs) was highest in the 4-1BBL only nanoparticle group, concurring with qPCR results (FIG. 20G and FIG. 20H). Interestingly, the effect of checkpoint blockade therapy was clearer in the flow cytometry results where mice treated with only 4-1BBL/IL-12 nanoparticles but no anti-PD-1 showed fewer TILs, T cells, and cytotoxic cells than mice treated with both nanoparticles and anti-PD-1. Immunohistochemistry on the 14-d tumors qualitatively supports the flow cytometry and qPCR results, with CD8 expression showing the presence of cytotoxic T cells throughout the tumor and CD31 expression and LYVE-1 expression showing the presence of blood vessels and lymphatic vessels throughout the tumor (FIG. 29).

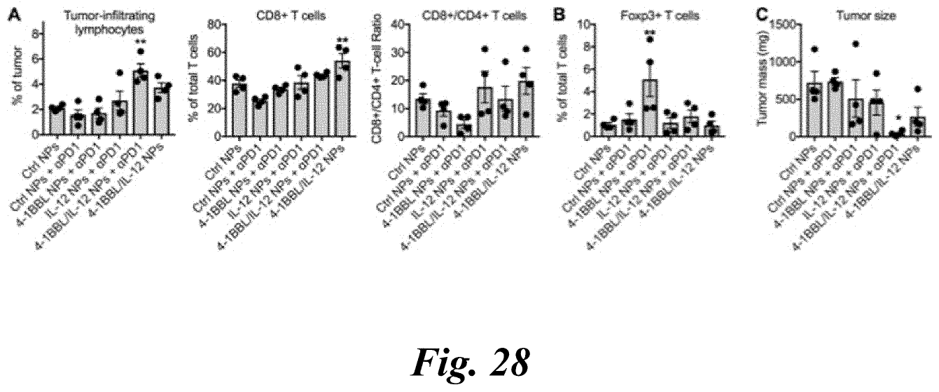

After 18 d, significantly more TILs were still present in the group treated with 4-1BBL/IL-12 nanoparticles and anti-PD-1. The proportion of CD8+ cells among total T cells and the ratio of CD8+/CD4+ T cells were still higher in groups treated with signal 3 or signal 2/3 nanoparticles although fewer of these differences were statistically significant than at 14 d (FIG. 21A). The prevalence of Tregs in the 4-1BBL-only nanoparticle-treated group remained high at 18 d, with the spread in the data showing variability of response within the group (FIG. 21B). Some of the greater variation at this time point may be due to the large differences in the size of the tumors. Tumors in the group treated with control nanoparticles, control nanoparticles and anti-PD-1, and 4-1BBL nanoparticles and anti-PD-1 had average masses of 700±200 mg, 730±50 mg, and 500±200 mg, respectively (mean±SE), and some of these tumors had grown large enough to require euthanasia of the mouse (FIG. 21C). By contrast, mice treated with 4-1BBL/IL-12 nanoparticles and anti-PD-1 all had significantly smaller tumors (40±20 mg), with relatively few cells that could be extracted for analysis. Other details of flow cytometry analysis are provided in FIG. 28.

2.4.6. Long-Term and Systemic Immunity Conferred by tAPC Nanoparticle Treatment

Some of the mice treated with either 4-1BBL or 4-1BBL/IL-12 nanoparticles and anti-PD-1 fully cleared their tumors and were considered long-term survivors when no disease was detectable after 50 d (two-fold longer survival than the longest surviving mouse in the control group) (FIG. 18C). At t=66 d post-tumor implantation, the long-term survivors were rechallenged with s.c. B16-F10 melanoma tumors on the opposite flank, along with age-matched, previously untreated controls. The tumor growth rate was significantly slower (P<0.0001 after t=15 d post-challenge) in the survivors than in controls (FIG. 21A). Although all mice did eventually grow tumors in the absence of any further treatment, the long-term survivors lived significantly longer (P=0.0089) than the age-matched controls (FIG. 21B), with >100% improvement in median survival time. The ability of these pretreated mice to resist the formation of a new tumor months later, particularly a tumor at a different location, is indicative of a long-lasting and systemic antitumor immune response. Of the long-term survivors, some developed a depigmented patch of fur on the flank where the original tumor had been implanted and then treated by local nanoparticle injection. This vitiligo-like pattern not only lasted throughout the course of the study but also began to spread to other patches of fur (FIG. 21C). Although this was an unintended side effect, the development of vitiligo has been found to correlate with positive responses to immunotherapy in human melanoma patients, Nakamura et al., 2017, and is another indication of a cytotoxic immune response specific to melanocytes, the cell type from which the cancer is derived. The later spread of depigmented patches on the mouse also is another indication that the cellular immune response to tAPC reprogramming treatment was not confined to the location where the nanoparticles were locally injected but rather had a systemic component, and the destruction of melanocytes further supports the role of cytotoxic T cells in the antimelanoma response. This finding was further evaluated by dissociating the spleens of tumor-bearing mice treated with either control nanoparticles with or without anti-PD-1 or reprogramming nanoparticles with anti-PD-1.

Splenic CD8+ T cells were isolated and cocultured with 4-1BBL/IL-12-transfected tumor cells in vitro to test for stimulation. It was found that CD8+ T cells from spleens of tAPC-treated mice were more activated by transfected B16-F10 cells in vitro, as measured by IFN-γ secretion (FIG. 21D). This indicates that splenic CD8+ T cells from tAPC-treated mice were either 1) generally more activated than CD8+ T cells from control mice or 2) enriched for tumor-specific T cells. Interestingly, although these T cells were slightly more activated by transfected MC38 cells compared to controls, this difference was not statistically significant. This suggests that the T cell response to B16-F10 cells is tumor-specific although there may be some shared epitopes with other tumor cell lines that cause a slight enhancement to the immune response to the other tumor cell line. The same CD8+ T cells were stained with a tetramer loaded with gp100, a common B16-F10 tumor antigen. Once again, cells isolated from animals treated with tAPC-reprogramming NPs had a higher proportion of gp100-specific T cells (FIG. 21E and FIG. 21F). It should be noted that the tAPC reprogramming strategy is not antigen-specific and is expected to generate an immune response directed against various different B16-F10 antigens so the data on only one particular antigen, gp100, while already encouraging, is likely to be an underestimation of the full effects of the treatment. Moreover, although the nanoparticle treatment is administered intratumorally, the effects on CD8+ T cells were measured in the spleen, showing that the antitumor cellular immune response is likely to be widespread in the body and not confined to the initial tumor and nanoparticle injection site.

2.4.7 Applicability of the tAPC Strategy to Other Tumor Types

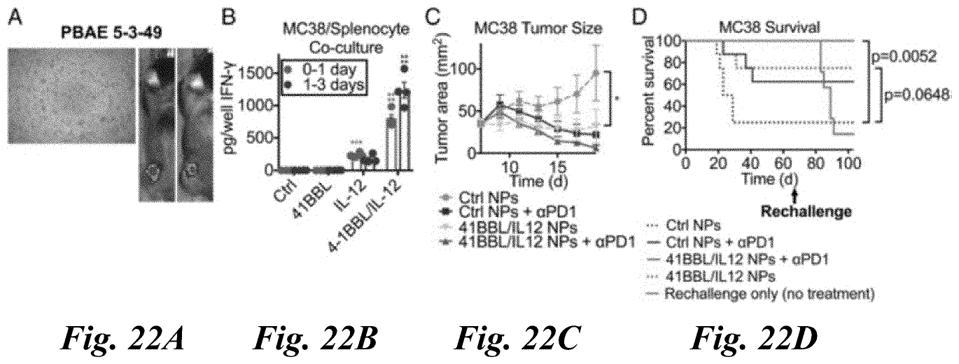

To support the hypothesis that this tumor reprogramming immunotherapy can have broad clinical applicability, this treatment was evaluated on the MC38 colorectal carcinoma tumor model. The same screening methods were used to identify leading PBAEs for MC38 transfection in vitro and in vivo after i.t. injection into an s.c. tumor (FIG. 22A). MC38 cells were transfected with 4-1BBL, IL-12, or both and cocultured with primary splenocytes, and the same trends in IFN-γ secretion were seen with MC38 as with B16-F10 after 1 to 3 d of coculture (FIG. 22B). An antitumor efficacy study was then carried out in MC38-bearing mice, and treatment with the nanoparticles was shown to be effective in slowing or preventing tumor growth (FIG. 22C). Excitingly, in this model, all mice in the group treated with 4-1BBL/IL-12 nanoparticles i.t. and anti-PD-1 antibody i.p. survived and fully cleared the initial tumor. Mice treated with 4-1BBL/IL-12 nanoparticles without anti-PD-1 also survived statistically significantly more than mice treated with control nanoparticles without anti-PD-1 (FIG. 22D). After 66 d, surviving mice were re-challenged with a second MC38 tumor injected s.c. on the opposite flank, along with naive, age-matched control mice that had not received a first tumor or any treatment. While almost 90% of control mice died due to tumor growth, none of the mice that had survived the first tumor developed the second tumor, indicating a very strong, long-lasting antitumor response at a site distant from the original treatment site.

### 2.5 Discussion

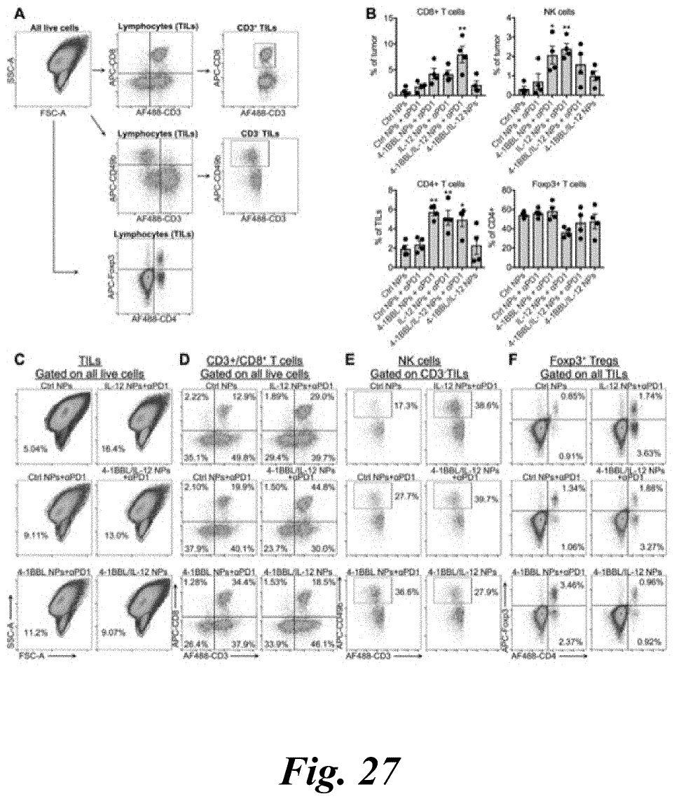

Despite promising preclinical and clinical results of cancer immunotherapy, further research and development are still needed to improve the efficacy and safety of such treatments and to decrease their cost and regulatory burden. Here, the challenging B16-F10 mouse melanoma model was used to demonstrate the therapeutic potential of a non-viral nanomedicine that can deliver immunostimulatory genes to a tumor and similarly strong results in a second murine model were achieved using MC38 colorectal carcinoma. Given the route of nanoparticle administration (i.t.), the focus of this technology is on solid tumors. In particular, this type of strategy could find clinical use in patients with solid tumors who have lesions that are accessible by needle or catheter but not easily operable. Additionally, it was shown that local nanoparticle delivery leads to a systemic and durable response, which may provide a method of harnessing the immune system to target metastases or invading malignant cells. In this approach, an endogenous cellular response is engendered without requiring ex vivo cellular manipulation. In contrast to other work describing the delivery of immunostimulatory agents to elicit an antitumor response, Emtage et al., 1999; Narvaiza et al., 2000; Nomura et al., 2001; Fan et al., 2017; Hanson et al., 2015; Cheng et al., 2018; and Zhu et al., 2017, it has been shown herein that co-expression of both signals 2 and 3 by signal 1-expressing tumor cells could cause activation of T cells in the direct context of tumor antigens. Thus, while this technology itself is independent of prior knowledge of tumor antigen expression profiles or MHC haplotype, it could activate an immune response specific to the patient and the tumor. Through antitumor efficacy studies, as well as analysis of immune cells in the tumor microenvironment, it has been shown that tAPC genetic reprogramming induces a cytotoxic, cell-mediated anticancer response. Not only were some of the treated animals able to clear their tumors, but they also resisted formation of new tumors at a distant location. Other studies have shown that tumor cells transfected ex vivo to express signals 2 and/or 3 are rejected by immunocompetent mice and lead to protective immunity, Chen et al., 1992; Townsend and Allison, 1993; Baskar et al., 1993; and Hiroishi et al., 1999, but the need for ex vivo cellular manipulation is a practical hurdle to this type of vaccination that dramatically reduced its accessibility. In contrast, PBAE/DNA nanoparticles can be an off-the-shelf therapy, able to provide a personalized endogenous cellular therapy response via a simple injection. Using PBAEs as DNA-delivery agents, strong in situ transfection of tumor cells was achieved using variants of materials that are safe and specific for cancer cells over healthy tissue, Tzeng et al., 2013; Guerrero-Cázares et al., 2014, thus preventing off-target activation of the immune system against healthy cells. PBAE-based nanoparticles for 4-1BBL and IL-12 transfection, therefore, can provide a safe, noninvasive, and easily manufactured technology for generating a potent therapeutic effect against tumors. This polymeric DNA nanoparticle system brings with it several advantages. While virus-mediated gene delivery can be highly effective, Guenther et al., 2014, their clinical use is hampered by their intrinsic immunogenicity, which can attenuate their activity and also lead to adverse side effects. They also are difficult to manufacture at scale, and their cargo capacity is limited, which may preclude the codelivery of multiple genes within the same particle. Other types of delivery vehicles, such as lipid nanoparticles (LNPs), are often constrained by high toxicity, Xue et al., 2014, or their own intrinsic immunogenicity, even without nucleic acid cargo. Vangasseri et al., 2006. Aside from their ability to transfect cancer cells, Tzeng et al., 2013; Guerrero-Cazares et al., 2014; Mangraviti et al., 2015; Zamboni et al., 2017; and Kim et al., 2016, PBAE nanoparticles can deliver dozens of plasmids within the same particle. Bhise et al, 2013; Wilson et al., 2017. This makes them ideal for the tAPC genetic reprogramming strategy as it has been shown here that signals 2 and 3 act synergistically on immune stimulation (FIG. 17). In particular, FIG. 19 and FIG. 20 demonstrate the importance of including both signals 2 and 3 in the treatment strategy as the combination of both not only led to the greatest effect but also decreased the chance of inadvertently stimulating undesirable cell types like Tregs. The presentation of surface-bound 4-1BBL signal 2 on malignant cells, ideally co-expressed alongside signal 1 antigens, focuses the immune response at the tumor site while the secretion of the IL-12 signal 3 helps to ensure a Th1 bias. The importance of this co-expression is particularly apparent in the results pertaining to mice treated with 4-1BBL nanoparticles in the absence of DNA encoding IL-12. While treatment with 4-1BBL alone was effective in some animals, in other mice, the tumor followed the same growth pattern as controls (FIG. 18) and even seemed to contain a higher proportion of Foxp3+ Tregs among TILs (FIG. 20 and FIG. 27). Because 4-1BBL can signal to Tregs, as well as CD8+ T cells, it is possible that overexpression of this signal 2 alone can stimulate an unwanted tolerogenic response rather than the desired cytotoxic antitumor response in some of the animals, which can be avoided with the codelivery of a cytokine like IL-12 that biases the immune system toward a Th1 antitumor response. The ease of combinatorial design of these particles, demonstrated in FIG. 16 and FIG. 23, and multiple previous reports, Tzeng et al., 2013; Guerrero-Cázares et al., 2014; Mangraviti et al., 2015; and Bhise et al., 2013, also imparts flexibility in the chemical properties of the polymer, the genes encoded by the DNA, and the association of polymer and DNA. While the signal 2 costimulatory molecule 4-1BBL and signal 3 cytokine IL-12 were found to be effective in this study, the PBAE/DNA nanotechnology provides a platform in which different polymers, as well as other immunostimulatory genes, can be easily used in a modular manner. While other nucleic acid delivery technologies have recently been described for use as immunotherapies, Hewitt et al., 2019, the underlying premise of which is supported by the findings, the use of plasmid DNA here rather than mRNA allows greater cargo stability and a simpler and less expensive manufacturing process, increasing the translatability of this work. Critically, a patient-specific response is engendered without requiring a priori knowledge of a patient's target antigens or ex vivo manipulation of a patient's cells. Finally, the ability of PBAE/DNA nanoparticles to be assembled in a simple protocol, be easily scalable, Wilson et al., 2017, and to be stored in a stable form for at least 2 years, Guerrero-Cazares et al., 2014, further supports the translational potential of this biotechnology and its possible broad impact on cancer patients.

### 2.6 Materials and Methods

### 2.6.1 Poly(Beta-Amino Ester) Synthesis

PBAEs were synthesized according to the reaction scheme in FIG. 16A as previously described, Tzeng et al., 2013, with each polymer consisting of one diacrylate “backbone” monomer (B), one amino alcohol “sidechain” monomer (S), and one amine-terminated “end-cap” monomer (E). Final PBAEs are named by a series of three hyphenated numerals, corresponding to the backbone, sidechain, and end-cap used in their synthesis, e.g., B4-S4-E7 is designated 4-4-7, and the like.

2.6.2.1 In Vitro Transfection of Cells: Screening with Reporter Gene

B16-F10 murine melanoma cells and MC38 murine colorectal cancer cells were a kind gift from Jonathan P. Schneck, Johns Hopkins University, Baltimore, Md. Both cell lines were cultured in complete growth medium consisting of RPMI 1640 (Gibco) supplemented with 10% fetal bovine serum (FBS) and 1% penicillin/streptomycin and were maintained at <80% confluency. The day before the transfection, cells were seeded in flat-bottom 96-well plates at 5×10⁴ cells per well in 100 μL of complete growth medium. On the day of transfection, nanoparticles were formed by diluting both green fluorescent protein (GFP) plasmid DNA (pEGFP-N1, purchased from Clontech and amplified by Elim Biopharmaceuticals [Hayward, Calif.]) and an array of PBAE polymers in 25-mM sodium acetate buffer, pH 5 (NaAc) and then mixing the diluted DNA and PBAEs to allow self-assembly. After 10 min, nanoparticles were added to the cells in complete growth medium at a final DNA concentration of 5 μg/mL and final PBAE concentrations ranging from 150 to 450 μg/mL The cells were incubated with nanoparticles at 37° C. and 5% CO₂ for 2 h, and then the media were replaced with 100 μL of fresh complete growth medium per well.

To assess toxicity of the PBAE/DNA nanoparticles, an MTS assay was carried out 24 h after transfection (CellTiter 96 Aqueous One Solution Cell Proliferation Assay, Promega, Madison, Wis.) to measure the metabolic activity of B16-F10 or MC38 cells. Transfection efficacy was assessed by flow cytometry 48 h after transfection, using an Accuri C6 flow cytometer (BD Biosciences, San Jose, Calif.) with a Hypercyt high-throughput attachment (IntelliCyt, Albuquerque, N. Mex.) and 1×phosphate-buffered saline (PBS) with 2% FBS as buffer. Transfection was measured as the percentage of total cells per well that were GFP+ as well as by geometric mean GFP fluorescence intensity. For both toxicity and transfection assays, PBAE/DNA nanoparticle-treated cells were compared to untreated cells as a control.

### 2.6.2.2 In Vivo Transfection of Tumors: Selection of PBAE Formulation Using Reporter Gene

All animal work described here was carried out in accordance with the guidelines set by the Johns Hopkins Animal Care and Use Committee. To select the top PBAE formulation, subcutaneous (s.c.) tumors were transfected with firefly luciferase (fLuc; pcDNA3-fLuc plasmid amplified by Aldevron, Fargo, N. Dak.) via intra-tumoral (i.t.) nanoparticle injection. The flanks of female C57BL/6J mice (The Jackson Laboratory, Bar Harbor, Me.) were shaved. Under anesthesia by isoflurane inhalation, 3×10⁵ B16-F10 cells in basal RPMI medium (without serum or antibiotics) were injected s.c. into each flank in 100 μL volume. For studies in MC38-bearing mice, tumors were established by shaving the flanks of mice and injecting 5×10⁵ cells s.c. into each flask in 100 μL of basal RPMI medium. After 7 d, when tumors had become palpable, mice were again anesthetized under isoflurane. PBAE/DNA nanoparticles were formed as described above using sodium acetate buffer at pH 7 to prevent excessive acidification of the tissue environment and injected i.t. in 25 μL volume. Due to the increased concentration, nanoparticles were all tested at a 30:1 wt/wt ratio of polymer to DNA, with a final DNA dose of 5 μg per tumor. Each tumor was considered a separate replicate. After 24 h, mice were injected intraperitoneally (i.p.) with 150 mg/kg d-luciferin (potassium salt solution in 1×PBS; Cayman Chemical Company, Ann Arbor, Mich.). After 8 min, mice were imaged by the In Vivo Imaging System (IVIS Spectrum; PerkinElmer, Shelton, Conn.) to measure bioluminescence. All mice were euthanized before the combined tumor area of both tumors exceeded 200 mm² measured by calipers. The nanoparticle formulation leading to the highest fLuc bioluminescence signal in both tumor models, PBAE 5-3-49 at 30 wt/wt mass ratio to DNA, was used for all future in vivo studies.

### 2.6.3 In Vitro T and NK Cell Stimulation by Transfected B16-F10 Cells

B16-F10 cells were seeded in 96-well plates and transfected as described above with PBAE/DNA nanoparticles encoding fLuc (control), 4-1BBL, IL-12, or a mixture of 4-1BBL and IL-12 at a 1:1 plasmid mass ratio. The next day, 8- to 12-wk-old female C57BL/6J mice were euthanized by CO₂ asphyxiation. Their spleens were removed and dissociated by pressing through a 40-μm cell strainer and washing with excess cold 1×PBS. The cells were pelleted by centrifugation at 300 relative centrifugal force for 5 min at 4° C., and the supernatant was removed. Red blood cells were lysed by resuspending the pellet in 1 mL of ACK lysing buffer (Quality Biological, Gaithersburg, Md.) for 1 min at room temperature, then diluting in 10 mL of cold 1×PBS. The cell suspension was centrifuged again at 300 rcf for 5 min at 4° C., the supernatant was removed, and the pellet was resuspended in MACS running buffer (1×PBS with 0.5% bovine serum albumin [BSA] and 2 mM EDTA). Cells were labeled with microbeads for magnetic negative isolation of CD8+ T cells or NK cells according to the manufacturer's instructions (Miltenyi Biotec, Auburn, Calif.) using MACS separation columns. The isolated CD8+ T cells or NK cells were then resuspended at 2×10⁶ cells per milliliter in complete RPMI growth medium and added directly to the plate of transfected B16-F10 cells (10⁵ lymphocytes in 50 μL added per well). After 18 h of incubation at 37° C. and 5% CO₂, the media in the wells were collected and measured by IFN-gamma (IFN-γ) ELISA (mouse IFN gamma uncoated ELISA; Invitrogen/Thermo Fisher Scientific, Carlsbad, Calif.). Differences in IFN-γ secretion among groups were detected by one-way ANOVA with Dunnett post-tests against the control (tumor cells transfected with fLuc control plasmid and cocultured with T or NK cells). Differences were considered statistically significant for P<0.05.

2.6.4 In Vivo Antitumor Efficacy of tAPC Reprogramming Nanoparticles in B16-F10 Model

Female 9-wk-old C57BL/6J mice were inoculated s.c. with 3×10⁵ B16-F10 cells on the right flank following the procedures described above. After 7 d, the area (width×length) of each tumor was measured by caliper, and the animals were ranked according to tumor size. Mice were assigned to each group, ensuring that all groups started with statistically equivalent mean tumor sizes. The experimenter administering treatments and measuring tumor size over time was blinded to group assignments. On days 7, 9, and 11 after tumor inoculation, PBAE nanoparticles with DNA encoding fLuc (control), 4-1BBL, IL-12, or a 1:1 mixture of 4-1BBL and IL-12 were injected i.t., with a final DNA dose of 5 μg in 25 μL per injection, as described above. On days 7 and 9, mice also were injected i.p. with 200 μg and 100 μg of monoclonal antibody against mouse PD-1, respectively (clone RMP1-14; BioXCell, West Lebanon, N.H.) or 1×PBS alone as a control. Tumor area was measured every 2 d after the start of treatment, and mice were euthanized when tumor area reached or exceeded 200 mm². Each group included n=7 mice. Differences in tumor size over time among mice treated with nanoparticles and anti-PD-1 were detected by two-way repeated-measures ANOVA with Dunnett post-tests against the control group (control fLuc nanoparticles administered i.t. and anti-PD-1 administered i.p.). Differences were considered statistically significant for P<0.05. Mice that cleared their tumor and survived with no apparent disease to t=50 d (two-old greater survival time than the last surviving mouse in the control group) were considered long-term survivors. Long-term survivors were rechallenged on the opposite (left) flank with an injection of 3×10⁵ B16-F10 cells at t=66 d following the initial tumor inoculation following the procedures described above. Naive, untreated, age-matched (18-wk-old) female C57BL/6J mice were inoculated with the same number of B16-F10 cells at the same time as controls. No further treatment was administered to any of the mice. Tumor size was measured over time by caliper, and survival was recorded. Differences in tumor size over time between the two groups were detected by t-tests with Holm-Sidak corrections for multiple comparisons. Differences were considered statistically significant for P<0.05. Differences in survival curves were detected by Mantel-Cox log-rank tests, with a Bonferroni correction for multiple comparisons.

### 2.6.5 Statistics

Unless otherwise specified, differences between two groups were calculated using Student's t-tests, with Holm-Sidak corrections for multiple comparisons where necessary. Differences among multiple groups at a single time point were calculated using one-way ANOVAs with Dunnett post-tests against the control group specified in each section above. Differences among multiple groups across multiple time points were calculated using two-way repeated-measures ANOVAs with Dunnett post-tests against the control group. The normality of data distributions was verified by Shapiro-Wilk tests.

### 2.7 Supporting Materials and Methods

### 2.7.1 Detailed Poly(Beta-Amino Ester) (PBAE) Synthesis

Backbone B4 (1,4-butanediol diacrylate), sidechains S3 (3-amino-1-propanol) and S5 (5-amino-1-pentanol), and end-cap E7 [1-(3-aminopropyl-4-methylpiperazine)] were purchased from Alfa Aesar (Tewksbury, Mass.). B5 (1,5-pentanediol diacrylate) was purchased from Monomer-Polymer and Dajac Labs (Ambler, Pa.) and S4 (4-amino-1-butanol) from Fisher Scientific (Hampton, N.H.). E6 [2-(3-aminopropylamino)ethanol], E27 (4,7,10-trioxa-1,13-tridecanediamine), and E49 (N,N-dimethyldipropylenetriamine) were purchased from Sigma Aldrich (St. Louis, Mo.), and E60 (pentaethylenehexamine) was purchase from Santa Cruz Biotechnology (Dallas, Tex.). All other chemicals used were anhydrous and reagent-grade.

Briefly, one backbone (B) monomer was polymerized with one sidechain (S) monomer at a 1.1:1 molar ratio of acrylates to primary amines in a neat solution at 90° C. for 24 hr. The resulting diacrylate-terminated base polymer was then reacted with an excess of an end-cap (E) monomer in anhydrous tetrahydrofuran (THF) at room temperature for 1 hr. The end-capped polymer was isolated by precipitation into anhydrous diethyl ether and collected by centrifugation at 3200 rcf for 5 min at 4° C. The supernatant was decanted and the polymer washed twice with ether, using centrifugation after each wash to pellet the polymer. The resulting product was dried under vacuum for 48 hr at room temperature, then dissolved in anhydrous dimethyl sulfoxide (DMSO) and stored as a 100 mg/mL solution at −20° C. with desiccant until use.

2.7.2 In Vitro Transfection of B16-F10 and MC38 Cells: Screening with Reporter Gene

B16-F10 or MC38 cells were cultured in complete growth medium consisting of RPMI 1640 (Gibco) supplemented with 10% fetal bovine serum (FBS) and 1% penicillin/streptomycin and were maintained at <80% confluency. The day before the transfection, cells were seeded in flat-bottom 96-well plates at 5×10⁴ cells/well in 100 complete growth medium. On the day of transfection, nanoparticles were formed by diluting green fluorescent protein (GFP) plasmid DNA (pEGFP-N1, purchased from Clontech and amplified by Elim Biopharmaceuticals, Hayward, Calif.) and an array of PBAE polymers in 25 mM sodium acetate buffer, pH 5 (NaAc) and then mixing the diluted DNA and PBAEs to allow self-assembly. After 10 min, nanoparticles were added to the cells in complete growth medium at a final DNA concentration of 5 μg/mL and final PBAE concentrations ranging from 150-450 μg/mL. The cells were incubated with nanoparticles at 37° C. and 5% CO₂ for 2 hr, and then the media were replaced with 100 fresh complete growth medium per well. To assess toxicity of the PBAE/DNA nanoparticles, an MTS assay was carried out 24 hr after transfection (CellTiter 96 Aqueous One Solution Cell Proliferation Assay, Promega, Madison, Wis.) to measure the metabolic activity of B16-F10 or MC38 cells. Transfection efficacy was assessed by flow cytometry 48 hr after transfection, using an Accuri C6 flow cytometer (BD Biosciences, San Jose, Calif.) with a Hypercyt high-throughput attachment (IntelliCyt, Albuquerque, N. Mex.) and 1×PBS with 2% FBS as buffer. Transfection was measured as the percentage of total cells per well that were GFP+ as well as by geometric mean GFP fluorescence intensity. For both toxicity and transfection assays, PBAE/DNA nanoparticle-treated cells were compared to untreated cells as a control.

### 2.7.3 In Vitro Expression of 4-1BBL and IL-12 by B16-F10 Cells

To ensure that exogenous signals 2 and 3 could be expressed by B16-F10 cells after transfection, nanoparticles were formed as described above for in vitro transfection using PBAE 5-3-49, the lead polymer for in vivo transfections. Following the results of in vitro screenings, the polymer was combined with DNA, at a mass ratio of 90 w/w. Cells were seeded in 96-well plates as described above and transfected with nanoparticles carrying DNA encoding fLuc (control), 4-1BBL, IL-12, or a mixture of 4-1BBL and IL-12 at plasmid mass ratios of 1:3, 1:1, and 3:1. The total amount of DNA in each nanoparticle formulation was the same, and 600 ng DNA was added per well for transfection. To measure secreted IL-12 expression, the B16-F10 culture medium was collected after 24 hr and 48 hr and measured by mouse IL-12 ELISA (ELISA MAX Deluxe kit, BioLegend, San Diego, Calif.). To measure 4-1BBL surface expression, transfected cells were trypsinized and stained for mouse 4-1BBL [phycoerythrin (PE)-labeled antibody against mouse 4-1BBL, clone TKS-1, BioLegend; 1:80 dilution] or an isotype control (PE-labeled rat IgG2a,κ isotype control antibody, BioLegend; 1:80 dilution) in 1×PBS with 2% FBS. The stained cells were washed twice, then analyzed by flow cytometry (Accuri C6 with Hypercyt attachment).

### 2.7.4 In Vitro Immune Stimulation by Transfected MC38 Cells

For studies with colorectal carcinoma, MC38 cells were seeded in 96-well plates and transfected as described above with plasmids encoding fLuc (control), 4-1BBL, or IL-12 or a combination of the 4 1BBL and IL-12 plasmids. The next day, splenocytes were isolated from nine-week-old female C57BL/6J mice, red blood cells were lysed, and splenocytes were resuspended in complete RPMI growth medium as described. To each well of transfected MC38 cells, 10⁵ splenocytes in 50 μL medium were added and co-cultured for 18 hr or 3 days. The secreted IFN-γ was quantified in the supernatant by ELISA, as described for the B16-F10 model.

### 2.7.5 Immunohistochemistry on B16-F10 Tumors

B16-F10 tumors were established subcutaneously (s.c.) in the right flank of C57BL/6 mice as described in Methods. After 7, 9, and 11 days, mice were treated by intra-tumoral (i.t.) injection of nanoparticles as described, following the groups used for the survival study. On days 7 and 9, mice also were treated with intraperitoneal (i.p.) injection of anti-PD-1 antibody where specified, as described previously. On t=14 days, mice were euthanized by cervical dislocation, and their tumors were removed, flash-frozen in a bath of dry ice and isopropanol, and stored at −80° C. until use. Before cryosectioning, tumors were mounted in optimal cutting temperature (OCT) medium, and 20-μm sections were adhered to Superfrost Plus slides (Thermo Fisher). Sections were allowed to dry at room temperature (RT) for approximately 30 min, then stored at −80° C. until staining.

For immunohistochemistry (IHC), sections were fixed for 5 min in cold acetone and allowed to dry at RT. Sections were then rehydrated in 1×PBS at RT for 10 min and blocked for 1 hr at RT in 1×PBS with 3% normal goat serum (NGS), 1% bovine serum albumin (BSA), and 0.3% TritonX-100. Slides were then stained either for CD8 or for CD31 and LYVE-1 using the primary antibodies listed in Table 3. Antibodies were diluted in carrier solution (1×PBS with 3% NGS and 0.3% TritonX-100) and incubated with the slides for 2 hr at room temperature in a humidified box. The slides were then washed in 1×PBS four times for 5 min each, then incubated with the secondary antibodies described in Table 3. Slides with anti-CD8 were probed with AF405-labeled anti-rabbit secondary antibodies; slides with anti-CD31 and anti-LYVE-1 were probed with AF405-labeled anti-rat and AF488-labeled anti-rabbit antibodies, respectively. After 1 hr of incubation at room temperature in a humidified box, protected from light, the slides were washed with 1×PBS four times for 5 min each. Coverslips were mounted on the slides in a mixture of 90% glycerol and 10% 1×PBS. Slides were sealed with clear nail polish, stored at 4° C. until use, and then imaged by fluorescence microscopy (Axio Observer.Z1, Zeiss).

TABLE 3
Antibodies used for immunohistochemistry

| Protein | | Host | | | | Catalog |
|---|---|---|---|---|---|---|
| target | Conjugate | species | Clone | Dilution | Supplier | number |
| CD8a | None | Rabbit | Polyclonal | 1:100 | Thermo | PA5-79011 |
| LYVE-1 | None | Rabbit | Polyclonal | 1:200 | Thermo | PA5-19620 |
| CD31 | None | Rat | 390 | 1:50 | BioLegend | 102402 |
| Rabbit IgG | Alexa Fluor 488 (AF488) | Goat | Polyclonal | 1:500 | abcam | ab150077 |
| Rat IgG | Alexa Fluor 405 (AF405) | Goat | Polyclonal | 1:200 | abcam | ab175671 |
| Rabbit IgG | Alexa Fluor 405 (AF405) | Goat | Polyclonal | 1:200 | abcam | ab175652 |

2.7.6.1 Assessment of Local Immune Response: IFN-γ Secretion into Tumor Interstitial Fluid

Mice were inoculated s.c. with 3×10⁵ B16-F10 cells on the right flank. At t=7 days, mice were treated with nanoparticles and/or anti-PD-1 was described above. At t=14 days, 7 days after initiating treatment and 3 days after the final nanoparticle treatment, n=4 mice per group were euthanized. The tumors were excised and cut into pieces of 2 mm or smaller, weighed, and resuspended in ELISA diluent buffer from the mouse IFN gamma uncoated ELISA kit (Invitrogen/Thermo Fisher). The tissue was incubated at 37° C. for 1 hr, then centrifuged at 300 rcf for 5 min, and the supernatant was removed and measured by IFN-γ ELISA according to the manufacturer's instructions. Differences in IFN-γ secretion among groups were detected by one-way ANOVA with Dunnett post-tests against the control (i.t. control nanoparticle administration only). Differences were considered statistically significant for p<0.05.

2.7.6.2 Assessment of Local Immune Response: qPCR

Mice were inoculated with B16-F10 s.c. flank tumors and treated as described above. After 10 and 14 days, or 3 days after the start of treatment and 3 days after the final treatment, n=4 mice per group were euthanized. Their tumors were excised, flash frozen in liquid nitrogen, crushed with pestles, and dissolved in TRIzol reagent (Invitrogen/Thermo Fisher). RNA was isolated according to the manufacturer's protocol and converted to cDNA using the High-Capacity cDNA Reverse Transcription Kit (Applied Biosystems/Thermo Fisher, Foster City, Calif.). cDNA was amplified using Power SYBR Green PCR Master Mix (Applied Biosystems/Thermo Fisher) and a StepOnePlus Real-Time Polymerase Chain Reaction (RT-PCR) System (Applied Biosystems). Expression levels of the genes listed in Table 4 were calculated by the delta CT method using beta-actin (ACTB) as a reference gene. Differences in relative expression levels were detected by one-way ANOVA with Dunnett post-tests against the control (i.t. control nanoparticle administration only). Differences were considered statistically significant for p<0.05. Normality of the distributions was confirmed by Shapiro-Wilk tests.

TABLE 4
Primers used for qRT-PCR

| | Accession | | |
|---|---|---|---|
| Gene | Number | Forward Primer (5′-3′) | Reverse Primer (5′-3′) |
| β-actin | NM_007393 | GGCTGTATTCCCCTCCATCG | CCAGTTGGTAACAATGCCATGT |
| CD3ε | NM_007648 | ATCACTCTGGGCTTGCTGAT | TGGGCTCATAGTCTGGGTTG |
| CD4 | NM_013488 | AAGAGGAGGTGGAGTTGTGG | GTTTGCACTCTGTCAAGGGG |
| CD8a | NM_009857 | AGCCCACCTTCGTTGTCTAT | AGCCTTCGTTTTCCTTGCTG |
| CD25 | NM_008367 | ACACCACCGATTTCTGGCTA | AGTCTGTGGTGGTTATGGGG |
| Foxp3 | NM_001199347 | ACCTATGCTACCCTTATCCG | CCGAACATGCGAGTAAACCA |
| CD49b | NM_008396 | AGCCCTGTCAGACATCAACA | TGGGAGTACTTGGTGCGAAT |
| CD94 | NM_010654 | GAATGCTGTGTTTGCCTGGA | TCTGGATTGGGGCTGAAGAA |
| IFN-γ | NM_008337 | CATGGCCTGTTTCTGGCTGTT | TCCTTTTGCCAGTTCCTCCA |
| TGF-β | NM_011577 | ACGGAATACAGGGCTTTCGA | CCGGTTCATGTCATGGATGG |
| CD45 | NM_001111316 | CCCGGGATGAGACAGTTGAT | ATTCTGCGCACTTGTTCCTG |
| CD68 | NM_001291058 | CCACAGTTTCTCCCACCACA | GTGTAGTTCCCAAGAGCCCC |
| CD115 | NM_001037859 | GGTTGTAGAGCCGGGTGAAA | TCTTGTGGTCAGGGTGCTTC |
| F4/80 | NM_010130 | ACCTGTAAACGGAGGCTTCCTG | CTGAGTTAGGACCACAAGGTGAG |
| CD11b | NM_008401 | AGTGCTGGGAGACGTGAATG | GCACTGAGGCTGGCTATTGA |
| CD11c | NM_021334 | GCGTGGAGAACTTTGATGCTTT | TACTGCTGCTTGGTGTCTCTG |
| Ly-6C | NM_010741 | CTTCTTGTGGCCCTACTGTGT | TTGGCACTCCATAGCACTCG |
| Ly-6G | NM_001310438 | CCTGAGACTTCCTGCAACACA | TTGTCCAGAGTAGTGGGGCA |
| CD19 | Nm_009844 | TGGTGGAGGTAGAAGAGGGA | AGGAAGGGTGTTGACTGGTT |
| CD80 | NM_009855 | AGCTGACTTCTCTACCCCCAA | TCCAACCAAGAGAAGCGAGG |
| CD86 | NM_019388 | TCTGCCGTGCCCATTTACAA | TGTGCCCAAATAGTGCTCGT |
| CD69 | NM_001033122 | GGAGAGAGGGCAGAAGGACCA | TGAGGACCACTATTAACACAGCC |

### 2.7.6.3 Assessment of Local Immune Response: Flow Cytometry

Mice were inoculated with B16-F10 s.c. flank tumors and treated as described above. After 14 or 18 days, or 3 or 7 days after the final treatment, n=4 mice per group were euthanized. Tumors were excised, cut into 2-mm pieces, and digested with collagenase D (Sigma Aldrich) for 1 hr at 37° C. The digested tissue was pressed through a 70-μm cell strainer with a pestle and washed with cold 1×PBS. The cells were pelleted by centrifugation at 300 rcf for 5 min at 4° C. and the supernatant removed. The cells were then resuspended in 1 mL ACK lysing buffer for 1 min at room temperature, then diluted in 10 mL cold 1×PBS, passed through a 100-μm cell strainer, and centrifuged at 300 rcf for 5 min at 4° C.

The supernatant was aspirated, and the cell pellet was resuspended in FACS buffer (1×PBS with 2% FBS) and separated into three aliquots for staining. All samples were centrifuged again to pellet the cells, and the supernatant was removed and replaced with a cocktail of antibodies to stain for (1) CD3ε and CD8a, (2) CD3ε and CD49b, or (3) CD4 and Foxp3. Details of all antibodies used are described in Table 5. The cells were resuspended in the antibody cocktail and incubated on ice and protected from light for 20 min, then washed three times in FACS buffer by centrifugation. Samples stained for intracellular Foxp3 were first stained for CD4 as described here, then fixed, permeabilized, and stained for Foxp3, and washed using the anti-mouse/rat Foxp3 APC staining set (eBioscience, Thermo Fisher, Carlsbad, Calif.) according to the manufacturer's instructions. All samples were finally resuspended in FACS buffer for analysis by flow cytometry using the Accuri C6 with Hypercyt attachment. The gating strategy used is shown in FIG. 27. Differences in cell populations were detected by one-way ANOVA with Dunnett post-tests against the control (i.t. control nanoparticle administration only). Differences were considered statistically significant for p<0.05. Normality of the distributions was confirmed by Shapiro-Wilk tests.

TABLE 5
Antibodies used for flow cytometry

| Protein target | Conjugate | Clone | Dilution | Supplier | Catalog number |
|---|---|---|---|---|---|
| CD3ε | Alexa Fluor 488 (AF488) | 17A2 | 1:50 | BioLegend | 100210 |
| CD8a | Allophycocyanin (APC) | 53-6.7 | 1:100 | BioLegend | 100712 |
| CD49b | Allophycocyanin (APC) | DX5 | 1:100 | BioLegend | 108909 |
| CD4 | Alexa Fluor 488 (AF488) | RM4-5 | 1:80 | BioLegend | 100529 |
| Foxp3 | Allophycocyanin (APC) | FJK-16s | 1:100 | eBioscience | 17-5773-82 |

2.7.7 In Vivo Anti-Tumor Efficacy of tAPC Reprogramming Nanoparticles in MC38 Model

For MC38 anti-tumor efficacy studies, female nine-week-old C57BL/6J mice were inoculated s.c. with 5×10⁵ cells on the right flank as described above. The study was carried out according to the procedure and schedule described for the B16-F10 model. For the MC38 model, only the lead treatment nanoparticle group (4-1BBL/IL-12-encoding DNA nanoparticles i.t.) and a control (fLuc-encoding DNA nanoparticles i.t.) were tested, both with or without anti-PD-1 antibody administered i.p. N=8 mice were assigned to each group. The tumor re-challenge was carried out on long-term surviving mice by inoculating mice on the left flank s.c. with 5×10⁵ cells/mouse and following procedures described above for the B16-F10 model. Differences in tumor size detected by two-way repeated-measures ANOVA with post hoc Tukey tests. Differences in survival curves were detected by Mantel-Cox log-rank tests, with a Bonferroni correction for multiple comparisons.

### 2.7.8 Assessment of Systemic B16-F10 Tumor-Specific Immune Response

Mice were inoculated with B16-F10 s.c. flank tumors and treated as described above, with n=4 per group. At t=12 days post-tumor inoculation, B16-F10 or MC38 cells were seeded in vitro into 96-well plates, then transfected with 4-1BBL and IL-12 in combination on t=13 days as described above. On t=14 days, mice were euthanized by CO₂ asphyxiation, and their spleens were excised and pressed through 70-μm cell strainers using pestles. The red blood cells were lysed using ACK lysing buffer as described above. The CD8+ T cells in each spleen were isolated using MACS negative isolation kits and columns as described above. To each well of B16-F10 or MC38 tAPCs, 10⁵ CD8+ T cells were added in 50 μL complete RPMI growth medium, with a final volume of 150 μL per well, and the co-culture was incubated at 37° C. with 5% CO₂. After 18 hr of incubation, the media from the co-cultures were analyzed by IFN-γ ELISA as described above.

The isolated CD8+ T cells from the spleens of treated and control mice were also stained with a phycoerythrin (PE)-labeled gp100-loaded MHC I tetramer (gp100-Tet; MBL International Corporation, Sunnyvale, Calif.) to quantify the proportion of gp100-specific CD8+ T cells. Following the manufacturer's instructions, 4×105 CD8+ T cells per sample were stained in in 60 μL volume, consisting of FACS buffer with 0.1% sodium azide and 1 μg TruStain FcX anti-CD16/32 antibody (Biolegend) along with 10 μL gp100-Tet. Cells were incubated on ice in the dark for 60 min, then washed twice with FACS buffer and resuspended in 200 μL PBS with 0.5% formaldehyde. Stained cells were incubated on ice in the dark for an additional 1 hr, then analyzed by flow cytometry (Attune NxT).

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
J. M. Redman, G. T. Gibney, M. B. Atkins, Advances in immunotherapy for melanoma. BMC Med. 14, 20 (2016).
I. Mellman, G. Coukos, G. Dranoff, Cancer immunotherapy comes of age. Nature 480, 480-489 (2011).
E. Ben-Akiva, R. A. Meyer, D. R. Wilson, J. J. Green, Surface engineering for lymphocyte programming. Adv. Drug Deliv. Rev. 114, 102-115 (2017).
J. M. Curtsinger et al., Inflammatory cytokines provide a third signal for activation of naive CD4+ and CD8+ T cells. J. Immunol. 162, 3256-3262 (1999).
J. Banchereau, A. K. Palucka, Dendritic cells as therapeutic vaccines against cancer. Nat. Rev. Immunol. 5, 296-306 (2005).
P. W. Kantoff et al.; IMPACT Study Investigators, Sipuleucel-T immunotherapy for castration-resistant prostate cancer. N. Engl. J. Med. 363, 411-422 (2010).
S. Anguille, E. L. Smits, E. Lion, V. F. van Tendeloo, Z. N. Berneman, Clinical use of dendritic cells for cancer therapy. Lancet Oncol. 15, e257-e267 (2014).
P. J. Tacken, I. J. M. de Vries, R. Torensma, C. G. Figdor, Dendritic-cell immunotherapy: From ex vivo loading to in vivo targeting. Nat. Rev. Immunol. 7, 790-802 (2007).
C. Wang, W. Sun, Y. Ye, H. N. Bomba, Z. Gu, Bioengineering of artificial antigen presenting cells and lymphoid organs. Theranostics 7, 3504-3516 (2017).
L. J. Eggermont, L. E. Paulis, J. Tel, C. G. Figdor, Towards efficient cancer immunotherapy: Advances in developing artificial antigen-presenting cells. Trends Biotechnol. 32, 456-465 (2014).
R. F. Wang, H. Y. Wang, Immune targets and neoantigens for cancer immunotherapy and precision medicine. Cell Res. 27, 11-37 (2017).
K. Shi, M. Haynes, L. Huang, Nanovaccines for remodeling the suppressive tumor microenvironment: New horizons in cancer immunotherapy. Front. Chem. Sci. Eng. 11, 676-684 (2017).
D. S. Chen, I. Mellman, Oncology meets immunology: The cancer-immunity cycle. Immunity 39, 1-10 (2013).
J. D. Comber, R. Philip, MHC class I antigen presentation and implications for developing a new generation of therapeutic vaccines. Ther. Adv. Vaccines 2, 77-89 (2014).
H. Zhang et al., 4-1BB is superior to CD28 co-stimulation for generating CD8+ cytotoxic lymphocytes for adoptive immunotherapy. J. Immunol. 179, 4910-4918 (2007).
J. A. Chacon et al., Co-stimulation through 4-1BB/CD137 improves the expansion and function of CD8(+) melanoma tumor-infiltrating lymphocytes for adoptive T-cell therapy. PLoS One 8, e60031 (2013).
M. Xu et al., Regulation of antitumor immune responses by the IL-12 family cytokines, IL-12, IL-23, and IL-27. Clin. Dev. Immunol. 2010, 832454 (2010).
J. Ni, M. Miller, A. Stojanovic, N. Garbi, A. Cerwenka, Sustained effector function of IL-12/15/18-preactivated NK cells against established tumors. J. Exp. Med. 209, 2351-2365 (2012).
T. Bartkowiak, M. A. Curran, 4-1BB agonists: Multi-potent potentiators of tumor immunity. Front. Oncol. 5, 117 (2015).
J. Hsu et al., Contribution of NK cells to immunotherapy mediated by PD-1/PD-L1 blockade. J. Clin. Invest. 128, 4654-4668 (2018).
P. C. Emtage et al., Adenoviral vectors expressing lymphotactin and interleukin 2 or lymphotactin and interleukin 12 synergize to facilitate tumor regression in murine breast cancer models. Hum. Gene Ther. 10, 697-709 (1999).
I. Narvaiza et al., Intra-tumoral co-injection of two adenoviruses, one encoding the chemokine IFN-gamma-inducible protein-10 and another encoding IL-12, results in marked antitumoral synergy. J. Immunol. 164, 3112-3122 (2000).
T. Nomura, H. Hasegawa, M. Kohno, M. Sasaki, S. Fujita, Enhancement of anti-tumor immunity by tumor cells transfected with the secondary lymphoid tissue chemokine EBI-1-ligand chemokine and stromal cell-derived factor-1 alpha chemokine genes. Int. J. Cancer 91, 597-606 (2001).
Y. Fan et al., Immunogenic cell death amplified by co-localized adjuvant delivery for cancer immunotherapy. Nano Lett. 17, 7387-7393 (2017).
M. C. Hanson et al., Nanoparticulate STING agonists are potent lymph node-targeted vaccine adjuvants. J. Clin. Invest. 125,2532-2546 (2015).
N. Cheng et al., A nanoparticle-incorporated STING activator enhances antitumor immunity in PD-L1-insensitive models of triple-negative breast cancer. JCI Insight 3, 120638 (2018).
G. Zhu et al., Intertwining DNA-RNA nanocapsules loaded with tumor neoantigens as synergistic nanovaccines for cancer immunotherapy. Nat. Commun. 8, 1482 (2017).
W. Lasek, R. Zagożdżon, M. Jakobisiak, Interleukin 12: Still a promising candidate for tumor immunotherapy? Cancer Immunol. Immunother. 63,419-435 (2014).
A. M. Di Giacomo, M. Biagioli, M. Maio, The emerging toxicity profiles of anti-CTLA-4 antibodies across clinical indications. Semin. Oncol. 37,499-507 (2010).
J. P. Leonard et al., Effects of single-dose interleukin-12 exposure on interleukin-12-associated toxicity and interferon-gamma production. Blood 90,2541-2548 (1997).
Z. Cheng et al., In vivo expansion and antitumor activity of co-infused CD28- and 4-1BB-engineered CAR-T cells in patients with B cell leukemia. Mol. Ther. 26,976-985 (2018).
C. M. Guenther et al., Synthetic virology: Engineering viruses for gene delivery. Wiley Interdiscip. Rev. Nanomed. Nanobiotechnol. 6,548-558 (2014).
S. Y. Tzeng, L. J. Higgins, M. G. Pomper, J. J. Green, Biomaterial-mediated cancer-specific DNA delivery to liver cell cultures using synthetic poly(beta-amino ester)s. J. Biomed. Mater. Res. A 101A, 1837-1845 (2013).
H. Guerrero-Cázares et al., Biodegradable polymeric nanoparticles show high efficacy and specificity at DNA delivery to human glioblastoma in vitro and in vivo. ACS Nano 8,5141-5153 (2014).
H. Y. Xue, S. Liu, H. L. Wong, Nanotoxicity: A key obstacle to clinical translation of siRNA-based nanomedicine. Nanomedicine (Lond.) 9,295-312 (2014).
D. P. Vangasseri et al., Immunostimulation of dendritic cells by cationic liposomes. Mol. Membr. Biol. 23,385-395 (2006).
A. Mangraviti et al., Polymeric nanoparticles for non-viral gene therapy extend brain tumor survival in vivo. ACS Nano 9, 1236-1249 (2015).
C. M. Diaz-Montero et al., Priming of naive CD8+ T cells in the presence of IL-12 selectively enhances the survival of CD8+CD62 Lhi cells and results in superior anti-tumor activity in a tolerogenic murine model. Cancer Immunol. Immunother. 57, 563-572 (2008).
K. Gee, C. Guzzo, N. F. Che Mat, W. Ma, A. Kumar, The IL-12 family of cytokines in infection, inflammation and autoimmune disorders. Inflamm. Allergy Drug Targets 8, 40-52 (2009).
Y. Nakamura et al., Correlation between vitiligo occurrence and clinical benefit in advanced melanoma patients treated with nivolumab: A multi-institutional retrospective study. J. Dermatol. 44, 117-122 (2017).
L. Chen et al., Co-stimulation of antitumor immunity by the B7 counterreceptor for the T lymphocyte molecules CD28 and CTLA-4. Cell 71, 1093-1102 (1992).
S. E. Townsend, J. P. Allison, Tumor rejection after direct costimulation of CD8+ T cells by B7-transfected melanoma cells. Science 259, 368-370 (1993).
S. Baskar et al., Constitutive expression of B7 restores immunogenicity of tumor cells expressing truncated major histocompatibility complex class II molecules. Proc. Natl. Acad. Sci. U.S.A. 90, 5687-5690 (1993).
K. Hiroishi, T. Tilting, H. Tahara, M. T. Lotze, Interferon-alpha gene therapy in combination with CD80 transduction reduces tumorigenicity and growth of established tumor in poorly immunogenic tumor models. Gene Ther. 6, 1988-1994 (1999).
C. G. Zamboni et al., Polymeric nanoparticles as cancer-specific DNA delivery vectors to human hepatocellular carcinoma. J. Controlled Release 263, 18-28 (2017).
J. Kim, Y. Kang, S. Y. Tzeng, J. J. Green, Synthesis and application of poly(ethylene glycol)-co-poly(β-amino ester) copolymers for small cell lung cancer gene therapy. Acta Biomater. 41, 293-301 (2016).
N. S. Bhise, K. J. Wahlin, D. J. Zack, J. J. Green, Evaluating the potential of poly(beta-amino ester) nanoparticles for reprogramming human fibroblasts to become induced pluripotent stem cells. Int. J. Nanomedicine 8, 4641-4658 (2013).
D. R. Wilson et al., Continuous microfluidic assembly of biodegradable poly(beta-amino ester)/DNA nanoparticles for enhanced gene delivery. J. Biomed. Mater. Res. A 105, 1813-1825 (2017).
S. L. Hewitt et al., Durable anticancer immunity from intra-tumoral administration of IL-23, IL-36γ, and OX40L mRNAs. Sci. Transl. Med. 11, eaat9143 (2019).
R. Nanda et al., Pembrolizumab in Patients with Advanced Triple-Negative Breast Cancer: Phase 1b Keynote-012 Study. Journal of Clinical Oncology, 34, 2460 (2016)
M. J. Smyth, D. I. Godfrey, J. A. Trapani, A Fresh Look at Tumor Immunosurveillance and Immunotherapy, Nature Immunology, 2, 293-299 (2001).

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A composition comprising at least one of a first genetic element that encodes a signal 2 protein and a second genetic element that encodes a signal 3 protein encapsulated in a nanoparticle comprising a cationic biomaterial or biomaterial blend.

2. The composition of claim 1, further comprising a third genetic element that encodes a signal 1 protein.

3. The composition of claim 1, wherein the signal 2 protein is a cell surface bound protein that regulates immune cells.

4. The composition of claim 3, wherein the signal 2 protein is selected from the group consisting of 4-1BBL, CD80, CD86, and OX40L.

5. The composition of claim 1, wherein the signal 3 protein is a secreted protein that regulates immune cells.

6. The composition of claim 5, wherein the signal 3 protein comprises a cytokine.

7. The composition of claim 6, wherein the cytokine comprises an interleukin.

8. The composition of claim 5, wherein the signal 3 protein is selected from the group consisting of IL-2, IL-12, IL-6, IL-7, IL-15, IL-18, IL-21, IFN-α, and IFN-β.

9. The composition of claim 2, wherein the signal 1 protein is major histocompatibility complex (MHC) I/human leukocyte antigen (HLA) I or MHC II/HLA II.

10. The composition of claim 1, wherein the cationic biomaterial comprises one or more cationic polymers.

11. The composition of claim 10, wherein the one or more cationic polymers comprises one or more cationic biodegradable polymers.

12. The composition of claim 11, wherein the one or more cationic degradable polymers comprises one or more poly(beta-amino ester)s (PBAEs).

13. The composition of claim 12, wherein the one or more PBAEs comprises a compound of formula (I): 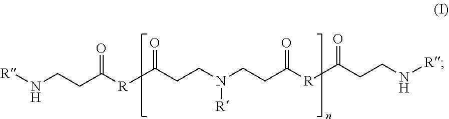
wherein:
n is an integer from 1 to 10,000;
each R is independently selected from the group consisting of: 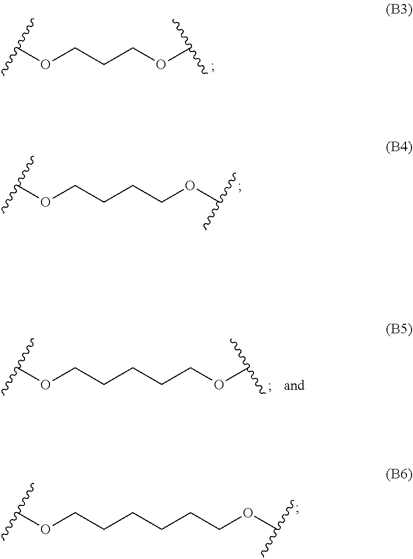 [SMILES: *OCCCO* ; *OCCCCO* ; *OCCCCCO* ; *OCCCCCCO*]
each R′ is independently selected from the group consisting of: 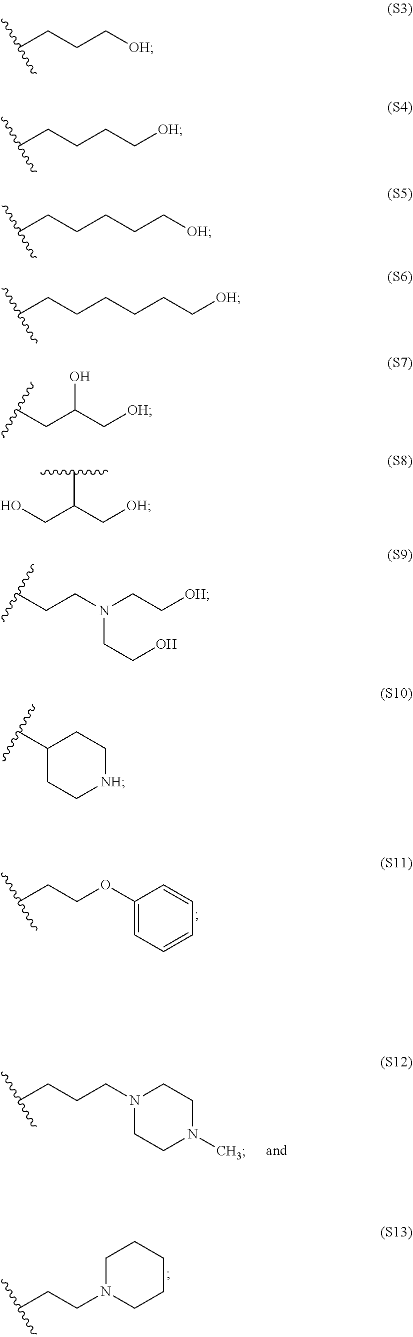
each R″ is independently selected from the group consisting of: 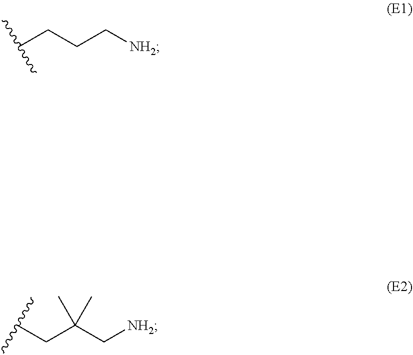 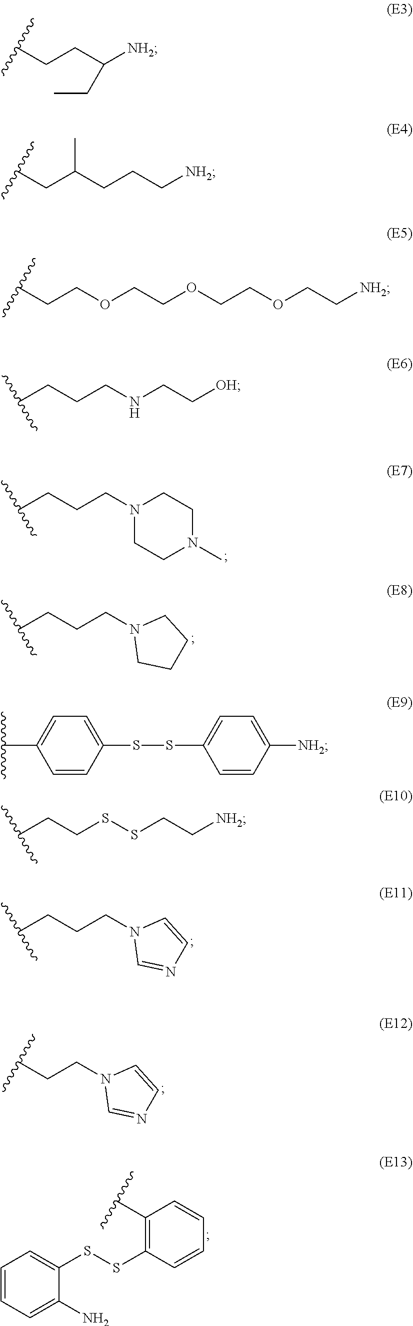 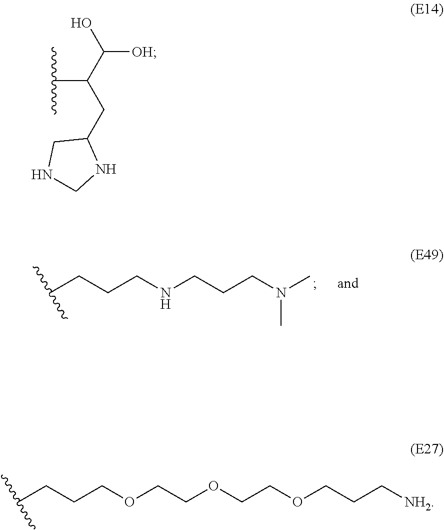 [SMILES: *C(CC1CNCN1)C(O)O ; *CCCNCCCN(C)C ; *CCCOCCOCCOCCCN]
and pharmaceutically acceptable salts thereof.

14. The composition of claim 13, wherein the one or more PBAEs is selected from the group consisting of:

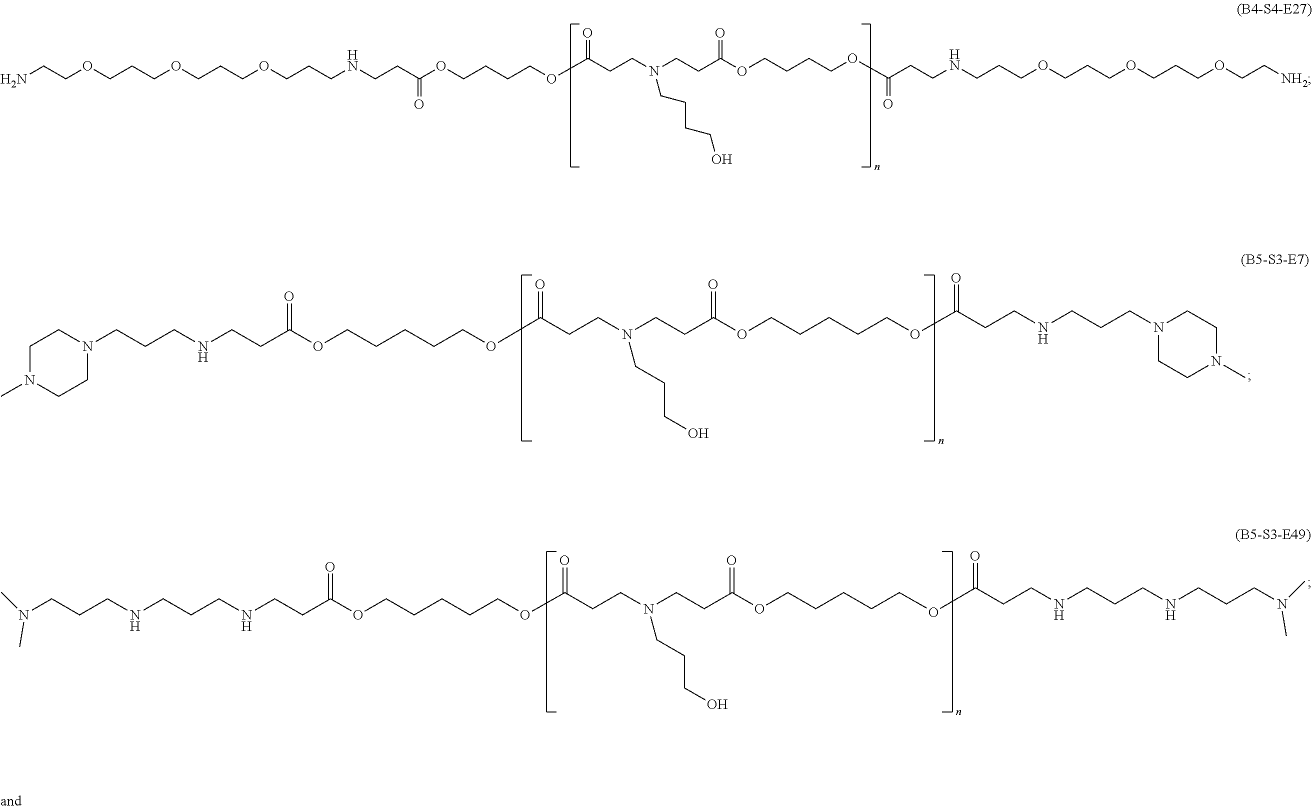

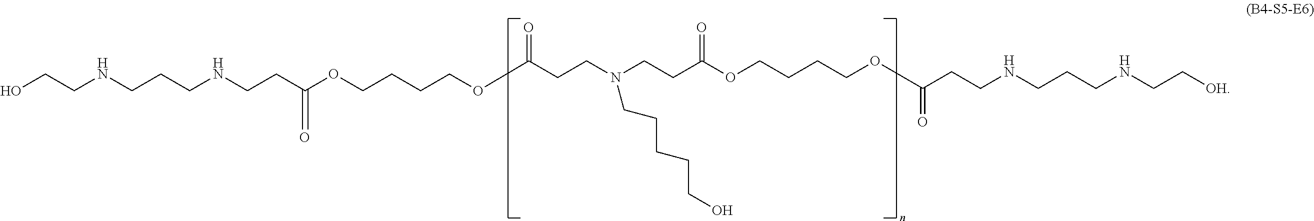

15. The composition of claim 13, wherein n is selected from the group consisting of an integer from 1 to 1,000; an integer from 1 to 100; an integer from 1 to 30; an integer from 5 to 20; an integer from 10 to 15; and an integer from 1 to 10.

16. The composition of claim 1, wherein the nanoparticle has a size ranging from about 20 nm to about 50 nm; from about 50 nm to about 200 nm; or from about 200 to about 500 nm.

17. A method for reprogramming one or more cancer cells into one or more tumor-derived antigen-presenting cells (tAPCs), wherein the one or more tAPCs mimic a natural antigen-presenting cell (APC) and direct an immune response against themselves and other cancer cells, the method comprising transfecting the one or more cancer cells with composition of any of claims 1-16.

18. The method of claim 17, wherein the transfection of the one or more cancer cells promotes an immune cell activation against one or more antigens expressed on the one or more cancer cells.

19. The method of claim 17, wherein the one or more tAPCs activate an antigen-specific T-cell response against MHC I+ tumor cells.

20. The method of claim 17, wherein the one or more tAPCs provide an activating signal to one or more natural killer (NK) cells to induce anti-tumor cytotoxicity therein.

21. The method of claim 17, wherein the one or more tAPCs activate an antigen-independent NK cell response against MHC I−/low tumor cells.

22. The method of claim 17, further inducing a systemic immune response resulting in cell death of distant metastases.

23. A method of treating cancer, the method comprising administering to a subject in need of treatment thereof a composition of any of claims 1-16.

24. The method of claim 23, wherein the cancer is selected from the group consisting of a melanoma, a breast cancer, colorectal cancer, liver cancer, and brain cancer.

25. A pharmaceutical formulation of the composition of any of claims 1-16 in a pharmaceutically acceptable carrier.

26. A kit comprising the composition of any of claims 1-16.

27. The composition of claim 1, used in combination with one or more anti-cancer immune checkpoint inhibitor molecules, such as anti-PD-1 antibody.


SEQUENCE LISTING (44 sequences)

SEQ ID NO: 1 - DNA, 20 bp, Artificial sequence
  Synthetic
ggctgtattcccctccatcg

SEQ ID NO: 2 - DNA, 20 bp, Artificial sequence
  Synthetic
atcactctgggcttgctgat

SEQ ID NO: 3 - DNA, 20 bp, Artificial sequence
  Synthetic
aagaggaggtggagttgtgg

SEQ ID NO: 4 - DNA, 20 bp, Artificial sequence
  Synthetic
agcccaccttcgttgtctat

SEQ ID NO: 5 - DNA, 20 bp, Artificial sequence
  Synthetic
acaccaccgatttctggcta

SEQ ID NO: 6 - DNA, 20 bp, Artificial sequence
  Synthetic
acctatgccacccttatccg

SEQ ID NO: 7 - DNA, 20 bp, Artificial sequence
  Synthetic
agccctgtcagacatcaaca

SEQ ID NO: 8 - DNA, 20 bp, Artificial sequence
  Synthetic
gaatgctgtgtttgcctgga

SEQ ID NO: 9 - DNA, 20 bp, Artificial sequence
  Synthetic
catggctgtttctggctgtt

SEQ ID NO: 10 - DNA, 20 bp, Artificial sequence
  Synthetic
acggaatacagggctttcga

SEQ ID NO: 11 - DNA, 20 bp, Artificial sequence
  Synthetic
cccgggatgagacagttgat

SEQ ID NO: 12 - DNA, 20 bp, Artificial sequence
  Synthetic
ccacagtttctcccaccaca

SEQ ID NO: 13 - DNA, 20 bp, Artificial sequence
  Synthetic
ggttgtagagccgggtgaaa

SEQ ID NO: 14 - DNA, 21 bp, Artificial sequence
  Synthetic
acctgtaaacgaggcttcctg

SEQ ID NO: 15 - DNA, 20 bp, Artificial sequence
  Synthetic
agtgctgggagacgtgaatg

SEQ ID NO: 16 - DNA, 22 bp, Artificial sequence
  Synthetic
acgtggagaactttgatgcttt

SEQ ID NO: 17 - DNA, 21 bp, Artificial sequence
  Synthetic
cttcttgtggccctactgtgt

SEQ ID NO: 18 - DNA, 21 bp, Artificial sequence
  Synthetic
cctgagacttcctgcaacaca

SEQ ID NO: 19 - DNA, 20 bp, Artificial sequence
  Synthetic
tggtggaggtagaagaggga

SEQ ID NO: 20 - DNA, 21 bp, Artificial sequence
  Synthetic
agctgacttctctacccccaa

SEQ ID NO: 21 - DNA, 20 bp, Artificial sequence
  Synthetic
tctgccgtgcccatttacaa

SEQ ID NO: 22 - DNA, 21 bp, Artificial sequence
  Synthetic
ggagagagggcagaaggacca

SEQ ID NO: 23 - DNA, 22 bp, Artificial sequence
  Synthetic
ccagttggtaacaatgccatgt

SEQ ID NO: 24 - DNA, 20 bp, Artificial sequence
  Synthetic
tgggctcatagtctgggttg

SEQ ID NO: 25 - DNA, 19 bp, Artificial sequence
  Synthetic
gtttgcactctgtcagggg

SEQ ID NO: 26 - DNA, 20 bp, Artificial sequence
  Synthetic
agccttcgttttccttgctg

SEQ ID NO: 27 - DNA, 20 bp, Artificial sequence
  Synthetic
agtctgtggtggttatgggg

SEQ ID NO: 28 - DNA, 20 bp, Artificial sequence
  Synthetic
gcgaacatgcgagtaaacca

SEQ ID NO: 29 - DNA, 20 bp, Artificial sequence
  Synthetic
tgggagtacttggtgcgaat

SEQ ID NO: 30 - DNA, 20 bp, Artificial sequence
  Synthetic
tctggattggggctgaagaa

SEQ ID NO: 31 - DNA, 20 bp, Artificial sequence
  Synthetic
tccttttgccagttcctcca

SEQ ID NO: 32 - DNA, 20 bp, Artificial sequence
  Synthetic
ccggttcatgtcatggatgg

SEQ ID NO: 33 - DNA, 20 bp, Artificial sequence
  Synthetic
attctgcgcacttgttcctg

SEQ ID NO: 34 - DNA, 20 bp, Artificial sequence
  Synthetic
gtgtagttcccaagagcccc

SEQ ID NO: 35 - DNA, 20 bp, Artificial sequence
  Synthetic
tcttgtggtcagggtgcttc

SEQ ID NO: 36 - DNA, 23 bp, Artificial sequence
  Synthetic
ctgagttaggaccacaaggtgag

SEQ ID NO: 37 - DNA, 20 bp, Artificial sequence
  Synthetic
gcactgaggctggctattga

SEQ ID NO: 38 - DNA, 21 bp, Artificial sequence
  Synthetic
tactgctgcttggtgtctctg

SEQ ID NO: 39 - DNA, 20 bp, Artificial sequence
  Synthetic
ttggcactccatagcactcg

SEQ ID NO: 40 - DNA, 20 bp, Artificial sequence
  Synthetic
ttgtccagagtagtggggca

SEQ ID NO: 41 - DNA, 20 bp, Artificial sequence
  Synthetic
aggaagggtgttgactggtt

SEQ ID NO: 42 - DNA, 20 bp, Artificial sequence
  Synthetic
tccaaccaagagaagcgagg

SEQ ID NO: 43 - DNA, 20 bp, Artificial sequence
  Synthetic
tgtgcccaaatagtgctcgt

SEQ ID NO: 44 - DNA, 23 bp, Artificial sequence
  Synthetic
tgaggaccactattaacacagcc